SEQUENCE LISTING (400 sequences)

SEQ ID NO: 1 - protein, 120 aa, synthetic construct
EVQLVESGGGLVKPGGSLRLSCAASGFTFSGYGLSWVRQAPGKGLEWVSTISSGGGYTHYAPSVKGRFTISRDNAKNSLYLQMNSLRAEDTAVYYCARHPRDFSYAQDYWGQGTTVTVSS

SEQ ID NO: 2 - protein, 107 aa, synthetic construct
EIVMTQSPATLSVSPGERATLSCKASQDVGHYVAWYQQKPGQAPRLLIYWASTRATGIPARFSGSGSGTEFTLTISSLQSEDFAVYYCQQYNIYPWTFGQGTKVEIK

SEQ ID NO: 3 - protein, 120 aa, synthetic construct
EVQLVESGGGLVKPGGSLRLSCAASGFTFSHYGVSWVRQAPGKGLEWVSTISSGGGYTHYAQSVKGRFTISRDNAKNSLYLQMNSLRAEDTAVYYCARHPRDFSYAQDYWGQGTTVTVSS

SEQ ID NO: 4 - protein, 107 aa, synthetic construct
EIVMTQSPATLSVSPGERATLSCKASQDVGHYVAWYQQKPGQAPRLLIYWASTRHTGIPARFSGSGSGTEFTLTISSLQSEDFAVYYCQQYNIYPWTFGQGTKVEIK

SEQ ID NO: 5 - protein, 120 aa, synthetic construct
EVQLVESGGGLVKPGGSLRLSCAASGFTFSSYGHSWVRQAPGKGLEWVSTISSGGGYTHYAPSVKGRFTISRDNAKNSLYLQMNSLRAEDTAVYYCARHPRDFSYAHDYWGQGTTVTVSS

SEQ ID NO: 6 - protein, 107 aa, synthetic construct
EIVMTQSPATLSVSPGERATLSCRASQDVGHYVAWYQQKPGQAPRLLIYWASTRATGIPARFSGSGSGTEFTLTISSLQSEDFAVYYCQQYNIYPWTFGQGTKVEIK

SEQ ID NO: 7 - protein, 120 aa, synthetic construct
EVQLVESGGGLVKPGGSLRLSCAASGFTFSQYGHSWVRQAPGKGLEWVSTISSGGGYTHYAESVKGRFTISRDNAKNSLYLQMNSLRAEDTAVYYCARHPRDFSYAQDYWGQGTTVTVSS

SEQ ID NO: 8 - protein, 107 aa, synthetic construct
EIVMTQSPATLSVSPGERATLSCKASQDVGHYLAWYQQKPGQAPRLLIYWASTRATGIPARFSGSGSGTEFTLTISSLQSEDFAVYYCQQYNIYPWTFGQGTKVEIK

SEQ ID NO: 9 - protein, 120 aa, synthetic construct
EVQLVESGGGLVKPGGSLRLSCAASGFTFSKYGTSWVRQAPGKGLEWVSTISSGGGYTHYAPSVKGRFTISRDNAKNSLYLQMNSLRAEDTAVYYCARHPRDFSYAQDYWGQGTTVTVSS

SEQ ID NO: 10 - protein, 107 aa, synthetic construct; identical to SEQ ID NO: 6

SEQ ID NO: 11 - protein, 120 aa, synthetic construct
EVQLVESGGGLVKPGGSLRLSCAASGFTFSHYGVSWVRQAPGKGLEWVSTISSGGGYTHYVASVKGRFTISRDNAKNSLYLQMNSLRAEDTAVYYCARHPRDFSYALDYWGQGTTVTVSS

SEQ ID NO: 12 - protein, 107 aa, synthetic construct
EIVMTQSPATLSVSPGERATLSCKASQDVGHYVAWYQQKPGQAPRLLIYWASTRATGIPARFSGSGSGTEFTLTISSLQSEDFAVYYCQQYNIYPWAFGQGTKVEIK

SEQ ID NO: 13 - protein, 120 aa, synthetic construct
EVQLVESGGGLVKPGGSLRLSCAASGFTFSVYGLSWVRQAPGKGLEWVSTISSGGGYTQYAPSVKGRFTISRDNAKNSLYLQMNSLRAEDTAVYYCARHPRDFSYAFDYWGQGTTVTVSS

SEQ ID NO: 14 - protein, 107 aa, synthetic construct; identical to SEQ ID NO: 8

SEQ ID NO: 15 - protein, 120 aa, synthetic construct
EVQLVESGGGLVKPGGSLRLSCAASGFTFSAYGTSWVRQAPGKGLEWVSTISSGGGYTYYAASVKGRFTISRDNAKNSLYLQMNSLRAEDTAVYYCARHPRDFSYAIDYWGQGTTVTVSS

SEQ ID NO: 16 - protein, 107 aa, synthetic construct
EIVMTQSPATLSVSPGERATLSCRASQDVGHYLAWYQQKPGQAPRLLIYWASTRATGIPARFSGSGSGTEFTLTISSLQSEDFAVYYCQQYNIYPWTFGQGTKVEIK

SEQ ID NO: 17 - protein, 120 aa, synthetic construct
EVQLVESGGGLVKPGGSLRLSCAASGFTFSAYGSSWVRQAPGKGLEWVSTISSGGGYTHYAPSVKGRFTISRDNAKNSLYLQMNSLRAEDTAVYYCARHPRDFSYAHDYWGQGTTVTVSS

SEQ ID NO: 18 - protein, 107 aa, synthetic construct; identical to SEQ ID NO: 4

SEQ ID NO: 19 - protein, 120 aa, synthetic construct
EVQLVESGGGLVKPGGSLRLSCAASGFTFSSYGISWVRQAPGKGLEWVSTISSGGGYTHYAHSVKGRFTISRDNAKNSLYLQMNSLRAEDTAVYYCARHPRDFSYALDYWGQGTTVTVSS

SEQ ID NO: 20 - protein, 107 aa, synthetic construct; identical to SEQ ID NO: 8

SEQ ID NO: 21 - protein, 120 aa, synthetic construct
EVQLVESGGGLVKPGGSLRLSCAASGFTFSQYGHSWVRQAPGKGLEWVSTISSGGGYTHYAHSVKGRFTISRDNAKNSLYLQMNSLRAEDTAVYYCARHPRDFSYANDYWGQGTTVTVSS

SEQ ID NO: 22 - protein, 107 aa, synthetic construct; identical to SEQ ID NO: 16

SEQ ID NO: 23 - protein, 120 aa, synthetic construct
EVQLVESGGGLVKPGGSLRLSCAASGFTFSQYGHSWVRQAPGKGLEWVSTISSGGGYTHYADSVKGRFTISRDNAKNSLYLQMNSLRAEDTAVYYCARHPRDFSYAQDYWGQGTTVTVSS

SEQ ID NO: 24 - protein, 107 aa, synthetic construct
EIVMTQSPATLSVSPGERATLSCKASQDVGHYLAWYQQKPGQAPRLLIYWASTRHTGIPARFSGSGSGTEFTLTISSLQSEDFAVYYCQQYNIYPWTFGQGTKVEIK

SEQ ID NO: 25 - protein, 120 aa, synthetic construct
EVQLVESGGGLVKPGGSLRLSCAASGFTFSHYGTSWVRQAPGKGLEWVSTISSGGGYTHYAPSVKGRFTISRDNAKNSLYLQMNSLRAEDTAVYYCARHPRDFSYAIDYWGQGTTVTVSS

SEQ ID NO: 26 - protein, 107 aa, synthetic construct; identical to SEQ ID NO: 2

SEQ ID NO: 27 - protein, 120 aa, synthetic construct
EVQLVESGGGLVKPGGSLRLSCAASGFTFSKYGLSWVRQAPGKGLEWVSTISSGGGYTHYVESVKGRFTISRDNAKNSLYLQMNSLRAEDTAVYYCARHPRDFSYASDYWGQGTTVTVSS

SEQ ID NO: 28 - protein, 107 aa, synthetic construct; identical to SEQ ID NO: 2

SEQ ID NO: 29 - protein, 120 aa, synthetic construct
EVQLVESGGGLVKPGGSLRLSCAASGFTFSTYGASWVRQAPGKGLEWVSTISSGGGYTHYAPSVKGRFTISRDNAKNSLYLQMNSLRAEDTAVYYCARHPRDFSYALDYWGQGTTVTVSS

SEQ ID NO: 30 - protein, 107 aa, synthetic construct; identical to SEQ ID NO: 2

SEQ ID NO: 31 - protein, 120 aa, synthetic construct
EVQLVESGGGLVKPGGSLRLSCAASGFTFSTYGVSWVRQAPGKGLEWVSTISSGGGYTHYAGSVKGRFTISRDNAKNSLYLQMNSLRAEDTAVYYCARHPRDFSYALDYWGQGTTVTVSS

SEQ ID NO: 32 - protein, 107 aa, synthetic construct; identical to SEQ ID NO: 16

SEQ ID NO: 33 - DNA, 2832 bp, synthetic construct
  source 1..2832: mol_type = other DNA
atggcccggggctcggcgctcccgcggcggccgctgctgtgcatcccggccgtctgggcggccgccgcgcttctgctctcagtgtcccggacttcaggtgaagtggaggttctggatccgaacgaccctttaggaccccttgatgggcaggacggcccgattccaactctgaaaggttactttctgaattttctggagccagtaaacaatatcaccattgtccaaggccagacggcaattctgcactgcaaggtggcaggaaacccaccccctaacgtgcggtggctaaagaatgatgccccggtggtgcaggagccgcggcggatcatcatccggaagacagaatatggttcacgactgcgaatccaggacctggacacgacagacactggctactaccagtgcgtggccaccaacgggatgaagaccattaccgccactggcgtcctgtttgtgcggctgggtccaacgcacagcccaaatcataactttcaggatgattaccacgaggatgggttctgccagccttaccggggaattgcctgtgcacgcttcattggcaaccggaccatttatgtggactcgcttcagatgcagggggagattgaaaaccgaatcacagcggccttcaccatgatcggcacgtctacgcacctgtcggaccagtgctcacagttcgccatcccatccttctgccacttcgtgtttcctctgtgcgacgcgcgctcccgggcacccaagccgcgtgagctgtgccgcgacgagtgcgaggtgctggagagcgacctgtgccgccaggagtacaccatcgcccgctccaacccgctcatcctcatgcggcttcagctgcccaagtgtgaggcgctgcccatgcctgagagccccgacgctgccaactgcatgcgcattggcatcccagccgagaggctgggccgctaccatcagtgctataacggctcaggcatggattacagaggaacggcaagcaccaccaagtcaggccaccagtgccagccgtgggccctgcagcacccccacagccaccacctgtccagcacagacttccctgagcttggaggggggcacgcctactgccggaaccccggaggccagatggagggcccctggtgctttacgcagaataaaaacgtacgcatggaactgtgtgacgtaccctcgtgtagtccccgagacagcagcaagatggggattctgtacatcttggtccccagcatcgcaattccactggtcatcgcttgccttttcttcttggtttgcatgtgccggaataagcagaaggcatctgcgtccacaccgcagcggcgacagctgatggcctcgcccagccaagacatggaaatgcccctcattaaccagcacaaacaggccaaactcaaagagatcagcctgtctgcggtgaggttcatggaggagctgggagaggaccggtttgggaaagtctacaaaggtcacctgttcggccctgccccgggggagcagacccaggctgtggccatcaaaacgctgaaggacaaagcggaggggcccctgcgggaggagttccggcatgaggctatgctgcgagcacggctgcaacaccccaacgtcgtctgcctgctgggcgtggtgaccaaggaccagcccctgagcatgatcttcagctactgttcgcacggcgacctccacgaattcctggtcatgcgctcgccgcactcggacgtgggcagcaccgatgatgaccgcacggtgaagtccgccctggagccccccgacttcgtgcaccttgtggcacagatcgcggcggggatggagtacctatccagccaccacgtggttcacaaggacctggccacccgcaatgtgctagtgtacgacaagctgaacgtgaagatctcagacttgggcctcttccgagaggtgtatgccgccgattactacaagctgctggggaactcgctgctgcctatccgctggatggccccagaggccatcatgtacggcaagttctccatcgactcagacatctggtcctacggtgtggtcctgtgggaggtcttcagctacggcctgcagccctactgcgggtactccaaccaggatgtggtggagatgatccggaaccggcaggtgctgccttgccccgatgactgtcccgcctgggtgtatgccctcatgatcgagtgctggaacgagttccccagccggcggccccgcttcaaggacatccacagccggctccgagcctggggcaacctttccaactacaacagctcggcgcagacctcgggggccagcaacaccacgcagaccagctccctgagcaccagcccagtgagcaatgtgagcaacgcccgctacgtggggcccaagcagaaggccccgcccttcccacagccccagttcatccccatgaagggccagatcagacccatggtgcccccgccgcagctctacgtccccgtcaacggctaccagccggtgccggcctatggggcctacctgcccaacttctacccggtgcagatcccaatgcagatggccccgcagcaggtgcctcctcagatggtccccaagcccagctcacaccacagtggcagtggctccaccagcacaggctacgtcaccacggccccctccaacacatccatggcagacagggcagccctgctctcagagggcgctgatgacacacagaacgccccagaagatggggcccagagcaccgtgcaggaagcagaggaggaggaggaaggctctgtcccagagactgagctgctgggggactgtgacactctgcaggtggacgaggcccaagtccagctggaagcttga

SEQ ID NO: 34 - protein, 943 aa, synthetic construct
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNVVCLLGVVTKDQPLSMIFSYCSHGDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYVGPKQKAPPFPQPQFIPMKGQIRPMVPPPQLYVPVNGYQPVPAYGAYLPNFYPVQIPMQMAPQQVPPQMVPKPSSHHSGSGSTSTGYVTTAPSNTSMADRAALLSEGADDTQNAPEDGAQSTVQEAEEEEEGSVPETELLGDCDTLQVDEAQVQLEA

SEQ ID NO: 35 - DNA, 2835 bp, synthetic construct
  source 1..2835: mol_type = other DNA
atggctcggggctgggtgcggccgagccgtgtgcctctgtgcgcccgggccgtctggacggctgcggcgctcctgctctggacaccctggacggcaggtgaagtggaagattcggaggcaatcgacaccttgggacaacctgatggaccggacagcccacttcccactctgaaaggctactttctgaattttctggagccagtcaacaatatcaccattgttcagggccagacggcaatcctgcactgcaaggtggcgggaaacccacctcccaatgtgcggtggctgaagaatgatgccccggttgtgcaagagccacgaagggtcgtcatccggaagacagaatacggctcccggctgcggatccaagacctggacacaacagacacaggctactaccagtgtgtggctaccaacgggctgaagaccatcactgccactggggttctatatgtgcggctcggtccgacgcacagcccgaaccacaattttcaggatgacgatcaggaagatggcttctgccagccgtaccgagggatcgcttgtgcgcgcttcattgggaaccggactatttatgtggactccctccagatgcagggggagattgaaaaccgaatcacagctgccttcaccatgatcggcacctccacgcaactgtcagaccagtgttcacagtttgccatcccatccttctgccacttcgtcttccctctgtgcgacgcatgctcccgggcgcccaagcctcgcgaactgtgccgggatgaatgtgaggtgctggagaacgacctgtgccgccaggagtacaccatcgcccgctccaacccgctcatcctcatgcggctccagctgcccaagtgcgaagcgctgcccatgcccgagagcccggatgctgcgaactgcatgcgcatcgggatccccgcggagaggctgggtcgctaccaccagtgctacaacggctccggcgccgattacagggggatggccagtaccaccaagtcaggccaccagtgtcagccttgggctctgcagcacccccacagccatcgcctatccagcacggaattccctgagctgggaggaggccatgcctactgccggaaccccgggggccagatggaaggcccgtggtgctttacgcagaataaaaacgtacgcgtggaactgtgtgacgtacccccgtgtagtccccgatatggcagcaagatggggattctgtacatcctggtccccagcattgctatccccctggtcatcgcttgcctgttcttcctcgtctgcatgtgccgcaacaaacagaaggcttcggcctccaccccacagcgccggcagctgatggcctctcccagccaggacatggagatgccactcatcagccagcacaaacaggccaaactcaaagagatcagcttgtccacagtgaggttcatggaggagctcggggaggaccggtttggcaaggtctacaaaggccacctgttcgggcctgccccaggagaaccaacccaggccgtggccatcaagacgctgaaagacaaggctgaggggcccctgcgggaggagttccggcaagaggcgatgctccgggcccgactgcagcaccccaacatcgtctgtctcctaggcgtcgtgaccaaggaccaacccttgagcatgatcttcagctactgttcccatggcgaccttcatgaattcctggtcatgcgctcgccgcactccgatgtgggcagcaccgatgacgaccgcacagtgaagtcagccctggagcccccggacttcgtgcacgtggtggcgcagatcgctgcggggatggagttcctgtccagccaccacgtgtgccataaggacctggccacacgcaatgtgctggtgtacgacaagctgaacgtgaggatctcagacttgggcctcttccgtgaggtatactccgcagattactacaaactcatgggcaattcactgctgcccatccgctggatgtcccccgaggccgtcatgtatggaaagttctccatcgactctgacatctggtcctacggtgtggtcctctgggaggtctttagctacggcctgcagccctactgtgggtactccaaccaggacgtggtggagatgatccggagccggcaggtgctgccctgcccggatgactgccccgcctgggtctatgccctcatgattgaatgctggaatgagttcccaagccggaggccccgctttaaggacatccacagccggctccggtcctggggcaacctatccaactataatagttccgcgcagacctcaggagccagcaacaccacacagaccagctccctgagcaccagccccgtaagcaatgtgagcaatgcccgctatatggcccccaagcagaaggcccagcccttcccacagcctcagttcatccccatgaagggtcagatcagacccttggtgccccccgcacagctgtacatcccggtgaacggctatcagccggtaccggcatacggggcctacctgcccaacttctacccagtccagatccccatgcagatggccccacagcaggtgccccctcagatggtccccaagccgagctcacaccacagtggcagcggctccaccagcactggctacgtcaccacggcgccctccaatacatctgtggcggacagggcggccctactctctgagggcaccgaggatgtacagaacatcgcggaagacgtggcccagagccctgtgcaggaagcagaggaggaggaggaggggtctgtccctgagactgaactcctgggagacaatgacacgctccaggtgaccgaggcggctcatgtccagcttgaagcctga

SEQ ID NO: 36 - protein, 944 aa, synthetic construct
MARGWVRPSRVPLCARAVWTAAALLLWTPWTAGEVEDSEAIDTLGQPDGPDSPLPTLKGYFLNFLEPVNNITIVQGQTAILHCKVAGNPPPNVRWLKNDAPVVQEPRRVVIRKTEYGSRLRIQDLDTTDTGYYQCVATNGLKTITATGVLYVRLGPTHSPNHNFQDDDQEDGFCQPYRGIACARFIGNRTIYVDSLQMQGEIENRITAAFTMIGTSTQLSDQCSQFAIPSFCHFVFPLCDACSRAPKPRELCRDECEVLENDLCRQEYTIARSNPLILMRLQLPKCEALPMPESPDAANCMRIGIPAERLGRYHQCYNGSGADYRGMASTTKSGHQCQPWALQHPHSHRLSSTEFPELGGGHAYCRNPGGQMEGPWCFTQNKNVRVELCDVPPCSPRYGSKMGILYILVPSIAIPLVIACLFFLVCMCRNKQKASASTPQRRQLMASPSQDMEMPLISQHKQAKLKEISLSTVRFMEELGEDRFGKVYKGHLFGPAPGEPTQAVAIKTLKDKAEGPLREEFRQEAMLRARLQHPNIVCLLGVVTKDQPLSMIFSYCSHGDLHEFLVMRSPHSDVGSTDDDRTVKSALEPPDFVHVVAQIAAGMEFLSSHHVCHKDLATRNVLVYDKLNVRISDLGLFREVYSADYYKLMGNSLLPIRWMSPEAVMYGKFSIDSDIWSYGVVLWEVFSYGLQPYCGYSNQDVVEMIRSRQVLPCPDDCPAWVYALMIECWNEFPSRRPRFKDIHSRLRSWGNLSNYNSSAQTSGASNTTQTSSLSTSPVSNVSNARYMAPKQKAQPFPQPQFIPMKGQIRPLVPPAQLYIPVNGYQPVPAYGAYLPNFYPVQIPMQMAPQQVPPQMVPKPSSHHSGSGSTSTGYVTTAPSNTSVADRAALLSEGTEDVQNIAEDVAQSPVQEAEEEEEGSVPETELLGDNDTLQVTEAAHVQLEA

SEQ ID NO: 37 - DNA, 1209 bp, synthetic construct
  source 1..1209: mol_type = other DNA
atggcccggggctcggcgctcccgcggcggccgctgctgtgcatcccggccgtctgggcggccgccgcgcttctgctctcagtgtcccggacttcaggtgaagtggaggttctggatccgaacgaccctttaggaccccttgatgggcaggacggcccgattccaactctgaaaggttactttctgaattttctggagccagtaaacaatatcaccattgtccaaggccagacggcaattctgcactgcaaggtggcaggaaacccaccccctaacgtgcggtggctaaagaatgatgccccggtggtgcaggagccgcggcggatcatcatccggaagacagaatatggttcacgactgcgaatccaggacctggacacgacagacactggctactaccagtgcgtggccaccaacgggatgaagaccattaccgccactggcgtcctgtttgtgcggctgggtccaacgcacagcccaaatcataactttcaggatgattaccacgaggatgggttctgccagccttaccggggaattgcctgtgcacgcttcattggcaaccggaccatttatgtggactcgcttcagatgcagggggagattgaaaaccgaatcacagcggccttcaccatgatcggcacgtctacgcacctgtcggaccagtgctcacagttcgccatcccatccttctgccacttcgtgtttcctctgtgcgacgcgcgctcccgggcacccaagccgcgtgagctgtgccgcgacgagtgcgaggtgctggagagcgacctgtgccgccaggagtacaccatcgcccgctccaacccgctcatcctcatgcggcttcagctgcccaagtgtgaggcgctgcccatgcctgagagccccgacgctgccaactgcatgcgcattggcatcccagccgagaggctgggccgctaccatcagtgctataacggctcaggcatggattacagaggaacggcaagcaccaccaagtcaggccaccagtgccagccgtgggccctgcagcacccccacagccaccacctgtccagcacagacttccctgagcttggaggggggcacgcctactgccggaaccccggaggccagatggagggcccctggtgctttacgcagaataaaaacgtacgcatggaactgtgtgacgtaccctcgtgtagtccccgagacagcagcaagatgggg

SEQ ID NO: 38 - protein, 403 aa, synthetic construct
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMG

SEQ ID NO: 39 - DNA, 1209 bp, synthetic construct
  source 1..1209: mol_type = other DNA
atggctcggggctgggtgcggccgagccgtgtgcctctgtgcgcccgggccgtctggacggctgcggcgctcctgctctggacaccctggacggcaggtgaagtggaagattcggaggcaatcgacaccttgggacaacctgatggaccggacagcccacttcccactctgaaaggctactttctgaattttctggagccagtcaacaatatcaccattgttcagggccagacggcaatcctgcactgcaaggtggcgggaaacccacctcccaatgtgcggtggctgaagaatgatgccccggttgtgcaagagccacgaagggtcgtcatccggaagacagaatacggctcccggctgcggatccaagacctggacacaacagacacaggctactaccagtgtgtggctaccaacgggctgaagaccatcactgccactggggttctatatgtgcggctcggtccgacgcacagcccgaaccacaattttcaggatgacgatcaggaagatggcttctgccagccgtaccgagggatcgcttgtgcgcgcttcattgggaaccggactatttatgtggactccctccagatgcagggggagattgaaaaccgaatcacagctgccttcaccatgatcggcacctccacgcaactgtcagaccagtgttcacagtttgccatcccatccttctgccacttcgtcttccctctgtgcgacgcatgctcccgggcgcccaagcctcgcgaactgtgccgggatgaatgtgaggtgctggagaacgacctgtgccgccaggagtacaccatcgcccgctccaacccgctcatcctcatgcggctccagctgcccaagtgcgaagcgctgcccatgcccgagagcccggatgctgcgaactgcatgcgcatcgggatccccgcggagaggctgggtcgctaccaccagtgctacaacggctccggcgccgattacagggggatggccagtaccaccaagtcaggccaccagtgtcagccttgggctctgcagcacccccacagccatcgcctatccagcacggaattccctgagctgggaggaggccatgcctactgccggaaccccgggggccagatggaaggcccgtggtgctttacgcagaataaaaacgtacgcgtggaactgtgtgacgtacccccgtgtagtccccgatatggcagcaagatgggg

SEQ ID NO: 40 - protein, 403 aa, synthetic construct
MARGWVRPSRVPLCARAVWTAAALLLWTPWTAGEVEDSEAIDTLGQPDGPDSPLPTLKGYFLNFLEPVNNITIVQGQTAILHCKVAGNPPPNVRWLKNDAPVVQEPRRVVIRKTEYGSRLRIQDLDTTDTGYYQCVATNGLKTITATGVLYVRLGPTHSPNHNFQDDDQEDGFCQPYRGIACARFIGNRTIYVDSLQMQGEIENRITAAFTMIGTSTQLSDQCSQFAIPSFCHFVFPLCDACSRAPKPRELCRDECEVLENDLCRQEYTIARSNPLILMRLQLPKCEALPMPESPDAANCMRIGIPAERLGRYHQCYNGSGADYRGMASTTKSGHQCQPWALQHPHSHRLSSTEFPELGGGHAYCRNPGGQMEGPWCFTQNKNVRVELCDVPPCSPRYGSKMG

SEQ ID NO: 41 - protein, 82 aa, synthetic construct
HQCYNGSGMDYRGTASTTKSGHQCQPWALQHPHSHHLSSTDFPELGGGHAYCRNPGGQMEGPWCFTQNKNVRMELCDVPSCS

SEQ ID NO: 42 - protein, 69 aa, synthetic construct
AILHCKVAGNPPPNVRWLKNDAPVVQEPRRIIIRKTEYGSRLRIQDLDTTDTGYYQCVATNGMKTITAT

SEQ ID NO: 43 - protein, 8 aa, synthetic construct
GFTFSNYG

SEQ ID NO: 44 - protein, 10 aa, synthetic construct
GFTFSNYGMS

SEQ ID NO: 45 - protein, 8 aa, synthetic construct
ISSGGGYT

SEQ ID NO: 46 - protein, 12 aa, synthetic construct
TISSGGGYTHYV

SEQ ID NO: 47 - protein, 13 aa, synthetic construct
ARHPRDFSYAMDY

SEQ ID NO: 48 - protein, 13 aa, synthetic construct
ARHPRDFSYAMDY

SEQ ID NO: 49 - protein, 6 aa, synthetic construct
QDVGHY

SEQ ID NO: 50 - protein, 11 aa, synthetic construct
KASQDVGHYVA

SEQ ID NO: 51 - skipped

SEQ ID NO: 52 - protein, 7 aa, synthetic construct
WASTRHT

SEQ ID NO: 53 - protein, 9 aa, synthetic construct
QQYNIYPWT

SEQ ID NO: 54 - protein, 9 aa, synthetic construct
QQYNIYPWT

SEQ ID NO: 55 - protein, 120 aa, synthetic construct
EVQLVESGGGLVKPGGSLRLSCAASGFTFSNYGMSWVRQAPGKGLEWVSTISSGGGYTHYVDSVKGRFTISRDNAKNSLYLQMNSLRAEDTAVYYCARHPRDFSYAMDYWGQGTTVTVSS

SEQ ID NO: 56 - protein, 120 aa, synthetic construct
QVQLLESGGGLVKPGGSLRLSCAASGFTFSNYGMSWIRQAPGKGLEWVSTISSGGGYTHYVDSVKGRFTISRDNAKNSLYLQMNSLRAEDTAVYYCARHPRDFSYAMDYWGQGTTVTVSS

SEQ ID NO: 57 - protein, 120 aa, synthetic construct
EVQLVESGGGLVKPGGSLRLSCAASGFTFSNYGMNWVRQAPGKGLEWVSSISSGGGYTYYADSVKGRFTISRDNAKNSLYLQMNSLRAEDTAVYYCARHPRDFSYAMDYWGQGTTVTVSS

SEQ ID NO: 58 - protein, 120 aa, synthetic construct
QVQLLESGGGLVKPGGSLRLSCAASGFTFSNYGMSWIRQAPGKGLEWVSYISSGGGYTNYADSVKGRFTISRDNAKNSLYLQMNSLRAEDTAVYYCARHPRDFSYAMDYWGQGTTVTVSS

SEQ ID NO: 59 - protein, 120 aa, synthetic construct; identical to SEQ ID NO: 55

SEQ ID NO: 60 - protein, 107 aa, synthetic construct
AIRMTQSPSSFSASTGDRVTITCRASQDVGHYLAWYQQKPGKAPKLLIYWASTLQSGVPSRFSGSGSGTDFTLTISCLQSEDFATYYCQQYNIYPWTFGQGTKVEIK

SEQ ID NO: 61 - protein, 120 aa, synthetic construct; identical to SEQ ID NO: 55

SEQ ID NO: 62 - protein, 107 aa, synthetic construct; identical to SEQ ID NO: 16

SEQ ID NO: 63 - protein, 107 aa, synthetic construct
DIQMTQSPSSVSASVGDRVTITCKASQDVGHYVAWYQQKPGKAPKLLIYWASTRHTGVPSRFSGSGSGTDFTLTISSLQPEDFATYYCQQYNIYPWTFGQGTKVEIK

SEQ ID NO: 64 - protein, 107 aa, synthetic construct
DIQMTQSPSSVSASVGDRVTITCRASQDVGHYLAWYQQKPGKAPKLLIYWASSLQSGVPSRFSGSGSGTDFTLTISSLQPEDFATYYCQQYNIYPWTFGQGTKVEIK

SEQ ID NO: 65 - protein, 120 aa, synthetic construct
EVQLVESGGGLVKPGGSLRLSCAASGFTFSKYGISWVRQAPGKGLEWVSTISSGGGYTHYADSVKGRFTISRDNAKNSLYLQMNSLRAEDTAVYYCARHPRDFSYALDYWGQGTTVTVSS

SEQ ID NO: 66 - protein, 107 aa, synthetic construct; identical to SEQ ID NO: 2

SEQ ID NO: 67 - protein, 120 aa, synthetic construct
EVQLVESGGGLVKPGGSLRLSCAASGFTFSSYGISWVRQAPGKGLEWVSTISSGGGYTHYAESVKGRFTISRDNAKNSLYLQMNSLRAEDTAVYYCARHPRDFSYAMDYWGQGTTVTVSS

SEQ ID NO: 68 - protein, 107 aa, synthetic construct
EIVMTQSPATLSVSPGERATLSCRASQDVGHYLAWYQQKPGQAPRLLIYWASTRHTGIPARFSGSGSGTEFTLTISSLQSEDFAVYYCQQYNIYPWTFGQGTKVEIK

SEQ ID NO: 69 - protein, 120 aa, synthetic construct
EVQLVESGGGLVKPGGSLRLSCAASGFTFSQYGTSWVRQAPGKGLEWVSTISSGGGYTHYAESVKGRFTISRDNAKNSLYLQMNSLRAEDTAVYYCARHPRDFSYAHDYWGQGTTVTVSS

SEQ ID NO: 70 - protein, 107 aa, synthetic construct; identical to SEQ ID NO: 68

SEQ ID NO: 71 - protein, 120 aa, synthetic construct
EVQLVESGGGLVKPGGSLRLSCAASGFTFSGYGMSWVRQAPGKGLEWVSTISSGGGYTHYAASVKGRFTISRDNAKNSLYLQMNSLRAEDTAVYYCARHPRDFSYAFDYWGQGTTVTVSS

SEQ ID NO: 72 - protein, 107 aa, synthetic construct; identical to SEQ ID NO: 8

SEQ ID NO: 73 - protein, 120 aa, synthetic construct
EVQLVESGGGLVKPGGSLRLSCAASGFTFSGYGLSWVRQAPGKGLEWVSTISSGGGYTHYAASVKGRFTISRDNAKNSLYLQMNSLRAEDTAVYYCARHPRDFSYAMDYWGQGTTVTVSS

SEQ ID NO: 74 - protein, 107 aa, synthetic construct; identical to SEQ ID NO: 2

SEQ ID NO: 75 - protein, 120 aa, synthetic construct
EVQLVESGGGLVKPGGSLRLSCAASGFTFSTYGMSWVRQAPGKGLEWVSTISSGGGYTHYATSVKGRFTISRDNAKNSLYLQMNSLRAEDTAVYYCARHPRDFSYAHDYWGQGTTVTVSS

SEQ ID NO: 76 - protein, 107 aa, synthetic construct; identical to SEQ ID NO: 16

SEQ ID NO: 77 - protein, 120 aa, synthetic construct; identical to SEQ ID NO: 1

SEQ ID NO: 78 - protein, 107 aa, synthetic construct; identical to SEQ ID NO: 2

SEQ ID NO: 79 - protein, 120 aa, synthetic construct
EVQLVESGGGLVKPGGSLRLSCAASGFTFSQYGTSWVRQAPGKGLEWVSSISSGGGYTHYAESVKGRFTISRDNAKNSLYLQMNSLRAEDTAVYYCARHPRDFSYAFDYWGQGTTVTVSS

SEQ ID NO: 80 - protein, 107 aa, synthetic construct; identical to SEQ ID NO: 2

SEQ ID NO: 81 - protein, 120 aa, synthetic construct
EVQLVESGGGLVKPGGSLRLSCAASGFTFSGYGMNWVRQAPGKGLEWVSTISSGGGYTHYAESVKGRFTISRDNAKNSLYLQMNSLRAEDTAVYYCARHPRDFSYAADYWGQGTTVTVSS

SEQ ID NO: 82 - protein, 107 aa, synthetic construct; identical to SEQ ID NO: 8

SEQ ID NO: 83 - protein, 120 aa, synthetic construct
EVQLVESGGGLVKPGGSLRLSCAASGFTFSNYGMSWVRQAPGKGLEWVSTISSGGGYTHYVASVKGRFTISRDNAKNSLYLQMNSLRAEDTAVYYCARHPRDFSYAMDYWGQGTTVTVSS

SEQ ID NO: 84 - protein, 107 aa, synthetic construct; identical to SEQ ID NO: 8

SEQ ID NO: 85 - protein, 120 aa, synthetic construct
EVQLVESGGGLVKPGGSLRLSCAASGFTFSGYGMSWVRQAPGKGLEWVSTISSGGGYTHYATSVKGRFTISRDNAKNSLYLQMNSLRAEDTAVYYCARHPRDFSYAMDYWGQGTTVTVSS

SEQ ID NO: 86 - protein, 107 aa, synthetic construct; identical to SEQ ID NO: 8

SEQ ID NO: 87 - protein, 120 aa, synthetic construct
EVQLVESGGGLVKPGGSLRLSCAASGFTFSAYGASWVRQAPGKGLEWVSSISSGGGYTHYVESVKGRFTISRDNAKNSLYLQMNSLRAEDTAVYYCARHPRDFSYAQDYWGQGTTVTVSS

SEQ ID NO: 88 - protein, 107 aa, synthetic construct; identical to SEQ ID NO: 16

SEQ ID NO: 89 - protein, 120 aa, synthetic construct
EVQLVESGGGLVKPGGSLRLSCAASGFTFSKYGISWVRQAPGKGLEWVSTISSGGGYTHYAASVKGRFTISRDNAKNSLYLQMNSLRAEDTAVYYCARHPRDFSYAQDYWGQGTTVTVSS

SEQ ID NO: 90 - protein, 107 aa, synthetic construct; identical to SEQ ID NO: 8

SEQ ID NO: 91 - protein, 120 aa, synthetic construct
EVQLVESGGGLVKPGGSLRLSCAASGFTFSSYGMNWVRQAPGKGLEWVSTISSGGGYTHYVDSVKGRFTISRDNAKNSLYLQMNSLRAEDTAVYYCARHPRDFSYATDYWGQGTTVTVSS

SEQ ID NO: 92 - protein, 107 aa, synthetic construct
EIVMTQSPATLSVSPGERATLSCRASQDVGHYVAWYQQKPGQAPRLLIYWASTRHTGIPARFSGSGSGTEFTLTISSLQSEDFAVYYCQQYNIYPWTFGQGTKVEIK

SEQ ID NO: 93 - protein, 120 aa, synthetic construct
EVQLVESGGGLVKPGGSLRLSCAASGFTFSNYGLSWVRQAPGKGLEWVSTISSGGGYTHYVTSVKGRFTISRDNAKNSLYLQMNSLRAEDTAVYYCARHPRDFSYALDYWGQGTTVTVSS

SEQ ID NO: 94 - protein, 107 aa, synthetic construct; identical to SEQ ID NO: 4

SEQ ID NO: 95 - protein, 120 aa, synthetic construct
EVQLVESGGGLVKPGGSLRLSCAASGFTFSAYGMSWVRQAPGKGLEWVSTISSGGGYTHYATSVKGRFTISRDNAKNSLYLQMNSLRAEDTAVYYCARHPRDFSYAQDYWGQGTTVTVSS

SEQ ID NO: 96 - protein, 107 aa, synthetic construct; identical to SEQ ID NO: 4

SEQ ID NO: 97 - protein, 120 aa, synthetic construct
EVQLVESGGGLVKPGGSLRLSCAASGFTFSRYGMSWVRQAPGKGLEWVSSISSGGGYTHYAESVKGRFTISRDNAKNSLYLQMNSLRAEDTAVYYCARHPRDFSYAMDYWGQGTTVTVSS

SEQ ID NO: 98 - protein, 107 aa, synthetic construct; identical to SEQ ID NO: 24

SEQ ID NO: 99 - protein, 120 aa, synthetic construct
EVQLVESGGGLVKPGGSLRLSCAASGFTFSGYGVSWVRQAPGKGLEWVSTISSGGGYTHYADSVKGRFTISRDNAKNSLYLQMNSLRAEDTAVYYCARHPRDFSYALDYWGQGTTVTVSS

SEQ ID NO: 100 - protein, 107 aa, synthetic construct; identical to SEQ ID NO: 16

SEQ ID NO: 101 - protein, 120 aa, synthetic construct
EVQLVESGGGLVKPGGSLRLSCAASGFTFSRYGVSWVRQAPGKGLEWVSTISSGGGYTHYAASVKGRFTISRDNAKNSLYLQMNSLRAEDTAVYYCARHPRDFSYAQDYWGQGTTVTVSS

SEQ ID NO: 102 - protein, 107 aa, synthetic construct; identical to SEQ ID NO: 2

SEQ ID NO: 103 - protein, 120 aa, synthetic construct
EVQLVESGGGLVKPGGSLRLSCAASGFTFSKYGVSWVRQAPGKGLEWVSTISSGGGYTHYAPSVKGRFTISRDNAKNSLYLQMNSLRAEDTAVYYCARHPRDFSYAMDYWGQGTTVTVSS

SEQ ID NO: 104 - protein, 107 aa, synthetic construct; identical to SEQ ID NO: 8

SEQ ID NO: 105 - protein, 120 aa, synthetic construct
EVQLVESGGGLVKPGGSLRLSCAASGFTFSSYGMSWVRQAPGKGLEWVSTISSGGGYTHYVASVKGRFTISRDNAKNSLYLQMNSLRAEDTAVYYCARHPRDFSYALDYWGQGTTVTVSS

SEQ ID NO: 106 - protein, 107 aa, synthetic construct; identical to SEQ ID NO: 8

SEQ ID NO: 107 - protein, 120 aa, synthetic construct
EVQLVESGGGLVKPGGSLRLSCAASGFTFSKYGTSWVRQAPGKGLEWVSTISSGGGYTHYAPSVKGRFTISRDNAKNSLYLQMNSLRAEDTAVYYCARHPRDFSYAMDYWGQGTTVTVSS

SEQ ID NO: 108 - protein, 107 aa, synthetic construct; identical to SEQ ID NO: 2

SEQ ID NO: 109 - protein, 120 aa, synthetic construct
EVQLVESGGGLVKPGGSLRLSCAASGFTFSSYGMSWVRQAPGKGLEWVSTISSGGGYTHYAPSVKGRFTISRDNAKNSLYLQMNSLRAEDTAVYYCARHPRDFSYALDYWGQGTTVTVSS

SEQ ID NO: 110 - protein, 107 aa, synthetic construct; identical to SEQ ID NO: 8

SEQ ID NO: 111 - protein, 120 aa, synthetic construct
EVQLVESGGGLVKPGGSLRLSCAASGFTFSEYGMSWVRQAPGKGLEWVSTISSGGGYTHYVESVKGRFTISRDNAKNSLYLQMNSLRAEDTAVYYCARHPRDFSYAMDYWGQGTTVTVSS

SEQ ID NO: 112 - protein, 107 aa, synthetic construct; identical to SEQ ID NO: 6

SEQ ID NO: 113 - protein, 120 aa, synthetic construct
EVQLVESGGGLVKPGGSLRLSCAASGFTFSHYGHSWVRQAPGKGLEWVSTISSGGGYTHYVESVKGRFTISRDNAKNSLYLQMNSLRAEDTAVYYCARHPRDFSYAMDYWGQGTTVTVSS

SEQ ID NO: 114 - protein, 107 aa, synthetic construct; identical to SEQ ID NO: 6

SEQ ID NO: 115 - protein, 120 aa, synthetic construct
EVQLVESGGGLVKPGGSLRLSCAASGFTFSRYGMSWVRQAPGKGLEWVSTISSGGGYTHYAASVKGRFTISRDNAKNSLYLQMNSLRAEDTAVYYCARHPRDFSYAMDYWGQGTTVTVSS

SEQ ID NO: 116 - protein, 107 aa, synthetic construct; identical to SEQ ID NO: 4

SEQ ID NO: 117 - protein, 120 aa, synthetic construct
EVQLVESGGGLVKPGGSLRLSCAASGFTFSTYGLSWVRQAPGKGLEWVSTISSGGGYTHYVDSVKGRFTISRDNAKNSLYLQMNSLRAEDTAVYYCARHPRDFSYAMDYWGQGTTVTVSS

SEQ ID NO: 118 - protein, 107 aa, synthetic construct; identical to SEQ ID NO: 2

SEQ ID NO: 119 - protein, 120 aa, synthetic construct
EVQLVESGGGLVKPGGSLRLSCAASGFTFSPYGMSWVRQAPGKGLEWVSTISSGGGYTHYVQSVKGRFTISRDNAKNSLYLQMNSLRAEDTAVYYCARHPRDFSYALDYWGQGTTVTVSS

SEQ ID NO: 120 - protein, 107 aa, synthetic construct; identical to SEQ ID NO: 2

SEQ ID NO: 121 - protein, 120 aa, synthetic construct; identical to SEQ ID NO: 3

SEQ ID NO: 122 - protein, 107 aa, synthetic construct; identical to SEQ ID NO: 4

SEQ ID NO: 123 - protein, 120 aa, synthetic construct
EVQLVESGGGLVKPGGSLRLSCAASGFTFSSYGMSWVRQAPGKGLEWVSTISSGGGYTHYAASVKGRFTISRDNAKNSLYLQMNSLRAEDTAVYYCARHPRDFSYAMDYWGQGTTVTVSS

SEQ ID NO: 124 - protein, 107 aa, synthetic construct; identical to SEQ ID NO: 8

SEQ ID NO: 125 - protein, 120 aa, synthetic construct
EVQLVESGGGLVKPGGSLRLSCAASGFTFSSYGMSWVRQAPGKGLEWVSTISSGGGYTHYAESVKGRFTISRDNDKNSLYLQMNSLRAEDTAVYYCARHPRDFSYAIDYWGQGTTVTVSS

SEQ ID NO: 126 - protein, 107 aa, synthetic construct; identical to SEQ ID NO: 92

SEQ ID NO: 127 - protein, 120 aa, synthetic construct
EVQLVESGGGLVKPGGSLRLSCAASGFTFSNYGTSWVRQAPGKGLEWVSTISSGGGYTHYAESVKGRFTISRDNAKNSLYLQMNSLRAEDTAVYYCARHPRDFSYAMDYWGQGTTVTVSS

SEQ ID NO: 128 - protein, 107 aa, synthetic construct; identical to SEQ ID NO: 8

SEQ ID NO: 129 - protein, 120 aa, synthetic construct
EVQLVESGGGLVKPGGSLRLSCAASGFTFSTYGMSWVRQAPGKGLEWVSTISSGGGYTHYVTSVKGRFTISRDNAKNSLYLQMNSLRAEDTAVYYCARHPRDFSYAMDYWGQGTTVTVSS

SEQ ID NO: 130 - protein, 107 aa, synthetic construct; identical to SEQ ID NO: 4

SEQ ID NO: 131 - protein, 120 aa, synthetic construct
EVQLVESGGGLVKPGGSLRLSCAASGFTFSSYGMSWVRQAPGKGLEWVSTISSGGGYTHYVHSVKGRFTISRDNAKNSLYLQMNSLRAEDTAVYYCARHPRDFSYAMDYWGQGTTVTVSS

SEQ ID NO: 132 - protein, 107 aa, synthetic construct
EIVMTPSPATLSVSPGERATLSCKASQDVGHYVAWYQQKPGQAPRLLIYWASTRATGIPARFSGSGSGTEFTLTISSLQSEDFAVYYCQQYNIYPWTFGQGTKVEIK

SEQ ID NO: 133 - protein, 120 aa, synthetic construct
EVQLVESGGGLVKPGGSLRLSCAASGFTFSSYGISWVRQAPGKGLEWVSTISSGGGYTHYADSVKGRFTISRDNAKNSLYLQMNSLRAEDTAVYYCARHPRDFSYAHDYWGQGTTVTVSS

SEQ ID NO: 134 - protein, 107 aa, synthetic construct; identical to SEQ ID NO: 4

SEQ ID NO: 135 - protein, 120 aa, synthetic construct
EVQLVESGGGLVKPGGSLRLSCAASGFTFSAYGHSWVRQAPGKGLEWVSTISSGGGYTHYADSVKGRFTISRDNAKNSLYLQMNSLRAEDTAVYYCARHPRDFSYAQDYWGQGTTVTVSS

SEQ ID NO: 136 - protein, 107 aa, synthetic construct; identical to SEQ ID NO: 24

SEQ ID NO: 137 - protein, 120 aa, synthetic construct; identical to SEQ ID NO: 95

SEQ ID NO: 138 - protein, 107 aa, synthetic construct; identical to SEQ ID NO: 8

SEQ ID NO: 139 - protein, 120 aa, synthetic construct
EVQLVESGGGLVKPGGSLRLSCAASGFTFSRYGMSWVRQAPGKGLEWVSTISSGGGYTHYAPSVKGRFTISRDNAKNSLYLQMNSPRAEDTAVYYCARHPRDFSYAQDYWGQGTTVTVSS

SEQ ID NO: 140 - protein, 107 aa, synthetic construct; identical to SEQ ID NO: 24

SEQ ID NO: 141 - protein, 120 aa, synthetic construct
EVQLVESGGGLVKPGGSLRLSCAASGFTFSNYGQSWVRQAPGKGLEWVSTISSGGGYTHYADSVKGRFTISRDNAKNSLYLQMNSLRAEDTAVYYCARHPRDFSYAVDYWGQGTTVTVSS

SEQ ID NO: 142 - protein, 107 aa, synthetic construct; identical to SEQ ID NO: 4

SEQ ID NO: 143 - protein, 120 aa, synthetic construct; identical to SEQ ID NO: 5

SEQ ID NO: 144 - protein, 107 aa, synthetic construct; identical to SEQ ID NO: 6

SEQ ID NO: 145 - protein, 120 aa, synthetic construct
EVQLVESGGGLVKPGGSLRLSCAASGFTFSGYGVSWVRQAPGKGLEWVSTISSGGGYTHYAPSVKGRFTISRDNAKNSLYLQMNSLRAEDTAVYYCARHPRDFSYAQDYWGQGTTVTVSS

SEQ ID NO: 146 - protein, 107 aa, synthetic construct; identical to SEQ ID NO: 2

SEQ ID NO: 147 - protein, 120 aa, synthetic construct
EVQLVESGGGLVKPGGSLRLSCAASGFTFSGYGLSWVRQAPGKGLEWVSTISSGGGYTHYVESVKGRFTISRDNAKNSLYLQMNSLRAEDTAVYYCARHPRDFSYASDYWGQGTTVTVSS

SEQ ID NO: 148 - protein, 107 aa, synthetic construct; identical to SEQ ID NO: 6

SEQ ID NO: 149 - protein, 120 aa, synthetic construct
EVQLVESGGGLVKPGGSLRLSCAASGFTFSSYGTSWVRQAPGKGLEWVSTISSGGGYTHYATSVKGRFTISRDNAKNSLYLQMNSLRAEDTAVYYCARHPRDFSYAMDYWGQGTTVTVSS

SEQ ID NO: 150 - protein, 107 aa, synthetic construct; identical to SEQ ID NO: 8

SEQ ID NO: 151 - protein, 120 aa, synthetic construct
EVQLVESGGGLVKPGGSLRLSCAASGFTFSTYGSSWVRQAPGKGLEWVSTISSGGGYTHYAQSVKGRFTISRDNAKNSLYLQMNSLRAEDTAVYYCARHPRDFSYALDYWGQGTTVTVSS

SEQ ID NO: 152 - protein, 107 aa, synthetic construct; identical to SEQ ID NO: 4

SEQ ID NO: 153 - protein, 120 aa, synthetic construct
EVQLVESGGGLVKPGGSLRLSCAASGFTFSGYGMSWVRQAPGKGLEWVSTISSGGGYTHYVQSVKGRFTISRDNAKNSLYLQMNSLRAEDTAVYYCARHPRDFSYAMDYWGQGTTVTVSS

SEQ ID NO: 154 - protein, 107 aa, synthetic construct; identical to SEQ ID NO: 2

SEQ ID NO: 155 - protein, 120 aa, synthetic construct
EVQLVESGGGLVKPGGSLRLSCAASGFTFSRYGISWVRQAPGKGLEWVSTISSGGGYTHYATSVKGRFTISRDNAKNSLYLQMNSLRAEDTAVYYCARHPRDFSYAMDYWGQGTTVTVSS

SEQ ID NO: 156 - protein, 107 aa, synthetic construct; identical to SEQ ID NO: 4

SEQ ID NO: 157 - protein, 120 aa, synthetic construct
EVQLVESGGGLVKPGGSLRLSCAASGFTFSKYGMSWVRQAPGKGPEWVSTISSGGGYTHYATSVKGRFTISRDNAKNSLYLQMNSLRAEDTAVYYCARHPRDFSYALDYWGQGTTVTVSS

SEQ ID NO: 158 - protein, 107 aa, synthetic construct; identical to SEQ ID NO: 6

SEQ ID NO: 159 - protein, 120 aa, synthetic construct
EVQLVESGGGLVKPGGSLRLSCAASGFTFSNYGVSWVRQAPGKGLEWVSTISSGGGYTHYVQSVKGRFTISRDNAKNSLYLQMNSLRAEDTAVYYCARHPRDFSYALDYWGQGTTVTVSS

SEQ ID NO: 160 - protein, 107 aa, synthetic construct; identical to SEQ ID NO: 4

SEQ ID NO: 161 - protein, 120 aa, synthetic construct
EVQLVESGGGLVKPGGSLRLSCAASGFTFSNYGMSWVRQAPGKGLEWVSSISSGGGYTHYAESVKGRFTISRDNAKNSLYLQMNSLRAEDTAVYYCARHPRDFSYAMDYWGQGTTVTVSS

SEQ ID NO: 162 - protein, 107 aa, synthetic construct; identical to SEQ ID NO: 2

SEQ ID NO: 163 - protein, 120 aa, synthetic construct
EVQLVESGGGLVKPGGSLRLSCAASGFTFSRYGMSWVRQAPGKGLEWVSTISSGGGYTHYVDSVKGRFTISRDNAKNSLYLQMNSLRAEDTAVYYCARHPRDFSYAHDYWGQGTTVTVSS

SEQ ID NO: 164 - protein, 107 aa, synthetic construct; identical to SEQ ID NO: 8

SEQ ID NO: 165 - protein, 120 aa, synthetic construct
EVQLVESGGGLVKPGGSLRLSCAASGFTFSNYGMSWVRQAPGKGLEWVSTISSGGGYTHYVDSVKGRFTISRDNAKNSLYLQMNSLRAEDTAVYYCARHPRDFSYALDYWGQGTTVTVSS

SEQ ID NO: 166 - protein, 107 aa, synthetic construct; identical to SEQ ID NO: 2

SEQ ID NO: 167 - protein, 120 aa, synthetic construct
EVQLVESGGGLVKPGGSLRLSCAASGFTFSHYGTSWVRQAPGKGLEWVSTISSGGGYTHYAESVKGRFTISRDNAKNSLYLQMNSLRAEDTAVYYCARHPRDFSYAIDYWGQGTTVTVSS

SEQ ID NO: 168 - protein, 107 aa, synthetic construct; identical to SEQ ID NO: 8

SEQ ID NO: 169 - protein, 120 aa, synthetic construct
EVQLVESGGGLVKPGGSLRLSCAASGFTFSKYGVSWVRQAPGKGLEWVSTISSGGGYTHYVDSVKGRFTISRDNAKNSLYLQMNSLRAEDTAVYYCARHPRDFSYAHDYWGQGTTVTVSS

SEQ ID NO: 170 - protein, 107 aa, synthetic construct
EIVMTQSPATLSVSPGERATLSCKASQDVGHYVAWYQQKPGQAPRLLIYWASTRHTGIPARFSGSGSGTEFTLTISSLKSEDFAVYYCQQYNIYPWTFGQGTKVEIK

SEQ ID NO: 171 - protein, 120 aa, synthetic construct
EVQLVESGGGLVKPGGSLRLSCAASGFTFSNYGVSWVRQAPGKGLEWVSTISSGGGYTHYVTSVKGRFTISRDNAKNSLYLQMNSLRAEDTAVYYCARHPRDFSYALDYWGQGTTVTVSS

SEQ ID NO: 172 - protein, 107 aa, synthetic construct; identical to SEQ ID NO: 2

SEQ ID NO: 173 - protein, 120 aa, synthetic construct; identical to SEQ ID NO: 7

SEQ ID NO: 174 - protein, 107 aa, synthetic construct; identical to SEQ ID NO: 8

SEQ ID NO: 175 - protein, 120 aa, synthetic construct; identical to SEQ ID NO: 9

SEQ ID NO: 176 - protein, 107 aa, synthetic construct; identical to SEQ ID NO: 6

SEQ ID NO: 177 - protein, 120 aa, synthetic construct; identical to SEQ ID NO: 11

SEQ ID NO: 178 - protein, 107 aa, synthetic construct; identical to SEQ ID NO: 12

SEQ ID NO: 179 - protein, 120 aa, synthetic construct; identical to SEQ ID NO: 13

SEQ ID NO: 180 - protein, 107 aa, synthetic construct; identical to SEQ ID NO: 8

SEQ ID NO: 181 - protein, 120 aa, synthetic construct
EVQLVESGGGLVKPGGSLRLSCAASGFTFSGYGLSWVRQAPGKGLEWVSTISSGGGYTHYAPSVKGRFTISRDNAKNSLYLQMNSLRAEDTAVYYCARHPRDFSYAFDYWGQGTTVTVSS

SEQ ID NO: 182 - protein, 107 aa, synthetic construct; identical to SEQ ID NO: 8

SEQ ID NO: 183 - protein, 120 aa, synthetic construct
EVQLVESGGGLVKPGGSLRLSCAASGFTFSRYGLSWVRQAPGKGLEWVSTISSGGGYTHYAASVKGRFTISRDNAKNSLYLQMNSLRAEDTAVYYCARHPRDFSYAMDYWGQGTTVTVSS

SEQ ID NO: 184 - protein, 107 aa, synthetic construct; identical to SEQ ID NO: 16

SEQ ID NO: 185 - protein, 120 aa, synthetic construct
EVQLVESGGGLVKPGGSLRLSCAASGFTFSNYGQSWVRQAPGKGLEWVSTISSGGGYTHYAPSVKGRFTISRDNAKNSLYLQMNSLRAEDTAVYYCARHPRDFSYAFDYWGQGTTVTVSS

SEQ ID NO: 186 - protein, 107 aa, synthetic construct; identical to SEQ ID NO: 6

SEQ ID NO: 187 - protein, 120 aa, synthetic construct
EVQLVESGGGLVKPGGSLRLSCAASGFTFSSYGHSWVRQAPGKGLEWVSTISSGGGYTYYAESVKGRFTISRDNAKNSLYLQMNSLRAEDTAVYYCARHPRDFSYAHDYWGQGTTVTVSS

SEQ ID NO: 188 - protein, 107 aa, synthetic construct; identical to SEQ ID NO: 8

SEQ ID NO: 189 - protein, 120 aa, synthetic construct; identical to SEQ ID NO: 15

SEQ ID NO: 190 - protein, 107 aa, synthetic construct; identical to SEQ ID NO: 16

SEQ ID NO: 191 - protein, 120 aa, synthetic construct
EVQLVESGGGLVKPGGSLRLSCAASGFTFSHYGISWVRQAPGKGLEWVSSISSGGGYTHYAESVKGRFTISRDNAKNSLYLQMNSLRAEDTAVYYCARHPRDFSYAMDYWGQGTTVTVSS

SEQ ID NO: 192 - protein, 107 aa, synthetic construct; identical to SEQ ID NO: 6

SEQ ID NO: 193 - protein, 120 aa, synthetic construct
EVQLVESGGGLVKPGGSLRLSCAASGFTFSGYGMSWVRQAPGKGLEWVSTISSGGGYTHYVDSVKGRFTISRDNAKNSLYLQMNSLRAEDTAVYYCARHPRDFSYALDYWGQGTTVTVSS

SEQ ID NO: 194 - protein, 107 aa, synthetic construct; identical to SEQ ID NO: 8

SEQ ID NO: 195 - protein, 120 aa, synthetic construct; identical to SEQ ID NO: 17

SEQ ID NO: 196 - protein, 107 aa, synthetic construct; identical to SEQ ID NO: 4

SEQ ID NO: 197 - protein, 120 aa, synthetic construct
EVQLVESGGGLVKPGGSLRLSCAASGFTFSHYGASWVRQAPGKGLEWVSTISSGGGYTHYAGSVKGRFTISRDNAKNSLYLQMNSLRAEDTAVYYCARHPRDFSYAFDYWGQGTTVTVSS

SEQ ID NO: 198 - protein, 107 aa, synthetic construct; identical to SEQ ID NO: 8

SEQ ID NO: 199 - protein, 120 aa, synthetic construct; identical to SEQ ID NO: 19

SEQ ID NO: 200 - protein, 107 aa, synthetic construct; identical to SEQ ID NO: 8

SEQ ID NO: 201 - protein, 120 aa, synthetic construct
EVQLVESGGGLVKPGGSLRLSCAASGFTFSKYGVSWVRQAPGKGLEWVSTISSGGGYTHYVESVKGRFTISRDNAKNSLYLQMNSLRAEDTAVYYCARHPRDFSYAMDYWGQGTTVTVSS

SEQ ID NO: 202 - protein, 107 aa, synthetic construct; identical to SEQ ID NO: 8

SEQ ID NO: 203 - protein, 120 aa, synthetic construct; identical to SEQ ID NO: 21

SEQ ID NO: 204 - protein, 107 aa, synthetic construct; identical to SEQ ID NO: 16

SEQ ID NO: 205 - protein, 120 aa, synthetic construct
EVQLVESGGGLVKPGGSLRLSCAASGFTFSHYGMSWVRQAPGKGLEWVSTISSGGGYTHYAESVKGRFTISRDNAKNSLYLQMNSLRAEDTAVYYCARHPRDFSYAFDYWGQGTTVTVSS

SEQ ID NO: 206 - protein, 107 aa, synthetic construct; identical to SEQ ID NO: 24

SEQ ID NO: 207 - protein, 120 aa, synthetic construct; identical to SEQ ID NO: 23

SEQ ID NO: 208 - protein, 107 aa, synthetic construct; identical to SEQ ID NO: 24

SEQ ID NO: 209 - protein, 120 aa, synthetic construct
EVQLVESGGGLVKPGGSLRLSCAASGFTFSPYGMSWVRQAPGKGLEWVSTISSGGGYTYYAASVKGRFTISRDNAKNSLYLQMNSLRAEDTAVYYCARHPRDFSYAMDYWGQGTTVTVSS

SEQ ID NO: 210 - protein, 107 aa, synthetic construct; identical to SEQ ID NO: 2

SEQ ID NO: 211 - protein, 120 aa, synthetic construct
EVQLVESGGGLVKPGGSLRLSCAASGFTFSKYGTSWVRQAPGKGLEWVSTISSGGGYTHYAPSVKGRFTISRDNAKNSLYLQMNSLRAEDTAVYYCARHPRDFSYALDYWGQGTTVTVSS

SEQ ID NO: 212 - protein, 107 aa, synthetic construct; identical to SEQ ID NO: 24

SEQ ID NO: 213 - protein, 120 aa, synthetic construct
EVQLVESGGGLVKPGGSLRLSCAASGFTFSAYGASWVRQAPGKGLEWVSTISSGGGYTHYAGSVKGRFTISRDNAKNSLYLQMNSLRAEDTAVYYCARHPRDFSYAQDYWGQGTTVTVSS

SEQ ID NO: 214 - protein, 107 aa, synthetic construct; identical to SEQ ID NO: 8

SEQ ID NO: 215 - protein, 120 aa, synthetic construct
EVQLVESGGGLVKPGGSLRLSCAASGFTFSGYGMSWVRQAPGKGLEWVSTISSGGGYTHYVESVKGRFTISRDNAKNSLYLQMNSLRAEDTAVYYCARHPRDFSYALDYWGQGTTVTVSS

SEQ ID NO: 216 - protein, 107 aa, synthetic construct; identical to SEQ ID NO: 8

SEQ ID NO: 217 - protein, 120 aa, synthetic construct; identical to SEQ ID NO: 25

SEQ ID NO: 218 - protein, 107 aa, synthetic construct; identical to SEQ ID NO: 2

SEQ ID NO: 219 - protein, 120 aa, synthetic construct
EVQLVESGGGLVKPGGSLRLSCAASGFTFSAYGSSWVRQAPGKGLEWVSTISSGGGYTHYAPSVKGRFTISRDNAKNSLYLQMNSLRAEDTAVYYCARHPRDFSYAMDYWGQGTTVTVSS

SEQ ID NO: 220 - protein, 107 aa, synthetic construct; identical to SEQ ID NO: 2

SEQ ID NO: 221 - protein, 120 aa, synthetic construct
EVQLVESGGGLVKPGGSLRLSCAASGFTFSSYGQSWVRQAPGKGLEWVSTISSGGGYTHYAPSVKGRFTISRDNAKNSLYLQMNSLRAEDTAVYYCARHPRDFSYAMDYWGQGTTVTVSS

SEQ ID NO: 222 - protein, 107 aa, synthetic construct; identical to SEQ ID NO: 4

SEQ ID NO: 223 - protein, 120 aa, synthetic construct
EVQLVESGGGLVKPGGSLRLSCAASGFTFSHYGLNWVRQAPGKGLEWVSTISSGGGYTHYAPSVKGRFTISRDNAKNSLYLQMNSLRAEDTAVYYCARHPRDFSYANDYWGQGTTVTVSS

SEQ ID NO: 224 - protein, 107 aa, synthetic construct; identical to SEQ ID NO: 2

SEQ ID NO: 225 - protein, 120 aa, synthetic construct
EVQLVESGGGLVKPGGSLRLSCAASGFTFSSYGMNWVRQAPGKGLEWVSTISSGGGYTHYADSVKGRFTISRDNAKNSLYLQMNSLRAEDTAVYYCARHPRDFSYAQDYWGQGTTVTVSS

SEQ ID NO: 226 - protein, 107 aa, synthetic construct; identical to SEQ ID NO: 92

SEQ ID NO: 227 - protein, 120 aa, synthetic construct
EVQLVESGGGLVKPGGSLRLSCAASGFTFSKYGMSWVRQAPGKGLEWVSTISSGGGYTHYADSVKGRFTISRDNAKNSLYLQMNSLRAEDTAVYYCARHPRDFSYAMDYWGQGTTVTVSS

SEQ ID NO: 228 - protein, 107 aa, synthetic construct; identical to SEQ ID NO: 24

SEQ ID NO: 229 - protein, 120 aa, synthetic construct
EVQLVESGGGLVKPGGSLRLSCAASGFTFSVYGMSWVRQAPGKGLEWVSTISSGGGYTHYAPSVKGRFTISRDNAKNSLYLQMNSLRAEDTAVYYCARHPRDFSYAMDYWGQGTTVTVSS

SEQ ID NO: 230 - protein, 107 aa, synthetic construct; identical to SEQ ID NO: 2

SEQ ID NO: 231 - protein, 120 aa, synthetic construct
EVQLVESGGGLVKPGGSLRLSCAASGFTFSQYGMSWVRQAPGKGLEWVSTISSGGGYTHYVQSVKGRFTISRDNAKNSLYLQMNSLRAEDTAVYYCARHPRDFSYAQDYWGQGTTVTVSS

SEQ ID NO: 232 - protein, 107 aa, synthetic construct; identical to SEQ ID NO: 8

SEQ ID NO: 233 - protein, 120 aa, synthetic construct
EVQLVESGGGLVKPGGSLRLSCAASGFTFSHYGANWVRQAPGKGLEWVSTISSGGGYTHYATSVKGRFTISRDNAKNSLYLQMNSLRAEDTAVYYCARHPRDFSYAVDYWGQGTTVTVSS

SEQ ID NO: 234 - protein, 107 aa, synthetic construct
EIVMTQSPATLSVSPGERATLSCRASQDVGHYVAWYQQKPGQAPRLLIYWASTSATGIPARFSGSGSGTEFTLTISSLQSEDFAVYYCQQYNIYPWTFGQGTKVEIK

SEQ ID NO: 235 - protein, 120 aa, synthetic construct
EVQLVESGGGLVKPGGSLRLSCAASGFTFSQYGMSWVRQAPGKGLEWVSTISSGGGYTHYVTSVKGRFTISRDNAKNSLYLQMNSLRAEDTAVYYCARHPRDFSYAKDYWGQGTTVTVSS

SEQ ID NO: 236 - protein, 107 aa, synthetic construct; identical to SEQ ID NO: 2

SEQ ID NO: 237 - protein, 120 aa, synthetic construct
EVQLVESGGGLVKPGGSLRLSCAASGFTFSKYGMSWVRQAPGKGLEWVSTISSGGGYTHYVHSVKGRFTISRDNAKNSLYLQMNSLRAEDTAVYYCARHPRDFSYANDYWGQGTTVTVSS

SEQ ID NO: 238 - protein, 107 aa, synthetic construct; identical to SEQ ID NO: 6

SEQ ID NO: 239 - protein, 120 aa, synthetic construct
EVQLVESGGGLVKPGGSLRLSCAASGFTFSGYGASWVRQAPGKGLEWVSTISSGGGYTHYAPSVKGRFTISRDNAKNSLYLQMNSLRAEDTAVYYCARHPRDFSYAQDYWGQGTTVTVSS

SEQ ID NO: 240 - protein, 107 aa, synthetic construct; identical to SEQ ID NO: 4

SEQ ID NO: 241 - protein, 120 aa, synthetic construct
EVQLVESGGGLVKPGGSLRLSCAASGFTFSDYGISWVRQAPGKGLEWVSTISSGGGYTHYVQSVKGRFTISRDNAKNSLYLQMNSLRAEDTAVYYCARHPRDFSYAMDYWGQGTTVTVSS

SEQ ID NO: 242 - protein, 107 aa, synthetic construct; identical to SEQ ID NO: 8

SEQ ID NO: 243 - protein, 120 aa, synthetic construct
EVQLVESGGGLVKPGGSLRLSCAASGFTFSTYGTSWVRQAPGKGLEWVSTISSGGGYTHYAPSVKGRFTISRDNAKNSLYLQMNSLRAEDTAVYYCARHPRDFSYALDYWGQGTTVTVSS

SEQ ID NO: 244 - protein, 107 aa, synthetic construct; identical to SEQ ID NO: 24

SEQ ID NO: 245 - protein, 120 aa, synthetic construct
EVQLVESGGGLVKPGGSLRLSCAASGFTFSAYGLSWVRQAPGKGLEWVSTISSGGGYTHYAQSVKGRFTISRDNAKNSLYLQMNSLRAEDTAVYYCARHPRDFSYAFDYWGQGTTVTVSS

SEQ ID NO: 246 - protein, 107 aa, synthetic construct; identical to SEQ ID NO: 8

SEQ ID NO: 247 - protein, 120 aa, synthetic construct
EVQLVESGGGLVKPGGSLRLSCAASGFTFSSYGISWVRQAPGKGLEWVSTISSGGGYTHYAANVKGRFTISRDNAKNSLYLQMNSLRAEDTAVYYCARHPRDFSYAHDYWGQGTTVTVSS

SEQ ID NO: 248 - protein, 107 aa, synthetic construct; identical to SEQ ID NO: 4

SEQ ID NO: 249 - protein, 120 aa, synthetic construct
EVQLVESGGGLVKPGGSLRLSCAASGFTFSGYGMNWVRQAPGKGLEWVSTISSGGGYTHYVESVKGRFTISRDNAKNSLYLQMNSLRAEDTAVYYCARHPRDFSYAMDYWGQGTTVTVSS

SEQ ID NO: 250 - protein, 107 aa, synthetic construct; identical to SEQ ID NO: 8

SEQ ID NO: 251 - protein, 120 aa, synthetic construct
EVQLVESGGGLVKPGGSLRLSCAASGFTFSRYGQSWVRQAPGKGLEWVSTISSGGGYTHYAPSVKGRFTISRDNAKNSLYLQMNSLRAEDTAVYYCARHPRDFSYALDYWGQGTTVTVSS

SEQ ID NO: 252 - protein, 107 aa, synthetic construct; identical to SEQ ID NO: 6

SEQ ID NO: 253 - protein, 120 aa, synthetic construct
EVQLVESGGGLVKPGGSLRLSCAASGFTFSGYGVSWVRQAPGKGLEWVSTISSGGGYTHYVDSVKGRFTISRDNAKNSLYLQMNSLRAEDTAVYYCARHPRDFSYALDYWGQGTTVTVSS

SEQ ID NO: 254 - protein, 107 aa, synthetic construct; identical to SEQ ID NO: 16

SEQ ID NO: 255 - protein, 120 aa, synthetic construct
EVQLVESGGGLVKPGGSLRLSCAASGFTFSNYGVSWVRQAPGKGLEWVSTISSGGGYTHYAPSVKGRFTISRDNAKNSLYLQMNSLRAEDTAVYYCARHPRDFSYAQDYWGQGTTVTVSS

SEQ ID NO: 256 - protein, 107 aa, synthetic construct; identical to SEQ ID NO: 6

SEQ ID NO: 257 - protein, 120 aa, synthetic construct
EVQLVESGGGLVKPGGSLRLSCAASGFTFSAYGMSWVRQAPGKGLEWVSTISSGGGYTHYVESVKGRFTISRDNAKNSLYLQMNSLRAEDTAVYYCARHPRDFSYAMDYWGQGTTVTVSS

SEQ ID NO: 258 - protein, 107 aa, synthetic construct; identical to SEQ ID NO: 2

SEQ ID NO: 259 - protein, 120 aa, synthetic construct
EVQLVESGGGLVKPGGSLRLSCAASGFTFSAYGVSWVRQAPGKGLEWVSTISSGGGYTYYVESVKGRFTISRDNAKNSLYLQMNSLRAEDTAVYYCARHPRDFSYAMDYWGQGTTVTVSS

SEQ ID NO: 260 - protein, 107 aa, synthetic construct; identical to SEQ ID NO: 16

SEQ ID NO: 261 - protein, 120 aa, synthetic construct
EVQLVESGGGLVKPGGSLRLSCAASGFTFSNYGMNWVRQAPGKGLEWVSTISSGGGYTHYAPSVKGRFTISRDNAKNSLYLQMNSLRAEDTAVYYCARHPRDFSYAMDYWGQGTTVTVSS

SEQ ID NO: 262 - protein, 107 aa, synthetic construct; identical to SEQ ID NO: 2

SEQ ID NO: 263 - protein, 120 aa, synthetic construct
EVQLVESGGGLVKPGGSLRLSCAASGFTFSHYGLSWVRQAPGKGLEWVSTISSGGGYTHYVDSVKGRFTISRDNAKNSLYLQMNSLRAEDTAVYYCARHPRDFSYANDYWGQGTTVTVSS

SEQ ID NO: 264 - protein, 107 aa, synthetic construct; identical to SEQ ID NO: 4

SEQ ID NO: 265 - protein, 120 aa, synthetic construct
EVQLVESGGGLVKPGGSZRLSCAASGFTFSFYGMSWVRQAPGKGLEWVSTISSGGGYTYYAESVKGRFTISRDNAKNSLYLQMNSLRAEDTAVYYCARHPRDFSYAIDYWGQGTTVTVSS

SEQ ID NO: 266 - protein, 107 aa, synthetic construct; identical to SEQ ID NO: 6

SEQ ID NO: 267 - protein, 120 aa, synthetic construct; identical to SEQ ID NO: 27

SEQ ID NO: 268 - protein, 107 aa, synthetic construct; identical to SEQ ID NO: 2

SEQ ID NO: 269 - protein, 120 aa, synthetic construct; identical to SEQ ID NO: 55

SEQ ID NO: 270 - protein, 107 aa, synthetic construct; identical to SEQ ID NO: 8

SEQ ID NO: 271 - protein, 120 aa, synthetic construct
EVQLVESGGGLVKPGGSLRLSCAASGFTFSSYGVSWVRQAPGKGLEWVSTISSGGGYTHYVESVKGRFTISRDNAKNSLYLQMNSLRAEDTAVYYCARHPRDFSYAQDYWGQGTTVTVSS

SEQ ID NO: 272 - protein, 107 aa, synthetic construct; identical to SEQ ID NO: 8

SEQ ID NO: 273 - protein, 120 aa, synthetic construct
EVQLVESGGGLVKPGGSLRLSCAASGFTFSTYGHSWVRQAPGKGLEWVSTISSGGGYTHYVASVKGRFTISRDNAKNSLYLQMNSLRAEDTAVYYCARHPRDFSYAMDYWGQGTTVTVSS

SEQ ID NO: 274 - protein, 107 aa, synthetic construct
EIVMTQSPATLSVSPGERATLSCRASQDVGHYVAWYQQKPGQAPRLLIYWASTRHTGIPARFSGSGSGTEFTLTISSLQSEDFAVYYCQQYNIYPWTFGQGIKVEIK

SEQ ID NO: 275 - protein, 120 aa, synthetic construct
EVQLVESGGGLVKPGGSLRLSCAASGFTFSSYGISWVRQAPGKGLEWVSTISSGGGYTYYVDSVKGRFTISRDNAKNSLYLQMNSLRAEDTAVYYCARHPRDFSYALDYWGQGTTVTVSS

SEQ ID NO: 276 - protein, 107 aa, synthetic construct; identical to SEQ ID NO: 2

SEQ ID NO: 277 - protein, 120 aa, synthetic construct; identical to SEQ ID NO: 29

SEQ ID NO: 278 - protein, 107 aa, synthetic construct; identical to SEQ ID NO: 2

SEQ ID NO: 279 - protein, 120 aa, synthetic construct
EVQLVESGGGLVKPGGSLRLSCAASGFTFSRYGVSWVRQAPGKGLEWVSTISSGGGYTHYAESVKGRFTISRDNAKNSLYLQMNSLRAEDTAVYYCARHPRDFSYAMDYWGQGTTVTVSS

SEQ ID NO: 280 - protein, 107 aa, synthetic construct; identical to SEQ ID NO: 8

SEQ ID NO: 281 - protein, 120 aa, synthetic construct
EVQLVESGGGLVKPGGSLRLSCAASGFTFSNYGTSWVRQAPGKGLEWVSTISSGGGYTHYVTSVKGRFTISRDNAKNSLYLQMNSLRAEDTAVYYCARHPRDFSYAMDYWGQGTTVTVSS

SEQ ID NO: 282 - protein, 107 aa, synthetic construct; identical to SEQ ID NO: 2

SEQ ID NO: 283 - protein, 120 aa, synthetic construct
EVQLVESGGGLVKPGGSLRLSCAASGFTFSHYGVSWVRQAPGKGLEWVSTISSGGGYTHYAESVKGRFTISRDNAKNSLYLQMNSLRAEDTAVYYCARHPRDFSYAQDYWGQGTTVTVSS

SEQ ID NO: 284 - protein, 107 aa, synthetic construct; identical to SEQ ID NO: 16

SEQ ID NO: 285 - protein, 120 aa, synthetic construct
EVQLVESGGGLVKPGGSLRLSCAASGFTFSTYGMNWVRQAPGKGLEWVSTISSGGGYTHYAESVKGRFTISRDNAKNSLYLQMNSLRAEDTAVYYCARHPRDFSYAVDYWGQGTTVTVSS

SEQ ID NO: 286 - protein, 107 aa, synthetic construct; identical to SEQ ID NO: 8

SEQ ID NO: 287 - protein, 120 aa, synthetic construct
EVQLVESGGGLVKPGGSLRLSCAASGFTFSSYGISWVRQAPGKGLEWVSTISSGGGYTHYAASVKGRFTISRDNAKNSLYLQMNSLRAEDTAVYYCARHPRDFSYALDYWGQGTTVTVSS

SEQ ID NO: 288 - protein, 107 aa, synthetic construct; identical to SEQ ID NO: 4

SEQ ID NO: 289 - protein, 120 aa, synthetic construct
EVQLVESGGGLVKPGGSLRLSCAASGFTFSNYGMSWVRQAPGKGLEWVSTISSGGGYTHYAPSVKGRFTISRDNAKNSLYLQMNSLRAEDTAVYYCARHPRDFSYAMDYWGQGTTVTVSS

SEQ ID NO: 290 - protein, 107 aa, synthetic construct; identical to SEQ ID NO: 8

SEQ ID NO: 291 - protein, 120 aa, synthetic construct
EVQLVESGGGLVKPGGSLRLSCAASGFTFSKYGMSWVRQAPGKGLEWVSTISSGGGYTHYVTSVKGRFTISRDNAKNSLYLQMNSLRAEDTAVYYCARHPRDFSYAMDYWGQGTTVTVSS

SEQ ID NO: 292 - protein, 107 aa, synthetic construct; identical to SEQ ID NO: 8

SEQ ID NO: 293 - protein, 120 aa, synthetic construct
EVQLVESGGGLVKPGGSLRLSCAASGFTFSGYGMSWVRQAPGKGLEWVSTISSGGGYTHYVTSVKGRFTISRDNAKNSLYLQMNSLRAEDTAVYYCARHPRDFSYALDYWGQGTTVTVSS

SEQ ID NO: 294 - protein, 107 aa, synthetic construct; identical to SEQ ID NO: 16

SEQ ID NO: 295 - protein, 120 aa, synthetic construct; identical to SEQ ID NO: 31

SEQ ID NO: 296 - protein, 107 aa, synthetic construct; identical to SEQ ID NO: 16

SEQ ID NO: 297 - protein, 120 aa, synthetic construct
EVQLVESGGGLVKPGGSLRLSCAASGFTFSVYGISWVRQAPGKGLEWVSTISSGGGYTHYAPSVKGRFTISRDNAKNSLYLQMNSLRAEDTAVYYCARHPRDFSYAMDYWGQGTTVTVSS

SEQ ID NO: 298 - protein, 107 aa, synthetic construct; identical to SEQ ID NO: 2

SEQ ID NO: 299 - protein, 120 aa, synthetic construct
EVQLVESGGGLVKPGGSLRLSCAASGFTFSRYGHSWVRQAPGKGLEWVSTISSGGGYTYYAESVKGRFTISRDNAKNSLYLQMNSLRAEDTAVYYCARHPRDFSYAHDYWGQGTTVTVSS

SEQ ID NO: 300 - protein, 107 aa, synthetic construct; identical to SEQ ID NO: 92

SEQ ID NO: 301 - protein, 120 aa, synthetic construct
EVQLVESGGGLVKPGGSLRLSCAASGFTFSAYGTSWVRQAPGKGLEWVSTISSGGGYTHYVHSVKGRFTISRDNAKNSLYLQMNSLRAEDTAVYYCARHPRDFSYAMDYWGQGTTVTVSS

SEQ ID NO: 302 - protein, 107 aa, synthetic construct; identical to SEQ ID NO: 4

SEQ ID NO: 303 - protein, 120 aa, synthetic construct
EVQLVESGGGLVKPGGSLRLSCAASGFTFSSYGMSWVRQAPGKGLEWVSTISSGGGYTHYVQSVKGRFTISRDNAKNSLYLQMNSLRAEDTAVYYCARHPRDFSYAMDYWGQGTTVTVSS

SEQ ID NO: 304 - protein, 107 aa, synthetic construct; identical to SEQ ID NO: 8

SEQ ID NO: 305 - protein, 10 aa, synthetic construct
GFTFSQYGHS

SEQ ID NO: 306 - protein, 12 aa, synthetic construct
TISSGGGYTHYA

SEQ ID NO: 307 - protein, 13 aa, synthetic construct
ARHPRDFSYANDY

SEQ ID NO: 308 - protein, 11 aa, synthetic construct
RASQDVGHYLA

SEQ ID NO: 309 - protein, 7 aa, synthetic construct
WASTRAT

SEQ ID NO: 310 - protein, 9 aa, synthetic construct
QQYNIYPWT

SEQ ID NO: 311 - protein, 10 aa, synthetic construct
GFTFSTYGVS

SEQ ID NO: 312 - protein, 12 aa, synthetic construct
TISSGGGYTHYA

SEQ ID NO: 313 - protein, 13 aa, synthetic construct
ARHPRDFSYALDY

SEQ ID NO: 314 - protein, 11 aa, synthetic construct
RASQDVGHYLA

SEQ ID NO: 315 - protein, 7 aa, synthetic construct
WASTRAT

SEQ ID NO: 316 - protein, 9 aa, synthetic construct
QQYNIYPWT

SEQ ID NO: 317 - protein, 10 aa, synthetic construct
GFTFSGYGLS

SEQ ID NO: 318 - protein, 12 aa, synthetic construct
TISSGGGYTHYA

SEQ ID NO: 319 - protein, 13 aa, synthetic construct
ARHPRDFSYAQDY

SEQ ID NO: 320 - protein, 11 aa, synthetic construct
KASQDVGHYVA

SEQ ID NO: 321 - protein, 7 aa, synthetic construct
WASTRAT

SEQ ID NO: 322 - protein, 9 aa, synthetic construct
QQYNIYPWT

SEQ ID NO: 323 - protein, 10 aa, synthetic construct
GFTFSHYGVS

SEQ ID NO: 324 - protein, 12 aa, synthetic construct
TISSGGGYTHYA

SEQ ID NO: 325 - protein, 13 aa, synthetic construct
ARHPRDFSYAQDY

SEQ ID NO: 326 - protein, 11 aa, synthetic construct
KASQDVGHYVA

SEQ ID NO: 327 - protein, 7 aa, synthetic construct
WASTRHT

SEQ ID NO: 328 - protein, 9 aa, synthetic construct
QQYNIYPWT

SEQ ID NO: 329 - protein, 10 aa, synthetic construct
GFTFSSYGHS

SEQ ID NO: 330 - protein, 12 aa, synthetic construct
TISSGGGYTHYA

SEQ ID NO: 331 - protein, 13 aa, synthetic construct
ARHPRDFSYAHDY

SEQ ID NO: 332 - protein, 11 aa, synthetic construct
RASQDVGHYVA

SEQ ID NO: 333 - protein, 7 aa, synthetic construct
WASTRAT

SEQ ID NO: 334 - protein, 9 aa, synthetic construct
QQYNIYPWT

SEQ ID NO: 335 - protein, 10 aa, synthetic construct
GFTFSQYGHS

SEQ ID NO: 336 - protein, 12 aa, synthetic construct
TISSGGGYTHYA

SEQ ID NO: 337 - protein, 13 aa, synthetic construct
ARHPRDFSYAQDY

SEQ ID NO: 338 - protein, 11 aa, synthetic construct
KASQDVGHYLA

SEQ ID NO: 339 - protein, 7 aa, synthetic construct
WASTRAT

SEQ ID NO: 340 - protein, 9 aa, synthetic construct
QQYNIYPWT

SEQ ID NO: 341 - protein, 10 aa, synthetic construct
GFTFSKYGTS

SEQ ID NO: 342 - protein, 12 aa, synthetic construct
TISSGGGYTHYA

SEQ ID NO: 343 - protein, 13 aa, synthetic construct
ARHPRDFSYAQDY

SEQ ID NO: 344 - protein, 11 aa, synthetic construct
RASQDVGHYVA

SEQ ID NO: 345 - protein, 7 aa, synthetic construct
WASTRAT

SEQ ID NO: 346 - protein, 9 aa, synthetic construct
QQYNIYPWT

SEQ ID NO: 347 - protein, 10 aa, synthetic construct
GFTFSHYGVS

SEQ ID NO: 348 - protein, 12 aa, synthetic construct
TISSGGGYTHYV

SEQ ID NO: 349 - protein, 13 aa, synthetic construct
ARHPRDFSYALDY

SEQ ID NO: 350 - protein, 11 aa, synthetic construct
KASQDVGHYVA

SEQ ID NO: 351 - protein, 7 aa, synthetic construct
WASTRAT

SEQ ID NO: 352 - protein, 9 aa, synthetic construct
QQYNIYPWA

SEQ ID NO: 353 - protein, 10 aa, synthetic construct
GFTFSVYGLS

SEQ ID NO: 354 - protein, 12 aa, synthetic construct
TISSGGGYTQYA

SEQ ID NO: 355 - protein, 13 aa, synthetic construct
ARHPRDFSYAFDY

SEQ ID NO: 356 - protein, 11 aa, synthetic construct
KASQDVGHYLA

SEQ ID NO: 357 - protein, 7 aa, synthetic construct
WASTRAT

SEQ ID NO: 358 - protein, 9 aa, synthetic construct
QQYNIYPWT

SEQ ID NO: 359 - protein, 10 aa, synthetic construct
GFTFSAYGTS

SEQ ID NO: 360 - protein, 12 aa, synthetic construct
TISSGGGYTYYA

SEQ ID NO: 361 - protein, 13 aa, synthetic construct
ARHPRDFSYAIDY

SEQ ID NO: 362 - protein, 11 aa, synthetic construct
RASQDVGHYLA

SEQ ID NO: 363 - protein, 7 aa, synthetic construct
WASTRAT

SEQ ID NO: 364 - protein, 9 aa, synthetic construct
QQYNIYPWT

SEQ ID NO: 365 - protein, 10 aa, synthetic construct
GFTFSSYGIS

SEQ ID NO: 366 - protein, 12 aa, synthetic construct
TISSGGGYTHYA

SEQ ID NO: 367 - protein, 13 aa, synthetic construct
ARHPRDFSYALDY

SEQ ID NO: 368 - protein, 11 aa, synthetic construct
KASQDVGHYLA

SEQ ID NO: 369 - protein, 7 aa, synthetic construct
WASTRAT

SEQ ID NO: 370 - protein, 9 aa, synthetic construct
QQYNIYPWT

SEQ ID NO: 371 - protein, 10 aa, synthetic construct
GFTFSQYGHS

SEQ ID NO: 372 - protein, 12 aa, synthetic construct
TISSGGGYTHYA

SEQ ID NO: 373 - protein, 13 aa, synthetic construct
ARHPRDFSYAQDY

SEQ ID NO: 374 - protein, 11 aa, synthetic construct
KASQDVGHYLA

SEQ ID NO: 375 - protein, 7 aa, synthetic construct
WASTRHT

SEQ ID NO: 376 - protein, 9 aa, synthetic construct
QQYNIYPWT

SEQ ID NO: 377 - protein, 10 aa, synthetic construct
GFTFSHYGTS

SEQ ID NO: 378 - protein, 12 aa, synthetic construct
TISSGGGYTHYA

SEQ ID NO: 379 - protein, 13 aa, synthetic construct
ARHPRDFSYAIDY

SEQ ID NO: 380 - protein, 11 aa, synthetic construct
KASQDVGHYVA

SEQ ID NO: 381 - protein, 7 aa, synthetic construct
WASTRAT

SEQ ID NO: 382 - protein, 9 aa, synthetic construct
QQYNIYPWT

SEQ ID NO: 383 - protein, 10 aa, synthetic construct
GFTFSKYGLS

SEQ ID NO: 384 - protein, 12 aa, synthetic construct
TISSGGGYTHYV

SEQ ID NO: 385 - protein, 13 aa, synthetic construct
ARHPRDFSYASDY

SEQ ID NO: 386 - protein, 11 aa, synthetic construct
KASQDVGHYVA

SEQ ID NO: 387 - protein, 7 aa, synthetic construct
WASTRAT

SEQ ID NO: 388 - protein, 9 aa, synthetic construct
QQYNIYPWT

SEQ ID NO: 389 - protein, 10 aa, synthetic construct
GFTFSTYGAS

SEQ ID NO: 390 - protein, 12 aa, synthetic construct
TISSGGGYTHYA

SEQ ID NO: 391 - protein, 13 aa, synthetic construct
ARHPRDFSYALDY

SEQ ID NO: 392 - protein, 11 aa, synthetic construct
KASQDVGHYVA

SEQ ID NO: 393 - protein, 7 aa, synthetic construct
WASTRAT

SEQ ID NO: 394 - protein, 9 aa, synthetic construct
QQYNIYPWT

SEQ ID NO: 395 - protein, 10 aa, synthetic construct
GFTFSAYGSS

SEQ ID NO: 396 - protein, 12 aa, synthetic construct
TISSGGGYTHYA

SEQ ID NO: 397 - protein, 13 aa, synthetic construct
ARHPRDFSYAHDY

SEQ ID NO: 398 - protein, 11 aa, synthetic construct
KASQDVGHYVA

SEQ ID NO: 399 - protein, 7 aa, synthetic construct
WASTRHT

SEQ ID NO: 400 - protein, 9 aa, synthetic construct
QQYNIYPWT

(19) United States
(12) Patent Application Publication
(10) Pub. No.: US 2026/0209348 A1
(43) Pub. Date: Jul. 23, 2026

(54) ANTI-ROR-2 ANTIBODIES AND METHODS OF USE

(71) Applicant: The Regents of the University of California, Oakland, CA (US)

(72) Inventors: Thomas J. Kipps, San Diego, CA (US); George F. Widhopf, II, San Diego, CA (US)

(21) Appl. No.: 19/144,217

(22) PCT Filed: Dec. 29, 2023
(86) PCT No.: PCT/US23/86552

§ 371 Date: Jun. 27, 2025

Related U.S. Application Data
(60) Provisional application No. 63/478,067, filed on Dec. 30, 2022.

(51) Int. Cl. C07K 16/28; A61K 40/31; A61K 40/42; A61K 45/06; G01N 33/68

(52) U.S. Cl. CPC C07K 16/2803; A61K 40/31; A61K 40/4202; A61K 45/06; G01N 33/6854; A61K 2239/13; A61K 2239/21; C07K 2317/24; C07K 2317/55; C07K 2317/565; C07K 2317/567; C07K 2317/622; C07K 2317/92; G01N 2333/705

(57) ABSTRACT

Provided herein are, inter alia, antibodies (e.g., humanized antibodies, monoclonal antibodies), antibody fragments (e.g., scFvs) and antibody compositions (e.g., chimeric antigen receptors, bispecific antibodies), which bind human tyrosine kinase-like orphan receptor 2 (ROR2) with high efficiency and specificity and are, inter alia, useful for diagnosing and treating cancer and other ROR2-related diseases.

### CROSS-REFERENCES TO RELATED APPLICATIONS

This application claims the benefit of U.S. Provisional Application No. 63/478,067, filed Dec. 30, 2022, which is incorporated herein by reference in its entirety and for all purposes.

### BACKGROUND

Receptor tyrosine kinase-like orphan receptor 2 (ROR2) is a developmentally restricted receptor for certain Wingless-related integration site (Wnt) factors, such as Wnt5a. Human ROR2 is a 943 amino acid single-pass type I membrane protein with a calculated molecular weight of 104.8 kDa. It is highly conserved across several species with a 92% amino acid identity between the mouse and human proteins. ROR2 can repress transcription of Wnt target genes and modulate Wnt signaling by sequestering canonical Wnt ligands, thereby serving as a tumor suppressor in different cell contexts. Consistent with it primarily serving a function(s) in embryogenesis. expression of ROR2 attenuates during embryonic/fetal development and most normal post-partem tissues do not express ROR2. However, ROR2 is expressed by a variety of cancers (e.g., breast, ovarian, pancreatic, cervical, gastric, renal, head and neck, bone, skin, soft tissue, or prostate cancer), in which it may promote tumo progression, metastases, and/or self-renewal. Therefore, there is a need for antibodies. antibody fragments, bispecific antibodies and chimeric antigen receptors that specifically target human ROR2, inhibit its function and thereby serve as effective therapeutics and diagnostics. The compositions and methods provided herein address these and other needs in the art.

### BRIEF SUMMARY OF THE INVENTION

In an aspect is provided, a method of identifying an anti-ROR2 antibody. The method includes: (i) contacting an antibody with a first ROR2 polypeptide including a histidine at a position corresponding to position 349 of SEQ ID NO:34: (ii) detecting the antibody binding to the first ROR2 polypeptide; (iii) contacting the antibody with a second ROR2 polypeptide not including a histidine at a position corresponding to position 349 of SEQ ID NO:34; and (iv) detecting the antibody not binding to the second ROR2 polypeptide, thereby identifying an anti-ROR2 antibody.

In an aspect is provided, a method of identifying an anti-ROR2 antibody. The method includes: (i) contacting an antibody with a first ROR2 polypeptide including an aspartic acid at a position corresponding to position 354 of SEQ ID NO:34: (ii) detecting the antibody binding to the first ROR2 polypeptide; (iii) contacting the antibody with a second ROR2 polypeptide not including an aspartic acid at a position corresponding to position 354 of SEQ ID NO:34; and (iv) detecting the antibody not binding to the second ROR2 polypeptide, thereby identifying an anti-ROR2 antibody.

In an aspect is provided, a method of identifying an anti-ROR2 antibody. The method includes: (i) contacting an antibody with a first ROR2 polypeptide including a methionine at a position corresponding to position 386 of SEQ ID NO:34; (ii) detecting the antibody binding to the first ROR2 polypeptide; (iii) contacting the antibody with a second ROR2 polypeptide not including a methionine at a position corresponding to position 386 of SEQ ID NO:34; and (iv) detecting the antibody not binding to the second ROR2 polypeptide, thereby identifying an anti-ROR2 antibody.

In an aspect is provided, an anti-tyrosine kinase-like orphan receptor 2 (ROR2) antibody including a heavy chain variable domain and a light chain variable domain, wherein the heavy chain variable domain includes the sequence of SEQ ID NO: 1 and wherein the light chain variable domain includes the sequence of SEQ ID NO: 2; wherein the heavy chain variable domain includes the sequence of SEQ ID NO: 3 and wherein the light chain variable domain includes the sequence of SEQ ID NO: 4; wherein the heavy chain variable domain includes the sequence of SEQ ID NO: 5 and wherein the light chain variable domain includes the sequence of SEQ ID NO: 6: wherein the heavy chain variable domain includes the sequence of SEQ ID NO: 7 and wherein the light chain variable domain includes the sequence of SEQ ID NO: 8; wherein the heavy chain variable domain includes the sequence of SEQ ID NO: 9 and wherein the light chain variable domain includes the sequence of SEQ ID NO: 10; wherein the heavy chain variable domain includes the sequence of SEQ ID NO: 11 and wherein the light chain variable domain includes the sequence of SEQ ID NO: 12: wherein the heavy chain variable domain includes the sequence of SEQ ID NO: 13 and wherein the light chain variable domain includes the sequence of SEQ ID NO: 14; wherein the heavy chain variable domain includes the sequence of SEQ ID NO: 15 and wherein the light chain variable domain includes the sequence of SEQ ID NO: 16; wherein the heavy chain variable domain includes the sequence of SEQ ID NO: 17 and wherein the light chain variable domain includes the sequence of SEQ ID NO: 18; wherein the heavy chain variable domain includes the sequence of SEQ ID NO: 19 and wherein the light chain variable domain includes the sequence of SEQ ID NO: 20; wherein the heavy chain variable domain includes the sequence of SEQ ID NO: 21 and wherein the light chain variable domain includes the sequence of SEQ ID NO: 22; wherein the heavy chain variable domain includes the sequence of SEQ ID NO: 23 and wherein the light chain variable domain includes the sequence of SEQ ID NO: 24: wherein the heavy chain variable domain includes the sequence of SEQ ID NO: 25 and wherein the light chain variable domain includes the sequence of SEQ ID NO: 26: wherein the heavy chain variable domain includes the sequence of SEQ ID NO: 27 and wherein the light chain variable domain includes the sequence of SEQ ID NO: 28; wherein the heavy chain variable domain includes the sequence of SEQ ID NO: 29 and wherein the light chain variable domain includes the sequence of SEQ ID NO: 30; or wherein the heavy chain variable domain includes the sequence of SEQ ID NO: 31 and wherein the light chain variable domain includes the sequence of SEQ ID NO: 32.

In an aspect is provided a method of treating cancer in a subject in need thereof, the method including administering to a subject a therapeutically effective amount of an antibody as provided herein including embodiments thereof.

In an aspect is provided a method of inhibiting metastasis of a ROR2-expressing cancer in a subject in need thereof, the method including administering to a subject a therapeutically effective amount of an antibody as provided herein including embodiments thereof.

In an aspect is provided a chimeric antigen receptor including (i) an antibody region including any one of the light chain variable domain and heavy chain variable domain pairings provided herein including embodiments thereof; and (ii) a transmembrane domain.

In an aspect is provided an anti-ROR2 antibody capable of binding the same epitope as an antibody provided herein including embodiments thereof.

In another aspect is provided anti-tyrosine kinase-like orphan receptor 2 (ROR2) antibody including: (i) a CDR H1 including an amino acid sequence of formula: G-F-T-F-S-X1-Y-G-X2-X3 (I), wherein X1 is A, D, E, F, G, H, K, N, P, Q, R, S, T, or V, X2 is A, H, I, L, M, Q, S, T, or V, X3 is S or N, and wherein if X2 is M and X3 is S X1 is not N; (ii) a CDR H2 including an amino acid sequence of formula: X4-I-S-S-G-G-G-Y-T-X5-Y-X6 (II), wherein X4 is T or S, X5 is H or Y, X6 is V or A; (iii) a CDR H3 including an amino acid sequence of formula: A-R-H-P-R-D-F-S-Y-A-X7-D-Y (III); wherein X7 is A, F, H, I, K, L, M, N, Q, S, T, or V; (iv) a CDR L1 including an amino acid sequence of formula: Q-D-V-G-H-Y (IV); (v) a CDR L2 including an amino acid sequence of formula: W-A-S (V); and (vi) a CDR L3 including the sequence of SEQ ID NO:53

In another aspect is provided an anti-tyrosine kinase-like orphan receptor 2 (ROR2) antibody including: (i) a CDR H1 including an amino acid sequence of formula: G-F-T-F-S-X1-Y-G-X2-X3 (I), wherein X1 is A, D, E, F, G, H, I, K, L, N, P, Q, R, S, T, V, W, or Y, X2 is A, E, F, G, H, I, K, L, M, P, Q, R, S, T, V, W, or Y, X3 is S or N, and wherein if X2 is M and X3 is S X1 is not N; (ii) a CDR H2 including an amino acid sequence of formula: X4-I-S-S-G-G-G-Y-T-X5-Y-X6 (II), wherein X4 is T or S, X5 is H or Y, X6 is V or A, and wherein if X5 is H and X6 is V X4 is not T; (iii) a CDR H3 including an amino acid sequence of formula: D-F-S-Y-A-X7-D-Y-W-G (III); wherein X7 is A, D, E, F, G, H, I, K, L, M, P, Q, R, S, T, V, W, or Y; (iv) a CDR L1 including an amino acid sequence of formula: X8-A-S-Q-D-V-G-H-Y-X9-A (IV) wherein X8 is K or R, X9 is V or L, and wherein if X8 is K X9 is not V; (v) a CDR L2 including an amino acid sequence of formula: W-A-S-T-R-X10-T (V) wherein X10 is H or A, and wherein if X8 is K and X9 is V X10 is not H; and (vi) a CDR L3 including the sequence of SEQ ID NO:53.

In another aspect is provided an anti-tyrosine kinase-like orphan receptor 2 (ROR2) antibody including: (i) a CDR H1 including an amino acid sequence of formula: G-F-T-F-S-X1-Y-G-X2-X3 (I), wherein X1 is A, D, E, F, G, H, K, N, P, Q, R, S, T, or V, X2 is A, , , H, I, , L, M, , Q, , S, T, or V, X3 is S or N, and wherein if X2 is M and X3 is S X1 is not N; (ii) a CDR H2 including an amino acid sequence of formula: X4-I-S-S-G-G-G-Y-T-X5-Y-X6 (II), wherein X4 is T or S, X5 is H or Y, X6 is V or A, and wherein if X5 is H and X6 is V X4 is not T; (iii) a CDR H3 including an amino acid sequence of formula: A-R-H-P-R-D-F-S-Y-A-X7-D-Y (III); wherein X7 is A, F, H, I, K, L, M, N, P, Q, S, T, or V; (iv) a CDR L1 including an amino acid sequence of formula: Q-D-V-G-H-Y; (v) a CDR L2 including an amino acid sequence of formula: W-A-S; and (vi) a CDR L3 including the sequence of SEQ ID NO:53.

In another aspect is provided a method of treating cancer in a subject in need thereof, said method including administering to a subject a therapeutically effective amount of an antibody provided herein including embodiments thereof.

In another aspect is provided a method of detecting a ROR2 expressing cell, said method including (i) contacting a ROR2-expressing cell with an antibody provided herein including embodiments thereof; and (ii) detecting binding of said antibody to a ROR2 protein expressed by said cell.

In another aspect is provided a method of delivering a therapeutic agent to a ROR2 expressing cell, said method including contacting a ROR2 expressing cell with an antibody provided herein including embodiments thereof, wherein said antibody is attached to a therapeutic agent.

In another aspect is provided a method of inhibiting migration of a ROR2-expressing cell, said method including contacting a ROR2 expressing cell with an antibody provided herein including embodiments thereof.

In another aspect is provided an anti-ROR2 antibody, wherein said anti-ROR2 antibody binds the same epitope as an anti-ROR2 antibody provided herein including embodiments thereof.

In another aspect is provided a chimeric antigen receptor including (i) an anti-ROR2 antibody provided herein including embodiments thereof and (ii) a transmembrane domain.

In an aspect is provided a method of treating cancer in a subject in need thereof said method including, administering a therapeutically effective amount of a chimeric antigen receptor provided herein including embodiments thereof to said subject.

### BRIEF DESCRIPTION OF THE DRAWINGS

FIG. 1 represents a comparison of extracellular regions of a human and a mouse Receptor Tyrosine Kinase Like Orphan Receptor 2 (ROR2). The alignment of amino acid sequences of the extracellular region of the human (upper sequence) and the mouse (lower sequence) ROR2 are shown. Dots indicate homology at that position, whereas differences are designated by single letter amino acid codons. Ig-like, cysteine rich domain (CRD) and Kringle domains are labeled and indicated by lines above the sequence.

FIGS. 2A-2C represent identification of binding regions of an anti-human ROR2 mAb by assessment of binding to chimeric human/mouse recombinant ROR2 protein. FIG. 2A shows schematics of the chimeric constructs of the extracellular portion of the ROR2 used to map the binding region of each of the ROR2 mAb. Dark regions of each construct indicate the human ROR2 and hatched portions regions of the mouse ROR2. h1-111 and h1-160 refer to first 111 and 160 amino acids of the human ROR2, respectively. hCRD and hKringle contain the cysteine rich domain and Kringle domain of the human ROR2, respectively. FIG. 2B illustrates the alignment of the protein sequences of the Kringle domain of human and mouse ROR2. Numbers above the sequences designate the position of the amino acid residue below. A dot in the sequence of mROR2 indicates sequence homology with hROR2 at that position. A letter in the mROR2 sequence indicates the amino acid that differs from hROR2 at that position. Recombinant human ROR2 proteins in which one amino acid that differs between the human and mouse ROR2 within the Kringle domain was replaced with the corresponding amino acid of mouse ROR2 are boxed and designated in the key as the amino acid in hROR2, the amino acid position, and the amino acid present in mROR2 that was substituted (T327M). FIG. 2C illustrates that each recombinant protein was transferred onto nylon membrane, probed with a c6E6 mAb, and detected with an anti-human IgG antibody conjugated with horradish peroxidase. The c6E6 mAb binds to ROR2 recombinant proteins that contain the human Kringle domain. The c6E6 binding is lost with the substitutions made for amino acids at positions 349 and 354.

FIG. 3 represents identification of the binding regions of the humanized anti-human ROR2 mAb by assessment of binding to chimeric human/house recombinant ROR2 protein. The recombinant human/mouse ROR2 proteins depicted in FIG. 2A were transferred onto nylon membrane, probed with the h6E6 mAb, and detected with an anti-human IgG antibody conjugated with horse radish peroxidase. The key on the right designates the position of each recombinant protein that is arrayed in two columns of six. Underlined number above each pair of columns designates a name of the h6E6 mAb used for that membrane. Each membrane was incubated individually with primary and secondary antibodies and developed and imaged together. Each of the sixteen h6E6 mAb has the same binding pattern as the chimeric 6E6 mAb, which is the parental mouse variable regions fused to human IgG1. Each mAb bind to the ROR2 recombinant proteins that contain the human Kringle domain, and binding is abrogated with the substitutions made for amino acids at positions 349 and 354, indicating that specific binding to the human ROR2 was not altered by humanization.

FIG. 4 represents identification of the binding regions of the anti-human ROR2 mAb by assessment of binding to the chimeric human/mouse recombinant ROR2 protein.

FIGS. 5A-5B represent an alignment of an immunoglobulin heavy chain variable region gene (IGHV) or an immunoglobulin kappa variable cluster (IGKV) amino acid sequences of sixteen humanized anti-human ROR2 hybridomas. For each alignment, the uppermost sequence depicts the amino acid sequence of the heavy (FIG. 5A) or light (FIG. 5B) chain variable region beginning at the first codon of first framework region and ending with the last codon of the fourth framework region. The amino acids that differ in the other fifteen sequences are noted within each of the aligned sequences and are designated as single letter amino acid codons. Framework (FR) and complementarity determining (CDR) regions are marked above the sequences, and amino acid positions are marked. Designation of each mAb sequence is listed to the left of each sequence.

FIGS. 6A-6B show h6E6-116 and h6E6-070 humanized anti-human ROR2 mAb specifically bind human ROR2. (FIG. 6A) Binding of the h6E6-116 and h6E6-070 mAb to human ROR2 was assessed by flow cytometric staining and analysis of several human cell lines known to express ROR2. Cells were stained on ice for 20 minutes with 5 μg/ml of h6E6-116 or h6E6-070 anti-human ROR2-Alexa647 conjugated mAb (shaded histograms) or equal amounts of isotype matched control mAb (open histograms), washed and analyzed. Histograms depict the relative fluorescence intensity (x axis) of viable cells as determined by light scatter characteristics. (FIG. 6B) Specificity was validated by the absence of binding to HCT116 colorectal cancer cells in which ROR2 expression was eliminated using CRISPR-cas9, compared to parental cell lines that express ROR2, or MEC1 leukemia cells transduced to express human ROR2.

FIG. 7 shows h6E6-116 humanized anti-human ROR2 mAb do not bind to lymphocytes (Lymphs) or peripheral blood mononuclear cells (PBMC) isolated from healthy donors. Blood cells were isolated by Ficoll density centrifugation from whole blood obtained with consent from healthy donors. Cells were stained with 5 μg/ml of h6E6-116 anti-human ROR2-Alexa647 conjugated mAb (shaded histograms) or equal amounts of isotype matched control mAb (open histograms) on ice for 20 minutes. washed and analyzed by flow cytometry. Histograms depict staining of live viable mononuclear cells (PMBC) or lymphocytes (Lymphs) as determined by light scatter characteristics. Staining of K562 cells was performed as a positive control.

FIG. 8 shows h6E6-116 humanized anti-human ROR2 chimeric antigen receptors (CAR) bind specifically to recombinant human ROR2 protein. Jurkat cells were transfected with a h6E6-116 anti-human ROR2 chimeric antigen receptor construct or an anti-human ROR1 CAR construct. All cells were assessed after 48 hours by flow cytometry for binding of recombinant human ROR2-Ig protein comprised of the ROR2 extracellular domain and the CH2-CH3 constant region domains of human IgG1. Cells were stained on ice for 20 minutes with 1 μg/ml of recombinant ROR2-Ig (upper panel) or 1 μg/ml of control ROR1-Ig (lower panel). washed, stained for an additional 20 minutes on ice with phycoerythrin (PE)-conjugated anti-human IgG1 antibody, washed and analyzed by flow cytometry. Shaded histograms depict the relative fluorescence intensity (x axis) of viable cells, as determined by light scatter characteristics, stained with either ROR2-Ig or ROR1-Ig compared to cells stained with the anti-human IgG1-PE antibody only (open histograms). Jurkat cells transfected with a h6E6-116 a CAR construct binds ROR2-Ig (upper panels), but not ROR1-Ig, which is comprised of the same human IgG domains fused to the extracellular region of human ROR1. Conversely, ROR1 CAR transfected cells bind only to ROR1-Ig.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents. cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid. e.g. polynucleotides contemplated herein include any types of RNA. e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including. e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the intemucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., ROR2) in other proteins with different numbering systems. For example, by performing a simple sequence alignment with a protein (e.g., ROR2) the identity and location of residues corresponding to specific positions of the protein are identified in other protein sequences aligning to the protein. For example, a selected residue in a selected protein corresponds to aspartic acid at position 354 when the selected residue occupies the same essential spatial or other structural relationship as an aspartic acid at position 354. In some embodiments, where a selected protein is aligned for maximum homology with a protein, the position in the aligned selected protein aligning with aspartic acid 354 is the to correspond to aspartic acid 354. Instead of a primary sequence alignment, a three dimensional structural alignment can also be used, e.g., where the structure of the selected protein is aligned for maximum correspondence with the aspartic acid at position 354, and the overall structures compared. In this case, an amino acid that occupies the same essential position as aspartic acid 354 in the structural model is the amino acid to correspond to the aspartic acid 354 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations.” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K):
5) Isoleucine (I). Leucine (L), Methionine (M), Valine (V):
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues: always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments: or the end of either sequence is reached. The BLAST algorithm parameters W. T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Antibodies are large, complex molecules (molecular weight of ~150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”. “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The term “antibody” is used according to its commonly known meaning in the art. Antibodies exist. e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see. e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar. Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816.567; 5,530.101; 5,859,205; 5,585,089: 5,693,761; 5.693,762; 5,777,085; 6,180,370; 6,210,671; and 6,329,511; WO 87/02671: EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi. Adv. Immunol., 44:65-92 (1988), Verhoeven et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.: or (b) the variable region, or a portion thereof, is altered, replaced or exchanged with a variable region having a different or altered antigen specificity. The preferred antibodies of, and for use according to the invention include humanized and/or chimeric monoclonal antibodies.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, the solid-phase enzyme-linked immunosorbent assay (ELISA) is routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications. Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to to an antibody.

The term “ROR2 protein” or “ROR2” as used herein includes any of the recombinant or naturally-occurring forms of Receptor tyrosine kinase-like orphan receptor 2, also known as Tyrosine-protein kinase transmembrane receptor ROR2. Neurotrophic tyrosine kinase receptor-related 2, or variants or homologs thereof that maintain ROR2 activity (e.g. within at least 50%, 80%, 90%, 95%, 96%, 97%, 98%, 99% or 100% activity compared to ROR2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ROR2 protein. In embodiments, the ROR2 protein is substantially identical to the protein identified by SEQ ID NO:18. In embodiments, the ROR2 protein is substantially identical to the protein identified by the UniProt reference number Q01974 or a variant or homolog having substantial identity thereto. In embodiments, the ROR2 protein is substantially identical to the protein identified by the UniProt reference number A1L4F5 or a variant or homolog having substantial identity thereto. In embodiments, the ROR2 protein is substantially identical to the protein identified by the UniProt reference number Q8C3W2 or a variant or homolog having substantial identity thereto.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art.

For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “label” or a “detectable moiety” is a composition detectable by spectroscopic, photochemical, biochemical, immunochemical, chemical, or other physical means. For example, useful labels include phosphorus-32 (³²P), fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for magnetic resonance imaging (MRI), when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as radium-223 (²²³Ra) for radioimmunetherapy (RAIT) may be used. In certain embodiments, chelating moieties may be used to attach a positron emission tomography (PET) imaging agent, such as an Al-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein (e.g. ROR2 protein). Similarly an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

As defined herein, the term “inhibition”, “inhibit”, “inhibiting” and the like in reference to a protein-inhibitor interaction means negatively affecting (e.g. decreasing) the activity or function of the protein (e.g. ROR2 protein) relative to the activity or function of the protein in the absence of the inhibitor. In embodiments inhibition means negatively affecting (e.g. decreasing) the concentration or levels of ROR2 relative to the concentration or level of the protein in the absence of the inhibitor. In embodiments inhibition refers to reduction of a disease or symptoms of disease. In embodiments, inhibition refers to a reduction in the activity of ROR2. Thus, inhibition includes, at least in part, partially or totally blocking stimulation, decreasing, preventing, or delaying activation, or inactivating, desensitizing, or down-regulating signal transduction or enzymatic activity or the amount of ROR2. In embodiments, inhibition refers to a reduction of activity of ROR2 resulting from a direct interaction (e.g. an inhibitor binds to ROR2). In embodiments, inhibition refers to a reduction of activity of ROR2 from an indirect interaction (e.g. an inhibitor binds to a protein that activates ROR2, thereby preventing target protein activation).

Thus, the terms “inhibitor,” “repressor” or “antagonist” or “downregulator” interchangeably refer to a substance capable of detectably decreasing the expression or activity of a given gene or protein (e.g. ROR2 protein). The antagonist can decrease ROR2 expression or activity 10%, 20%, 30%, 40%, 50%, 60%, 70%, 80%, 90% or more in comparison to a control in the absence of the antagonist. In certain instances, ROR2 expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. The cancer may refer to a leukemia, lymphoma, or solid tumor malignancy. Solid tumor malignancies include malignant tumors that may be devoid of fluids or cysts. The solid tumor malignancy may be any one of a variety of different cancers (e.g., breast cancer, ovarian cancer, pancreatic cancer, cervical cancer, gastric cancer, renal cancer, head and neck cancer, bone cancer, skin cancer or prostate cancer). In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), acute lymphocytic (ALL), and chronic myelogenous leukemia (CML), multiple myeloma, or hairy-cell leukemia).

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, kidney cancer, leukemia, lung cancer, melanoma, ovarian cancer, prostate cancer, pancreatic cancer, brain cancer, liver cancer, gastric cancer or a sarcoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute myeloid leukemia, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia. Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifomi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma. Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations. e.g., in the breast.

The term “associated” or “associated with” in the context of a substance or substance activity or function associated with a disease (e.g. a protein associated disease, a cancer associated with ROR2 activity, ROR2 associated cancer, ROR2 associated disease (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease. For example, a cancer associated with ROR2 activity or function or a ROR2 associated disease (e.g., cancer, inflammatory disease, autoimmune disease, or infectious disease), may be treated with a ROR2 modulator or ROR2 inhibitor, in the instance where increased ROR2 activity or function (e.g. signaling pathway activity) causes the disease (e.g., cancer, inflammatory disease, autoimmune disease, or infectious disease). For example, an inflammatory disease associated with ROR2 activity or function or an ROR2 associated inflammatory disease, may be treated with an ROR2 modulator or ROR2 inhibitor, in the instance where increased ROR2 activity or function (e.g. signaling pathway activity) causes the disease.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as cancer (e.g., leukemia). In embodiments, the therpaeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

An “anticancer agent” as used herein refers to a molecule (e.g. compound, peptide, protein, nucleic acid, 0103) used to treat cancer through destruction or inhibition of cancer cells or tissues. Anticancer agents may be selective for certain cancers or certain tissues. In embodiments, anticancer agents herein may include epigenetic inhibitors and multi-kinase inhibit “Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.R™.), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil: abiraterone; aclarubicin; acylfulvene; adecypenol: adozelesin: aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston: antisense oligonucleotides; aphidicolin glycinate; apoptosis gene modulators; apoptosis regulators; apurinic acid; ara-CDP-DL-PTBA: arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives: beta-alethine; betaclamycin B: betulinic acid; bFGF inhibitor: bicalutamide; bisantrene; bisaziridinylspermine: bisnafide; bistratene A; bizelesin; breflate; bropirimine; budotitane; buthionine sulfoximine: calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol. 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin IL, or rlL,sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules. (e.g. Taxol.™ (i.e. paclitaxel), Taxotere.™, compounds comprising the taxane skeleton, Erbulozole (i.e. R-55104), Dolastatin 10 (i.e. DLS-10 and NSC-376128), Mivobulin isethionate (i.e. as CI-980). Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356). Epothilones (e.g. Epothilone A, Epothilone B. Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone. Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis). SDZ-268970 (Lilly/Novartis). AM-97 (Armad/Kyowa Hakko). AM-132 (Armad), AM-138 (Armad/KyowaHakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl. and RPR-258062A), Vitilevuamide, Tubulysin A, Canadensol, Centaureidin (i.e. NSC-106969), T-138067 (Tularik, i.e. T-67. TL-138067 and TI-138067), COBRA-1 (Parker Hughes Institute, i.e. DDE-261 and WHI-261), H1O (Kansas State University), H16 (Kansas State University), Oncocidin Al (i.e. BTO-956 and DIME), DDE-313 (Parker Hughes Institute), Fijianolide B, Laulimalide, SPA-2 (Parker Hughes Institute), SPA-1 (Parker Hughes Institute. i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine). A-204197 (Abbott), T-607 (Tuiarik. i.e. T-900607). RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A. A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to Indium-111 (¹¹In), Yitrium-90 (⁹⁰Y), or Iodine-131 (¹³¹1), etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib. EGFR inhibitors, epidermal growth factor receptor (EGFR)-targeted therapy or therapeutic (e.g. gefitinib (Iressa™), erlotinib (Tarceva™) cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethvl erlotinib, AZD8931, AEE788, pelitinib/EKB-569, CUDC-101, WZ8040, WZ4002. WZ3146, AG-490. XL647, PD153035, BMS-599626), sorafenib, imatinib, sunitinib, dasatinib, or the like.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%. 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization. e.g., for inhalants, in a tablet or liquid. e.g., for oral delivery, or a saline solution. e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes. e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdemeral administration, by a topical route, formulated as applicator sticks, solutions, suspensions, emulsions, gels, creams, ointments, pastes, jellies, paints, powders, or aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn. Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### ANTI-ROR2 Antibodies

Provided herein are, inter alia, antibodies (e.g., humanized antibodies, monoclonal antibodies), antibody fragments (e.g., scFvs) and antibody compositions (e.g., chimeric antigen receptors, bispecific antibodies), which bind human tyrosine kinase-like orphan receptor 2 (ROR2) with high efficiency and specificity. The antibodies and antibody compositions provided herein include novel light and heavy chain domain CDRs and framework regions and have been identified to bind extracellular domains of human ROR2. For example, the antibodies provided herein including embodiments thereof, may bind the Kringle or the Ig-like domain of ROR2 with high affinity and specificity. Further, Applicants have characterized the amino acid residues in the ROR2 extracellular domains, which are important for binding of antibodies as described herein including embodiments thereof. Antibodies specifically binding the epitope described herein including embodiments thereof, are useful for binding human ROR2 with high effectivity and affinity and inhibiting ROR2 signaling in cells expressing ROR2. The antibodies provided herein including embodiments thereof, may be used for diagnostic and therapeutic purposes in cancer and other ROR2-related diseases. The variable light chain and the variable heavy chain domains provided herein may, inter alia, form part of an anti-ROR2 chimeric antigen receptor or an anti-ROR2 bispecific antibody. Furthermore, due to their internalization properties, some of the anti-ROR2 antibodies provided herein may be attached to therapeutic moieties and used as antibody-drug conjugates (ADC), or they may be attached to a detectable moiety and used for diagnostic purposes. The antibodies provided herein including embodiments thereof, have an ability to inhibit migration of ROR2 expressing metastatic cells and therefore are capable of mitigating the risk of metastasis in patients with ROR2-expressing cancer cells

As described above, a “light chain variable (VL) domain” as provided herein refers to the variable region of the light chain of an antibody, an antibody variant or fragment thereof. Likewise, the “heavy chain variable (VH) domain” as provided herein refers to the variable region of the heavy chain of an antibody, an antibody variant or fragment thereof. The light chain variable domain and the heavy chain variable domain together form the paratope, which binds an antigen (epitope). The paratope or antigen-binding site is formed at the N-terminus of an antibody, an antibody variant or fragment thereof. In embodiments, the light chain variable (VL) domain includes CDR L1, CDR L2, CDR L3 and FR L1, FR L2, FR L3 and FR L4 (framework regions) of an antibody light chain. In embodiments, the heavy chain variable (VH) domain includes CDR H1, CDR H2, CDR H3 and FR H1, FR H2, FR H3 and FR H4 (framework regions) of an antibody heavy chain. In embodiments, the light chain variable (VL) domain and a light chain constant (CL) domain form part of an antibody light chain. In embodiments, the heavy chain variable (VH) domain and a heavy chain constant (CH1) domain form part of an antibody heavy chain. In embodiments, the heavy chain variable (VH) domain and one or more heavy chain constant (CH1, CH2, or CH3) domains form part of an antibody heavy chain. Thus, in embodiments, the light chain variable (VL) domain forms part of an antibody. In embodiments, the heavy chain variable (VH) domain forms part of an antibody. In embodiments, the light chain variable (VL) domain forms part of a therapeutic antibody. In embodiments, the heavy chain variable (VH) domain forms part of a therapeutic antibody. In embodiments, the light chain variable (VL) domain forms part of a human antibody. In embodiments, the heavy chain variable (VH) domain forms part of a human antibody. In embodiments, the light chain variable (VL) domain forms part of a humanized antibody. In embodiments, the heavy chain variable (VH) domain forms part of a humanized antibody. In embodiments, the light chain variable (VL) domain forms part of a chimeric antibody. In embodiments, the heavy chain variable (VH) domain forms part of a chimeric antibody. In embodiments, the light chain variable (VL) domain forms part of an antibody fragment. In embodiments, the heavy chain variable (VH) domain forms part of an antibody fragment. In embodiments, the light chain variable (VL) domain forms part of an antibody variant. In embodiments, the heavy chain variable (VH) domain forms part of an antibody variant. In embodiments, the light chain variable (VL) domain forms part of a Fab. In embodiments, the heavy chain variable (VH) domain forms part of a Fab. In embodiments, the light chain variable (VL) domain forms part of a scFv. In embodiments, the heavy chain variable (VH) domain forms part of a scFv

In embodiments, the antibody does not bind an arginine at a position corresponding to position 349 of SEQ ID NO:36. In embodiments, the antibody does not bind a glutamic acid at a position corresponding to position 354 of SEQ ID NO:36. In embodiments, the antibody does not bind an arginine at a position corresponding to position 349 of SEQ ID NO:36. In embodiments, the antibody does not bind a glutamic acid at a position corresponding to position 354 of SEQ ID NO:36.

In embodiments, the antibody binds the Kringle domain of human ROR2.

In embodiments, the antibody binds a cancer cell. In embodiments, the cancer cell is a breast cancer cell, ovarian cancer cell, pancreatic cancer cell, cervical cancer cell, gastric cancer cell, renal cancer cell, head and neck cancer cell, bone cancer cell, skin cancer cell or prostate cancer cell.

In embodiments, the antibody is attached to a therapeutic agent. In embodiments, the antibody is attached to a diagnostic agent. In embodiments, the diagnostic agent is a detectable moiety.

In embodiments, the antibody is capable of binding a ROR2 protein. In embodiments, the ROR2 protein is a human ROR2 protein. In embodiments, the antibody binds a ROR2 protein. In embodiments, the antibody is capable of binding an extracellular domain of the ROR2 protein. In embodiments, the antibody binds an extracellular domain of the ROR2 protein. In embodiments, the extracellular domain is an Ig-like domain.

The antibodies provided herein including embodiments thereof may be humanized antibodies. Thus, in embodiments, the antibody is a humanized antibody. In embodiments, the antibody is a chimeric antibody. In embodiments, antibody is a Fab′ fragment. In embodiments, the antibody is an IgG. In embodiments, the antibody is an IgG. The anti-ROR2 antibody provided herein may be an IgG1, IgG2, IgG3 or IgG4. In embodiments, the antibody is an IgG1. In embodiments, the antibody is an IgG2. In embodiments, the antibody is an IgG2a. In embodiments, the antibody is an IgG3. In embodiments, the antibody is an IgG4.

In embodiments, the antibody does not bind a mouse ROR2 protein. In embodiments, the antibody does not bind a mouse ROR2 protein identified by the UniProt reference number Q9Z138. In embodiments, the antibody provided herein does not bind a protein comprising the amino acid sequence of SEQ ID NO:36.

In an aspect, the ROR2 protein bound by the antibodies provided herein including embodiments thereof is expressed by a cell (e.g., cancer cell). Thus, in embodiments, the ROR2 protein is expressed on a cell. In embodiments, the cell is a cancer cell. In embodiments, the cancer cell is cancer is a breast cancer cell, ovarian cancer cell, pancreatic cancer cell, cervical cancer cell, gastric cancer cell, renal cancer cell, head and neck cancer cell, bone cancer cell, skin cancer cell or prostate cancer cell. In embodiments, the cancer cell is a breast cancer cell. In embodiments, the cancer cell is an ovarian cancer cell. In embodiments, the cancer cell is a pancreatic cancer cell. In embodiments, the cancer cell is cancer is a cervical cancer cell. In embodiments, the cancer cell is cancer is a gastric cancer cell. In embodiments, the cancer cell is cancer is a renal cancer cell. In embodiments, the cancer cell is cancer is a head and neck cancer cell. In embodiments, the cancer cell is a cancer is bone cancer cell. In embodiments, the cancer cell is cancer is a skin cancer cell. In embodiments, the cancer cell is cancer is a prostate cancer cell.

In embodiments, the antibody provided herein does not bind a ROR2-negative cell. A “ROR2-negative cell” as provided herein is a cell that does not express a detectable amount of a ROR2 protein relative to a standard control. In embodiments, the expression level of a ROR2-negative cell is undetectable using methods conventionally known in the art to a detect protein expression in a cell (e.g., immunofluorescent detection, protein biochemistry, RNA expression level). In embodiments, the expression level of a ROR2-negative cell is 1000, 500, 100, 50, 25, 20, 10, 5, or 1.5 times lower than the expression level of a standard control (e.g., a cell expressing detectable levels of ROR2 using conventional methods). No limiting examples of ROR2-negative cells include a peripheral blood mononuclear cell (PBMC) from a healthy subject.

In an aspect is provided, a method of identifying an anti-ROR2 antibody. The method includes: (i) contacting an antibody with a first ROR2 polypeptide including a histidine at a position corresponding to position 349 of SEQ ID NO:34; (ii) detecting the antibody binding to the first ROR2 polypeptide; (iii) contacting the antibody with a second ROR2 polypeptide not including a histidine at a position corresponding to position 349 of SEQ ID NO:34; and (iv) detecting the antibody not binding to the second ROR2 polypeptide, thereby identifying an anti-ROR2 antibody.

In an aspect is provided, a method of identifying an anti-ROR2 antibody. The method includes: (i) contacting an antibody with a first ROR2 polypeptide including an aspartic acid at a position corresponding to position 354 of SEQ ID NO:34; (ii) detecting the antibody binding to the first ROR2 polypeptide; (iii) contacting the antibody with a second ROR2 polypeptide not including an aspartic acid at a position corresponding to position 354 of SEQ ID NO:34; and (iv) detecting the antibody not binding to the second ROR2 polypeptide, thereby identifying an anti-ROR2 antibody.

In an aspect is provided, a method of identifying an anti-ROR2 antibody. The method includes: (i) contacting an antibody with a first ROR2 polypeptide including a methionine at a position corresponding to position 386 of SEQ ID NO:34: (ii) detecting the antibody binding to the first ROR2 polypeptide; (iii) contacting the antibody with a second ROR2 polypeptide not including a methionine at a position corresponding to position 386 of SEQ ID NO:34; and (iv) detecting the antibody not binding to the second ROR2 polypeptide, thereby identifying an anti-ROR2 antibody.

In an aspect is provided, an anti-tyrosine kinase-like orphan receptor 2 (ROR2) antibody including a heavy chain variable domain and a light chain variable domain, wherein the heavy chain variable domain includes the sequence of SEQ ID NO: 1 and wherein the light chain variable domain includes the sequence of SEQ ID NO: 2; wherein the heavy chain variable domain includes the sequence of SEQ ID NO: 3 and wherein the light chain variable domain includes the sequence of SEQ ID NO: 4: wherein the heavy chain variable domain includes the sequence of SEQ ID NO: 5 and wherein the light chain variable domain includes the sequence of SEQ ID NO: 6; wherein the heavy chain variable domain includes the sequence of SEQ ID NO: 7 and wherein the light chain variable domain includes the sequence of SEQ ID NO: 8; wherein the heavy chain variable domain includes the sequence of SEQ ID NO: 9 and wherein the light chain variable domain includes the sequence of SEQ ID NO: 10: wherein the heavy chain variable domain includes the sequence of SEQ ID NO: 11 and wherein the light chain variable domain includes the sequence of SEQ ID NO: 12; wherein the heavy chain variable domain includes the sequence of SEQ ID NO: 13 and wherein the light chain variable domain includes the sequence of SEQ ID NO: 14: wherein the heavy chain variable domain includes the sequence of SEQ ID NO: 15 and wherein the light chain variable domain includes the sequence of SEQ ID NO: 16; wherein the heavy chain variable domain includes the sequence of SEQ ID NO: 17 and wherein the light chain variable domain includes the sequence of SEQ ID NO: 18; wherein the heavy chain variable domain includes the sequence of SEQ ID NO: 19 and wherein the light chain variable domain includes the sequence of SEQ ID NO: 20: wherein the heavy chain variable domain includes the sequence of SEQ ID NO: 21 and wherein the light chain variable domain includes the sequence of SEQ ID NO: 22; wherein the heavy chain variable domain includes the sequence of SEQ ID NO: 23 and wherein the light chain variable domain includes the sequence of SEQ ID NO: 24: wherein the heavy chain variable domain includes the sequence of SEQ ID NO: 25 and wherein the light chain variable domain includes the sequence of SEQ ID NO: 26; wherein the heavy chain variable domain includes the sequence of SEQ ID NO: 27 and wherein the light chain variable domain includes the sequence of SEQ ID NO: 28; wherein the heavy chain variable domain includes the sequence of SEQ ID NO: 29 and wherein the light chain variable domain includes the sequence of SEQ ID NO: 30; or wherein the heavy chain variable domain includes the sequence of SEQ ID NO: 31 and wherein the light chain variable domain includes the sequence of SEQ ID NO: 32.

In an aspect is provided an anti-ROR2 antibody capable of binding the same epitope as an antibody provided herein including embodiments thereof.

In an aspect is provided a method of treating cancer in a subject in need thereof, the method including administering to a subject a therapeutically effective amount of an antibody as provided herein including embodiments thereof.

In an aspect is provided a method of inhibiting metastasis of a ROR2-expressing cancer in a subject in need thereof, the method including administering to a subject a therapeutically effective amount of an antibody as provided herein including embodiments thereof.

In an aspect is provided anti-tyrosine kinase-like orphan receptor 2 (ROR2) antibody including: (i) a CDR H1 including an amino acid sequence of formula: G-F-T-F-S-X1-Y-G-X2-X3 (I), wherein X1 is A, D, E, F, G, H, K, N, P, Q, R, S, T, or V, X2 is A, H, I, L, M, Q, S, T, or V, X3 is S or N, and wherein if X2 is M and X3 is S X1 is not N; (ii) a CDR H2 including an amino acid sequence of formula: X4-I-S-S-G-G-G-Y-T-X5-Y-X6 (II), wherein X4 is T or S, X5 is H or Y. X6 is V or A; (iii) a CDR H3 including an amino acid sequence of formula: A-R-H-P-R-D-F-S-Y-A-X7-D-Y (III); wherein X7 is A, F, H, I, K, L, M, N, Q, S, T, or V; (iv) a CDR L1 including an amino acid sequence of formula: Q-D-V-G-H-Y (IV); (v) a CDR L2 including an amino acid sequence of formula: W-A-S (V); and (vi) a CDR L3 including the sequence of SEQ ID NO:53.

In embodiments, the antibody further includes a FR H3 including an amino acid sequence of formula: X11-S-V-K-G (VI), wherein X11 is A, D, E, G, H, P, Q, or T.

In another aspect is provided an anti-tyrosine kinase-like orphan receptor 2 (ROR2) antibody including: (i) a CDR H1 including an amino acid sequence of formula: G-F-T-F-S-X1-Y-G-X2-X3 (I), wherein X1 is A, D, E, F, G, H, I, K, L, N, P, Q, R, S, T, V, W, or Y, X2 is A, E, F, G, H, I, K, L, M, P, Q, R, S, T, V, W, or Y. X3 is S or N and wherein if X2 is M and X3 is S X1 is not N; (ii) a CDR H2 including an amino acid sequence of formula: X4-I-S-S-G-G-G-Y-T-X5-Y-X6 (II), wherein X4 is T or S, X5 is H or Y, X6 is V or A, and wherein if X5 is H and X6 is V X4 is not T; (iii) a CDR H3 including an amino acid sequence of formula: D-F-S-Y-A-X7-D-Y-W-G (III); wherein X7 is A, D, E, F, G, H, I, K, L, M, P, Q, R, S, T, V, W, or Y; (iv) a CDR L1 including an amino acid sequence of formula: X8-A-S-Q-D-V-G-H-Y-X9-A (IV) wherein X8 is K or R, X9 is V or L, and wherein if X8 is K X9 is not V; (v) a CDR L2 including an amino acid sequence of formula: W-A-S-T-R-X10-T (V) wherein X10 is H or A, and wherein if X8 is K and X9 is V X10 is not H; and (vi) a CDR L3 including the sequence of SEQ ID NO:53.

In another aspect is provided an anti-tyrosine kinase-like orphan receptor 2 (ROR2) antibody including: (i) a CDR H1 including an amino acid sequence of formula: G-F-T-F-S-X1-Y-G-X2-X3 (I), wherein X1 is A, D, E, F, G, H, K, N, P, Q, R, S, T, or V, X2 is A, , , H, I, , L, M, , Q, , S, T, or V, X3 is S or N, and wherein if X2 is M and X3 is S X1 is not N; (ii) a CDR H2 including an amino acid sequence of formula: X4-I-S-S-G-G-G-Y-T-X5-Y-X6 (II), wherein X4 is T or S, X5 is H or Y, X6 is V or A, and wherein if X5 is H and X6 is V X4 is not T; (iii) a CDR H3 including an amino acid sequence of formula: A-R-H-P-R-D-F-S-Y-A-X7-D-Y (III); wherein X7 is A, F, H, I, K, L, M, N, P, Q, S, T, or V; (iv) a CDR L1 including an amino acid sequence of formula: Q-D-V-G-H-Y: (v) a CDR L2 including an amino acid sequence of formula: W-A-S; and (vi) a CDR L3 including the sequence of SEQ ID NO:53.

In embodiments, X1 is A. In embodiments, X1 is D, In embodiments, X1 is E. In embodiments, X1 is F. In embodiments. X1 is G. In embodiments, X1 is H, In embodiments, X1 is I, In embodiments, X1 is K. In embodiments, X1 is L, In embodiments. X1 is N. In embodiments, X1 is P, In embodiments, X1 is Q. In embodiments, X1 is R. In embodiments, X1 is S. In embodiments, X1 is T, In embodiments, X1 is V. In embodiments, X1 is W. In embodiments, X1 is Y.

In embodiments, X2 is A. In embodiments, X2 is E. In embodiments, X2 is F. In embodiments, X2 is G. In embodiments, X2 is H, In embodiments, X2 is I, In embodiments, X2 is K. In embodiments, X2 is L, In embodiments, X2 is M. In embodiments, X2 is P, In embodiments, X2 is Q. In embodiments, X2 is R. In embodiments, X2 is S. In embodiments, X2 is T, In embodiments, X2 is V. In embodiments, X2 is W. In embodiments, X2 is Y.

In embodiments, X3 is S. In embodiments, X3 is N.

In embodiments, if X2 is M and X3 is S, X1 is not N. In embodiments, if X2 is M, X1 is not N. In embodiment, if X3 is S, X1 is not N. In embodiments, if X2 is M and X3 is S X1 is A, D, E, F, G, H, I, K, L, P, Q, R, S, T, V, W, or Y. In embodiments, X2 is M and X1 is A, D, E, F, G, H, I, K, L, P, Q, R, S, T, V, W, or Y. In embodiments, X3 is S and X1 is A, D, E, F, G, H, I, K, L, P, Q, R, S, T, V, W, or Y. In embodiments X2 is M and X3 is S. In embodiments, if X2 is M and X3 is S, X1 is A. In embodiments, if X2 is M and X3 is S, X1 is D, In embodiments if X2 is M and X3 is S, X1 is E. In embodiments, if X2 is M and X3 is S. X1 is F. In embodiments, if X2 is M and X3 is S, X1 is G. In embodiments, if X2 is M and X3 is S. X1 is H, In embodiments, if X2 is M and X3 is S, X1 is I, In embodiments, if X2 is M and X3 is S, X1 is K. In embodiments, if X2 is M and X3 is S, X1 is L, In embodiments, if X2 is M and X3 is S, X1 is P, In embodiments, if X2 is M and X3 is S, X1 is Q. In embodiments, if X2 is M and X3 is S, X1 is R. In embodiments, if X2 is M and X3 is S, X1 is S. In embodiments, if X2 is M and X3 is S. X1 is T, In embodiments, if X2 is M and X3 is S. X1 is V. In embodiments, if X2 is M and X3 is S, X1 is W. In embodiments, if X2 is M and X3 is S, X1 is Y. In embodiments, X2 is M, X3 is S and X1 is A. In embodiments, X2 is M, X3 is S and X1 is D, In embodiments, X2 is M, X3 is S and X1 is E. In embodiments, X2 is M, X3 is S and X1 is F. In embodiments, X2 is M, X3 is S and X1 is G. In embodiments. X2 is M. X3 is S and X1 is H, In embodiments. X2 is M, X3 is S and X1 is I, In embodiments, X2 is M, X3 is S and X1 is K. In embodiments, X2 is M, X3 is S and X1 is L, In embodiments, X2 is M, X3 is S and X1 is P, In embodiments, X2 is M, X3 is S and X1 is Q. In embodiments, X2 is M, X3 is S and X1 is R. In embodiments, X2 is M, X3 is S and X1 is S. In embodiments, X2 is M, X3 is S and X1 is T, In embodiments, X2 is M, X3 is S and X1 is V. In embodiments, X2 is M, X3 is S and X1 is W. In embodiments, X2 is M, X3 is S and X1 is Y.

In embodiments, X1 is A, D, E, F, G, H, I, K, L, N, P, Q, R, S, T, V, W, or Y and X2 is A. In embodiments, X1 is A, D, E, F, G, H, I, K, L, N, P, Q, R, S, T, V, W, or Y and X2 is E. In embodiments, X1 is A, D, E, F, G, H, I, K, L, N, P, Q, R, S, T, V, W, or Y and X2 is F. In embodiments, X1 is A, D, E, F, G, H, I, K, L, N, P, Q, R, S, T, V, W, or Y and X2 is G. In embodiments, X1 is A, D, E, F, G, H, I, K, L, N, P, Q, R, S, T, V, W, or Y and X2 is H, In embodiments, X1 is A, D, E, F, G, H, I, K, L, N, P, Q, R, S, T, V, W, or Y and X2 is I, In embodiments, X1 is A, D, E, F, G, H, I, K, L, N, P, Q, R, S, T, V, W, or Y and X2 is K. In embodiments, X1 is A, D, E, F, G, H, I, K, L, N, P, Q, R, S, T, V, W, or Y and X2 is L, In embodiments, X1 is A, D, E, F, G, H, I, K, L, N, P, Q, R, S, T, V, W, or Y and X2 is M. In embodiments, X1 is A, D, E, F, G, H, I, K, L, N, P, Q, R, S, T, V, W, or Y and X2 is P, In embodiments, X1 is A, D, E, F, G, H, I, K, L, N, P, Q, R, S, T, V, W, or Y and X2 is Q. In embodiments, X1 is A, D, E, F, G, H, I, K, L, N, P, Q, R, S, T, V, W, or Y and X2 is R. In embodiments, X1 is A, D, E, F, G, H, I, K, L, N, P, Q, R, S, T, V, W, or Y and X2 is S. In embodiments, X1 is A, D, E, F, G, H, I, K, L, N, P, Q, R, S, T, V, W, or Y and X2 is T, In embodiments, X1 is A, D, E, F, G, H, I, K, L, N, P, Q, R, S, T, V, W, or Y and X2 is V. In embodiments, X1 is A, D, E, F, G, H, I, K, L, N, P, Q, R, S, T, V, W, or Y and X2 is W. In embodiments, X1 is A, D, E, F, G, H, I, K, L, N, P, Q, R, S, T, V, W, or Y and X2 is Y. In one further embodiment, X3 is N. In another further embodiment, X3 is S.

In embodiments, X4 is T, In embodiments, X4 is S. In embodiments, X5 is H, In embodiments, X5 is Y. In embodiments, X6 is V. In embodiments, X6 is A. In embodiments, if X5 is H and X6 is V. X4 is not T, In embodiments, X5 is H, X6 is V, and X4 is S. In embodiments, X5 is Y. X6 is V, and X4 is T, In embodiments, X5 is Y, X6 is V. and X4 is S. In embodiments, X5 is Y, X6 is A, and X4 is T, In embodiments, X5 is Y, X6 is A, and X4 is S. In embodiments, X5 is H, X6 is A, and X4 is T, In embodiments, X5 is H, X6 is A, and X4 is S. In embodiments, if X5 is H, X4 is not T, In embodiments, if X6 is V. X4 is not T, In embodiments, if X5 is H and X6 is V, X4 is S. In embodiments, X4 is T or S and X5 is H, In embodiments, X4 is T or S and X5 is Y. In one further embodiment, X6 is V. In another further embodiment, X6 is A.

In embodiments, X7 is A. In embodiments, X7 is D, In embodiments, X7 is E. In embodiments, X7 is F. In embodiments. X7 is G. In embodiments, X7 is H, In embodiments, X7 is I, In embodiments, X7 is K. In embodiments, X7 is L, In embodiments. X7 is M. In embodiments, X7 is P, In embodiments, X7 is Q. In embodiments, X7 is R. In embodiments, X7 is S. In embodiments, X7 is T, In embodiments, X7 is V. In embodiments, X7 is W. In embodiments, X7 is Y.

In embodiments, X8 is K. In embodiments, X8 is R. In embodiments, X9 is V. In embodiments, X9 is L, In embodiments, if X8 is K, X9 is not V. In embodiments, if X8 is K, X9 is L, In embodiments, X8 is K or R and X9 is L, In embodiments, X9 is V or L and X8 is R.

In embodiments, X8 is K and X9 is L, In embodiments, X8 is R and X9 is V. In embodiments, X8 is R and X9 is L, In embodiments, X8 is K and X9 is L.

In embodiments, X10 is H, In embodiments, X10 is A. In embodiments, if X8 is K and X9 is V, X10 is not H, In embodiments, if X8 is K and X9 is V, X10 is A. In embodiments, X8 is K, X9 is V. and X10 is A.

In embodiments, the antibody further includes a FR H3 including an amino acid sequence of formula: Y-T-H-Y-V-X11-S-V-K-G (VI), wherein X11 is A, D, E, G, H, P, Q, T, or V.

In embodiments, the antibody further includes a FR H3 including an amino acid sequence of formula: X11-S-V-K-G (VI), wherein X11 is A, D, E, G, H, P, Q, or T.

In embodiments, X11 is A. In embodiments, X11 is D, In embodiments, X11 is E. In embodiments, X11 is G. In embodiments, X11 is H, In embodiments, X11 is P, In embodiments, X11 is Q. In embodiments, X11 is T, In embodiments, X11 is V.

In embodiments, X1 is S, G, N, K, A, or H, In embodiments, X1 is S. In embodiments, X1 is G. In embodiments, X1 is N. In embodiments, X1 is K. In embodiments, X1 is A. In embodiments, X1 is H.

In embodiments, X2 is M, V, T, I or L, In embodiments, X2 is M. In embodiments, X2 is V. In embodiments, X2 is T, In embodiments, X2 is I, In embodiments, X2 is L.

In embodiments, X7 is M, L or Q. In embodiments, X7 is M. In embodiments, X7 is L, In embodiments, X7 is Q.

In embodiments, X11 is E, P, D, A, or T, In embodiments, X11 is E. In embodiments, X11 is P, In embodiments, X11 is D, In embodiments, X11 is A. In embodiments, X11 is T.

In embodiments, X1 is not N, X2 is not M. X7 is not M or X11 is not D, In embodiments, X1 is not N. X2 is not M. X7 is not M, and X11 is not D, In embodiments, X1 is not N. In embodiments, X2 is not M. In embodiments, X7 is not M. In embodiments, X11 is not D, In embodiments, X1 is not N, X2 is not M, X7 is not M and X11 is not D.

In embodiments, the antibody includes: a CDR H1 as set forth in SEQ ID NO:305, a CDR H2 as set forth in SEQ ID NO:306 and a CDR H3 as set forth in SEQ ID NO:307; and a CDR L1 as set forth in SEQ ID NO:308, a CDR L2 as set forth in SEQ ID NO:309, and a CDR L3 as set forth in SEQ ID NO:310. In embodiments, the antibody includes a heavy chain variable domain including the sequence of SEQ ID NO:203 and a light chain variable domain including the sequence of SEQ ID NO:204. In embodiments, the antibody includes a heavy chain variable domain having the sequence of SEQ ID NO:203 and a light chain variable domain having the sequence of SEQ ID NO:204. In embodiments, the antibody is referred to herein as clone h6E6-070.

In embodiments, the antibody includes: a CDR H1 as set forth in SEQ ID NO:311, a CDR H2 as set forth in SEQ ID NO:312 and a CDR H3 as set forth in SEQ ID NO:313; and a CDR L1 as set forth in SEQ ID NO:314, a CDR L2 as set forth in SEQ ID NO:315, and a CDR L3 as set forth in SEQ ID NO:316. In embodiments, the antibody includes a heavy chain variable domain including the sequence of SEQ ID NO:295 and a light chain variable domain including the sequence of SEQ ID NO:296. In embodiments, the antibody includes a heavy chain variable domain having the sequence of SEQ ID NO:295 and a light chain variable domain having the sequence of SEQ ID NO:296. In embodiments, the antibody is referred to herein as clone h6E6-116.

In embodiments, the antibody includes: a CDR H1 as set forth in SEQ ID NO:317, a CDR H2 as set forth in SEQ ID NO:318 and a CDR H3 as set forth in SEQ ID NO:319; and a CDR L1 as set forth in SEQ ID NO:320, a CDR L2 as set forth in SEQ ID NO:321, and a CDR L3 as set forth in SEQ ID NO:322. In embodiments, the antibody includes a heavy chain variable domain including the sequence of SEQ ID NO:77 and a light chain variable domain including the sequence of SEQ ID NO:78. In embodiments, the antibody includes a heavy chain variable domain having the sequence of SEQ ID NO:77 and a light chain variable domain having the sequence of SEQ ID NO:78. In embodiments, the antibody is referred to herein as clone h6E6-007.

In embodiments, the antibody includes: a CDR H1 as set forth in SEQ ID NO:323, a CDR H2 as set forth in SEQ ID NO:324 and a CDR H3 as set forth in SEQ ID NO:325; and a CDR L1 as set forth in SEQ ID NO:326, a CDR L2 as set forth in SEQ ID NO:327, and a CDR L3 as set forth in SEQ ID NO:328. In embodiments, the antibody includes a heavy chain variable domain including the sequence of SEQ ID NO:121 and a light chain variable domain including the sequence of SEQ ID NO:122. In embodiments, the antibody includes a heavy chain variable domain having the sequence of SEQ ID NO:121 and a light chain variable domain having the sequence of SEQ ID NO:122. In embodiments, the antibody is referred to herein as clone h6E6-029.

In embodiments, the antibody includes: a CDR H1 as set forth in SEQ ID NO:329, a CDR H2 as set forth in SEQ ID NO:330 and a CDR H3 as set forth in SEQ ID NO:31; and a CDR L1 as set forth in SEQ ID NO:332, a CDR L2 as set forth in SEQ ID NO:333, and a CDR L3 as set forth in SEQ ID NO:334. In embodiments, the antibody includes a heavy chain variable domain including the sequence of SEQ ID NO: 143 and a light chain variable domain including the sequence of SEQ ID NO:144. In embodiments, the antibody includes a heavy chain variable domain having the sequence of SEQ ID NO: 143 and a light chain variable domain having the sequence of SEQ ID NO: 144. In embodiments, the antibody is referred to herein as clone h6E6-040.

In embodiments, the antibody includes: a CDR H1 as set forth in SEQ ID NO:335, a CDR H2 as set forth in SEQ ID NO:336 and a CDR H3 as set forth in SEQ ID NO:337; and a CDR L1 as set forth in SEQ ID NO:338, a CDR L2 as set forth in SEQ ID NO:339, and a CDR L3 as set forth in SEQ ID NO:340. In embodiments, the antibody includes a heavy chain variable domain including the sequence of SEQ ID NO: 173 and a light chain variable domain including the sequence of SEQ ID NO:174. In embodiments, the antibody includes a heavy chain variable domain having the sequence of SEQ ID NO:173 and a light chain variable domain having the sequence of SEQ ID NO:174. In embodiments, the antibody is referred to herein as clone h6E6-055.

In embodiments, the antibody includes: a CDR H1 as set forth in SEQ ID NO:341, a CDR H2 as set forth in SEQ ID NO:342 and a CDR H3 as set forth in SEQ ID NO: 343; and a CDR L1 as set forth in SEQ ID NO:344, a CDR L2 as set forth in SEQ ID NO: 345, and a CDR L3 as set forth in SEQ ID NO:346. In embodiments, the antibody includes a heavy chain variable domain including the sequence of SEQ ID NO: 175 and a light chain variable domain including the sequence of SEQ ID NO:176. In embodiments, the antibody includes a heavy chain variable domain having the sequence of SEQ ID NO:175 and a light chain variable domain having the sequence of SEQ ID NO:176. In embodiments, the antibody is referred to herein as clone h6E6-056.

In embodiments, the antibody includes: a CDR H1 as set forth in SEQ ID NO:347, a CDR H2 as set forth in SEQ ID NO:348 and a CDR H3 as set forth in SEQ ID NO:349; and a CDR L1 as set forth in SEQ ID NO:350, a CDR L2 as set forth in SEQ ID NO:351, and a CDR L3 as set forth in SEQ ID NO:352. In embodiments, the antibody includes a heavy chain variable domain including the sequence of SEQ ID NO:177 and a light chain variable domain including the sequence of SEQ ID NO:178. In embodiments, the antibody includes a heavy chain variable domain having the sequence of SEQ ID NO: 177 and a light chain variable domain having the sequence of SEQ ID NO: 178. In embodiments, the antibody is referred to herein as clone h6E6-057.

In embodiments, the antibody includes: a CDR H1 as set forth in SEQ ID NO:353, a CDR H2 as set forth in SEQ ID NO:354 and a CDR H3 as set forth in SEQ ID NO:355; and a CDR L1 as set forth in SEQ ID NO:356, a CDR L2 as set forth in SEQ ID NO:357, and a CDR L3 as set forth in SEQ ID NO:358. In embodiments, the antibody includes a heavy chain variable domain including the sequence of SEQ ID NO: 179 and a light chain variable domain including the sequence of SEQ ID NO:180. In embodiments, the antibody includes a heavy chain variable domain having the sequence of SEQ ID NO: 179 and a light chain variable domain having the sequence of SEQ ID NO: 180. In embodiments, the antibody is referred to herein as clone h6E6-058.

In embodiments, the antibody includes: a CDR H1 as set forth in SEQ ID NO:359, a CDR H2 as set forth in SEQ ID NO:360 and a CDR H3 as set forth in SEQ ID NO:361; and a CDR L1 as set forth in SEQ ID NO:362, a CDR L2 as set forth in SEQ ID NO:363, and a CDR L3 as set forth in SEQ ID NO:364. In embodiments, the antibody includes a heavy chain variable domain including the sequence of SEQ ID NO: 189 and alight chain variable domain including the sequence of SEQ ID NO:190. In embodiments, the antibody includes a heavy chain variable domain having the sequence of SEQ ID NO:189 and a light chain variable domain having the sequence of SEQ ID NO:190. In embodiments, the antibody is referred to herein as clone h6E6-063.

In embodiments, the antibody includes: a CDR H1 as set forth in SEQ ID NO:365, a CDR H2 as set forth in SEQ ID NO:366 and a CDR H3 as set forth in SEQ ID NO:367; and a CDR L1 as set forth in SEQ ID NO:368, a CDR L2 as set forth in SEQ ID NO:369, and a CDR L3 as set forth in SEQ ID NO:370. In embodiments, the antibody includes a heavy chain variable domain including the sequence of SEQ ID NO: 199 and a light chain variable domain including the sequence of SEQ ID NO:200. In embodiments, the antibody includes a heavy chain variable domain having the sequence of SEQ ID NO:199 and a light chain variable domain having the sequence of SEQ ID NO:200. In embodiments, the antibody is referred to herein as clone h6E6-068.

In embodiments, antibody includes: a CDR H1 as set forth in SEQ ID NO:371, a CDR H2 as set forth in SEQ ID NO:372 and a CDR H3 as set forth in SEQ ID NO:373; and a CDR L1 as set forth in SEQ ID NO:374, a CDR L2 as set forth in SEQ ID NO:375, and a CDR L3 as set forth in SEQ ID NO:376. In embodiments, the antibody includes a heavy chain variable domain including the sequence of SEQ ID NO:207 and a light chain variable domain including the sequence of SEQ ID NO:208. In embodiments, the antibody includes a heavy chain variable domain having the sequence of SEQ ID NO:207 and a light chain variable domain having the sequence of SEQ ID NO:208. In embodiments, the antibody is referred to herein as clone h6E6-072.

In embodiments, the antibody includes: a CDR H1 as set forth in SEQ ID NO:377, a CDR H2 as set forth in SEQ ID NO:378 and a CDR H3 as set forth in SEQ ID NO:379; and a CDR L1 as set forth in SEQ ID NO:380, a CDR L2 as set forth in SEQ ID NO:381, and a CDR L3 as set forth in SEQ ID NO:382. In embodiments, the antibody includes a heavy chain variable domain including the sequence of SEQ ID NO:217 and a light chain variable domain including the sequence of SEQ ID NO:218. In embodiments, the antibody includes a heavy chain variable domain having the sequence of SEQ ID NO:217 and a light chain variable domain having the sequence of SEQ ID NO:218. In embodiments, the antibody is referred to herein as clone h6E6-077.

In embodiments, the antibody includes: a CDR H1 as set forth in SEQ ID NO:383, a CDR H2 as set forth in SEQ ID NO:384 and a CDR H3 as set forth in SEQ ID NO:385; and a CDR L1 as set forth in SEQ ID NO:386, a CDR L2 as set forth in SEQ ID NO:387, and a CDR L3 as set forth in SEQ ID NO:388. In embodiments, the antibody includes a heavy chain variable domain including the sequence of SEQ ID NO:267 and a light chain variable domain including the sequence of SEQ ID NO:268. In embodiments, the antibody includes a heavy chain variable domain having the sequence of SEQ ID NO:267 and a light chain variable domain having the sequence of SEQ ID NO:268. In embodiments, the antibody is referred to herein as clone h6E6-102.

In embodiments, the antibody includes: a CDR H1 as set forth in SEQ ID NO:389, a CDR H2 as set forth in SEQ ID NO:390 and a CDR H3 as set forth in SEQ ID NO:391; and a CDR L1 as set forth in SEQ ID NO:392, a CDR L2 as set forth in SEQ ID NO:393, and a CDR L3 as set forth in SEQ ID NO:394. In embodiments, the antibody includes a heavy chain variable domain including the sequence of SEQ ID NO:277 and a light chain variable domain including the sequence of SEQ ID NO:278. In embodiments, the antibody includes a heavy chain variable domain having the sequence of SEQ ID NO:277 and a light chain variable domain having the sequence of SEQ ID NO:278. In embodiments, the antibody is referred to herein as clone h6E6-107.

In embodiments, the antibody includes: a CDR H1 as set forth in SEQ ID NO:395, a CDR H2 as set forth in SEQ ID NO:396 and a CDR H3 as set forth in SEQ ID NO:397; and a CDR L1 as set forth in SEQ ID NO:398, a CDR L2 as set forth in SEQ ID NO:399, and a CDR L3 as set forth in SEQ ID NO:400. In embodiments, the antibody includes a heavy chain variable domain including the sequence of SEQ ID NO:195 and a light chain variable domain including the sequence of SEQ ID NO: 196. In embodiments, the antibody includes a heavy chain variable domain having the sequence of SEQ ID NO:195 and a light chain variable domain having the sequence of SEQ ID NO:196. In embodiments, the antibody is referred to herein as clone h6E6-066.

In embodiments, the antibody is a humanized antibody. In embodiments, the antibody is a Fab′ fragment. In embodiments, the antibody is a single chain antibody (scFv). In embodiments, the antibody is a chimeric antibody. In embodiments, the antibody is an IgG. In embodiments, the antibody is an IgG1 or an IgG2. In embodiments, the antibody is an IgG1. In embodiments, the antibody is an IgG2.

In embodiments, the antibody is capable of binding a ROR2 protein. In embodiments, the ROR2 protein includes the amino acid sequence of SEQ ID NO:34, SEQ ID NO:38, SEQ ID NO:41, or SEQ ID NO:42. In embodiments, the ROR2 protein includes the amino acid sequence of SEQ ID NO:34. In embodiments, the ROR2 protein includes the amino acid sequence of SEQ ID NO:38. In embodiments, the ROR2 protein includes the amino acid sequence of SEQ ID NO:41 In embodiments, the ROR2 protein includes the amino acid sequence of SEQ ID NO:42. In embodiments, the ROR2 protein includes the amino acid sequence of SEQ ID NO:36 or SEQ ID NO:40. In embodiments, the ROR2 protein includes the amino acid sequence of SEQ ID NO:36. In embodiments, the ROR2 protein includes the amino acid sequence of SEQ ID NO:40.

In embodiments, the antibody is capable of binding the extracellular domain of the ROR2 protein. In embodiments, the extracellular domain includes the amino acid sequence of SEQ ID NO:38. In embodiments, the extracellular domain includes the amino acid sequence of SEQ ID NO:40. In embodiments, the ROR2 protein includes a histidine at a position corresponding to position 349 or an aspartic acid at a position corresponding to 354 of SEQ ID NO:38.

In embodiments, the extracellular domain is a Kringle domain. In embodiments, the Kringle domain includes the amino acid sequence of SEQ ID NO:41.

In embodiments, the ROR2 protein is expressed on a cell. In embodiments, the cell is a cancer cell. In embodiments, the cancer cell a breast cancer cell, an ovarian cancer cell, a pancreatic cancer cell, a cervical cancer cell, a gastric cancer cell, a renal cancer cell, a head and neck cancer cell, a bone cancer cell, a skin cancer cell or a prostate cancer cell. In embodiments, the cancer cell is a breast cancer cell. In embodiments, the cancer cell is an ovarian cancer cell. In embodiments, the cancer cell is a pancreatic cancer cell. In embodiments, the cancer cell is a cervical cancer cell. In embodiments, the cancer cell is a gastric cancer cell. In embodiments, the cancer cell is a renal cancer cell. In embodiments, the cancer cell is a head and neck cancer cell. In embodiments, the cancer cell is a bone cancer cell. In embodiments, the cancer cell is a skin cancer cell. In embodiments, the cancer cell is a prostate cancer cell.

In embodiments, the antibody is capable of binding the ROR2 protein with an equilibrium dissociation constant (KD) from about 0.02 nM to about 6 nM. In embodiments, the KD is from about 0.03 nM to about 6 nM. In embodiments, the KD is from about 0.04 nM to about 6 nM. In embodiments, the KD is from about 0.05 nM to about 6 nM. In embodiments, the KD is from about 0.06 nM to about 6 nM. In embodiments, the KD is from about 0.07 nM to about 6 nM. In embodiments, the KD is from about 0.08 nM to about 6 nM. In embodiments, the KD is from about 0.09 nM to about 6 nM. In embodiments, the KD is from about 0.1 nM to about 6 nM. In embodiments, the KD is from about 0.2 nM to about 6 nM. In embodiments, the KD is from about 0.3 nM to about 6 nM. In embodiments, the KD is from about 0.4 nM to about 6 nM. In embodiments, the KD is from about 0.5 nM to about 6 nM. In embodiments, the KD is from about 0.6 nM to about 6 nM. In embodiments, the KD is from about 0.7 nM to about 6 nM. In embodiments, the KD is from about 0.8 nM to about 6 nM. In embodiments, the KD is from about 0.9 nM to about 6 nM. In embodiments, the KD is from about 1 nM to about 6 nM. In embodiments, the KD is from about 2 nM to about 6 nM. In embodiments, the KD is from about 3 nM to about 6 nM. In embodiments, the KD is from about 4 nM to about 6 nM. In embodiments, the KD is from about 5 nM to about 6 nM.

In embodiments, the KD is from about 0.02 nM to about 5 nM. In embodiments, the KD is from about 0.02 nM to about 4 nM. In embodiments, the KD is from about 0.02 nM to about 3 nM. In embodiments, the KD is from about 0.02 nM to about 2 nM. In embodiments, the KD is from about 0.02 nM to about 1 nM. In embodiments, the KD is from about 0.02 nM to about 0.9 nM. In embodiments, the KD is from about 0.02 nM to about 0.8 nM. In embodiments, the KD is from about 0.02 nM to about 0.7 nM. In embodiments, the KD is from about 0.02 nM to about 0.6 nM. In embodiments, the KD is from about 0.02 nM to about 0.5 nM. In embodiments, the KD is from about 0.02 nM to about 0.4 nM. In embodiments, the KD is from about 0.02 nM to about 0.3 nM. In embodiments, the KD is from about 0.02 nM to about 0.2 nM. In embodiments, the KD is from about 0.02 nM to about 0.1 nM. In embodiments, the KD is from about 0.02 nM to about 0.09 nM. In embodiments, the KD is from about 0.02 nM to about 0.08 nM. In embodiments, the KD is from about 0.02 nM to about 0.07 nM. In embodiments, the KD is from about 0.02 nM to about 0.06 nM. In embodiments, the KD is from about 0.02 nM to about 0.05 nM. In embodiments, the KD is from about 0.02 nM to about 0.04 nM. In embodiments, the KD is from about 0.02 nM to about 0.03 nM.

In embodiments, the antibody is capable of binding the ROR2 protein with an equilibrium dissociation constant (KD) from 0.02 nM to 6 nM. In embodiments, the KD is from 0.03 nM to 6 nM. In embodiments, the KD is from 0.04 nM to 6 nM. In embodiments, the KD is from 0.05 nM to 6 nM. In embodiments, the KD is from 0.06 nM to 6 nM. In embodiments, the KD is from 0.07 nM to 6 nM. In embodiments, the KD is from 0.08 nM to 6 nM. In embodiments, the KD is from 0.09 nM to 6 nM. In embodiments, the KD is from 0.1 nM to 6 nM. In embodiments, the KD is from 0.2 nM to 6 nM. In embodiments, the KD is from 0.3 nM to 6 nM. In embodiments, the KD is from 0.4 nM to 6 nM. In embodiments, the KD is from 0.5 nM to 6 nM. In embodiments, the KD is from 0.6 nM to 6 nM. In embodiments, the KD is from 0.7 nM to 6 nM. In embodiments, the KD is from 0.8 nM to 6 nM. In embodiments, the KD is from 0.9 nM to 6 nM. In embodiments, the KD is from 1 nM to 6 nM. In embodiments, the KD is from 2 nM to 6 nM. In embodiments, the KD is from 3 nM to 6 nM. In embodiments, the KD is from 4 nM to 6 nM. In embodiments, the KD is from 5 nM to 6 nM.

In embodiments, the KD is from 0.02 nM to 5 nM. In embodiments, the KD is from 0.02 nM to 4 nM. In embodiments, the KD is from 0.02 nM to 3 nM. In embodiments, the KD is from 0.02 nM to 2 nM. In embodiments, the KD is from 0.02 nM to 1 nM. In embodiments, the KD is from 0.02 nM to 0.9 nM. In embodiments, the KD is from 0.02 nM to 0.8 nM. In embodiments, the KD is from 0.02 nM to 0.7 nM. In embodiments, the KD is from 0.02 nM to 0.6 nM. In embodiments, the KD is from 0.02 nM to 0.5 nM. In embodiments, the KD is from 0.02 nM to 0.4 nM. In embodiments, the KD is from 0.02 nM to 0.3 nM. In embodiments, the KD is from 0.02 nM to 0.2 nM. In embodiments, the KD is from 0.02 nM to 0.1 nM. In embodiments, the KD is from 0.02 nM to 0.09 nM. In embodiments, the KD is from 0.02 nM to 0.08 nM. In embodiments, the KD is from 0.02 nM to 0.07 nM. In embodiments, the KD is from 0.02 nM to 0.06 nM. In embodiments, the KD is from 0.02 nM to 0.05 nM. In embodiments, the KD is from 0.02 nM to 0.04 nM. In embodiments, the KD is from 0.02 nM to 0.03 nM.

In embodiments, the antibody is capable of binding the ROR2 protein with an equilibrium dissociation constant (KD) of about 0.024 nM. In embodiments, the antibody is capable of binding the ROR2 protein with an equilibrium dissociation constant (KD) of about 0.079 nM. In embodiments, the antibody is capable of binding the ROR2 protein with an equilibrium dissociation constant (KD) of about 0.155 nM. In embodiments, the antibody is capable of binding the ROR2 protein with an equilibrium dissociation constant (KD) of about 0.165 nM. In embodiments, the antibody is capable of binding the ROR2 protein with an equilibrium dissociation constant (KD) of about 0.186 nM. In embodiments, the antibody is capable of binding the ROR2 protein with an equilibrium dissociation constant (KD) of about 0.187 nM. In embodiments, the antibody is capable of binding the ROR2 protein with an equilibrium dissociation constant (KD) of about 0.261 nM. In embodiments, the antibody is capable of binding the ROR2 protein with an equilibrium dissociation constant (KD) of about 0.318 nM. In embodiments, the antibody is capable of binding the ROR2 protein with an equilibrium dissociation constant (KD) of about 0.319 nM. In embodiments, the antibody is capable of binding the ROR2 protein with an equilibrium dissociation constant (KD) of about 0.348 nM. In embodiments, the antibody is capable of binding the ROR2 protein with an equilibrium dissociation constant (KD) of about 0.353 nM. In embodiments, the antibody is capable of binding the ROR2 protein with an equilibrium dissociation constant (KD) of about 0.362 nM. In embodiments, the antibody is capable of binding the ROR2 protein with an equilibrium dissociation constant (KD) of about 0.380 nM. In embodiments, the antibody is capable of binding the ROR2 protein with an equilibrium dissociation constant (KD) of about 0.422 nM. In embodiments, the antibody is capable of binding the ROR2 protein with an equilibrium dissociation constant (KD) of about 0.463 nM. In embodiments, the antibody is capable of binding the ROR2 protein with an equilibrium dissociation constant (KD) of about 0.475 nM.

In embodiments, the antibody is capable of binding the ROR2 protein with an equilibrium dissociation constant (KD) of 0.024 nM. In embodiments, the antibody is capable of binding the ROR2 protein with an equilibrium dissociation constant (KD) of 0.079 nM. In embodiments, the antibody is capable of binding the ROR2 protein with an equilibrium dissociation constant (KD) of 0.155 nM. In embodiments, the antibody is capable of binding the ROR2 protein with an equilibrium dissociation constant (KD) of 0.165 nM. In embodiments, the antibody is capable of binding the ROR2 protein with an equilibrium dissociation constant (KD) of 0.186 nM. In embodiments, the antibody is capable of binding the ROR2 protein with an equilibrium dissociation constant (KD) of 0.187 nM. In embodiments, the antibody is capable of binding the ROR2 protein with an equilibrium dissociation constant (KD) of 0.261 nM. In embodiments, the antibody is capable of binding the ROR2 protein with an equilibrium dissociation constant (KD) of 0.318 nM. In embodiments, the antibody is capable of binding the ROR2 protein with an equilibrium dissociation constant (KD) of 0.319 nM. In embodiments, the antibody is capable of binding the ROR2 protein with an equilibrium dissociation constant (KD) of 0.348 nM. In embodiments, the antibody is capable of binding the ROR2 protein with an equilibrium dissociation constant (KD) of 0.353 nM. In embodiments, the antibody is capable of binding the ROR2 protein with an equilibrium dissociation constant (KD) of 0.362 nM. In embodiments, the antibody is capable of binding the ROR2 protein with an equilibrium dissociation constant (KD) of 0.380 nM. In embodiments, the antibody is capable of binding the ROR2 protein with an equilibrium dissociation constant (KD) of 0.422 nM. In embodiments, the antibody is capable of binding the ROR2 protein with an equilibrium dissociation constant (KD) of 0.463 nM. In embodiments, the antibody is capable of binding the ROR2 protein with an equilibrium dissociation constant (KD) of 0.475 nM.

In embodiments, the antibody is attached to a therapeutic agent. In embodiments, the antibody is attached to a diagnostic agent.

In another aspect is provided an anti-ROR2 antibody, wherein the anti-ROR2 antibody binds the same epitope as an anti-ROR2 antibody provided herein including embodiments thereof.

### Chimeric Antigen Receptor Proteins

As described above, the CDRs of the heavy chain variable (VH) domain and the CDRs of the light chain variable (VL) domain provided herein including embodiments thereof, may each independently form part of an antibody, a fragment of an antibody, or a a chimeric antigen receptor or bispecific antibody. Provided herein are, inter alia, chimeric antigen receptors and bispecific antibodies, which include the light chain variable (VL) domain and/or the heavy chain variable (VH) domain as provided herein and are therefore capable of binding human ROR2 effectively and efficiently. The antibody region of the chimeric antigen receptor may include any of the light chain and heavy chain variable domains provided herein including embodiments thereof. The light chain variable (VL) domain and/or the heavy chain variable (VH) domain as provided herein may form part of a chimeric antigen receptor. Thus, in an aspect is provided a chimeric antigen receptor including (i) an antibody region including any one of the light chain variable domain and heavy chain variable domain pairings provided herein including embodiments thereof; and (ii) a transmembrane domain.

An “antibody region” as provided herein refers to a monovalent or multivalent protein moiety that forms part of the recombinant protein (e.g., CAR, bispecific antibody) provided herein including embodiments thereof. A person of ordinary skill in the art will therefore immediately recognize that the antibody region is a protein moiety capable of binding an antigen (epitope). Thus, the antibody region provided herein may include a domain of an antibody (e.g., a light chain variable (VL) domain, a heavy chain variable (VH) domain) or a fragment of an antibody (e.g., Fab). In embodiments, the antibody region is a protein conjugate. A “protein conjugate” as provided herein refers to a construct consisting of more than one polypeptide, wherein the polypeptides are bound together covalently or non-covalently. In embodiments, the polypeptides of a protein conjugate are encoded by one nucleic acid molecule. In embodiments, the polypeptides of a protein conjugate are encoded by different nucleic acid molecules. In embodiments, the polypeptides are connected through a linker. In embodiments, the polypeptides are connected through a chemical linker. In embodiments, the antibody region is an scFv. The antibody region may include a light chain variable (VL) domain and/or a heavy chain variable (VH) domain. In embodiments, the antibody region includes a light chain variable (VL) domain. In embodiments, the antibody region includes a heavy chain variable (VH) domain.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein including embodiments thereof are contemplated. Non-limiting examples of transmembrane domains include the transmembrane domains of CD28, CD8, CD4 or CD3-zeta (also known as CD247).

In embodiments, the transmembrane domain is a CD4 transmembrane domain. In embodiments, the transmembrane domain is a CD28 transmembrane domain. The term “CD28 transmembrane domain” as provided herein includes any of the recombinant or naturally-occurring forms of the transmembrane domain of CD28, or variants or homologs thereof that maintain CD28 transmembrane domain activity (e.g. within at least 50%, 80%, 90%, 95%, 96%, 97%, 98%, 99% or 100% activity compared to the CD28 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 transmembrane domain polypeptide. In embodiments, CD28 is the protein as identified by the NCBI sequence reference GI:340545506, homolog or functional fragment thereof.

In embodiments, the transmembrane domain is a CD8 transmembrane domain. The term “CD8 transmembrane domain” as provided herein includes any of the recombinant or naturally-occurring forms of the transmembrane domain of CD8, or variants or homologs thereof that maintain CD8 transmembrane domain activity (e.g. within at least 50%, 80%, 90%, 95%, 96%, 97%, 98%, 99% or 100% activity compared to the CD8 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD8 transmembrane domain polypeptide. In embodiments, CD8 is the protein as identified by the NCBI sequence reference GI:225007534, homolog or functional fragment thereof.

In embodiments, the transmembrane domain is a CD4 transmembrane domain. The term “CD4 transmembrane domain” as provided herein includes any of the recombinant or naturally-occurring forms of the transmembrane domain of CD4, or variants or homologs thereof that maintain CD4 transmembrane domain activity (e.g. within at least 50%, 80%, 90%, 95%, 96%, 97%, 98%, 99% or 100% activity compared to the CD4 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD4 transmembrane domain polypeptide. In embodiments, CD4 is the protein as identified by the NCBI sequence reference GI:303522473, homolog or functional fragment thereof.

In embodiments, the transmembrane domain is a CD3-zeta (CD247) transmembrane domain. The term “CD3-zeta transmembrane domain” as provided herein includes any of the recombinant or naturally-occurring forms of the transmembrane domain of CD3-zeta (CD247), or variants or homologs thereof that maintain CD3-zeta (CD247) transmembrane domain activity (e.g. within at least 50%, 80%, 90%, 95%, 96%, 97%, 98%, 99% or 100% activity compared to the CD3-zeta (CD247) transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3-zeta (CD247) transmembrane domain polypeptide. In embodiments, CD3-zeta is the protein as identified by the NCBI sequence reference GI: 166362721, homolog or functional fragment thereof.

In embodiments, the chimeric antigen receptor further includes an intracellular T-cell signaling domain. An “intracellular T-cell signaling domain” as provided herein includes amino acid sequences capable of providing primary signaling in response to binding of an antigen to the antibody region provided herein including embodiments thereof. In embodiments, the signaling of the intracellular T-cell signaling domain results in activation of the T cell expressing the same. In embodiments, the signaling of the intracellular T-cell signaling domain results in proliferation (cell division) of the T cell expressing the same. In embodiments, the signaling of the intracellular T-cell signaling domain results expression by said T cell of proteins known in the art to characteristic of activated T cell (e.g., CTLA-4 (CD152), PD-1 (CD274), CD28, CD69). In embodiments, the intracellular T-cell signaling domain is a CD3 ζ intracellular T-cell signaling domain.

In embodiments, the chimeric antigen receptor further includes an intracellular co-stimulatory T-cell signaling domain. An “intracellular co-stimulatory signaling domain” as provided herein includes amino acid sequences capable of providing co-stimulatory signaling in response to binding of an antigen to the antibody region provided herein including embodiments thereof. In embodiments, the signaling of the co-stimulatory signaling domain results in production of cytokines and proliferation of the T cell expressing the same. In embodiments, the intracellular co-stimulatory signaling domain is a CD28 intracellular co-stimulatory signaling domain, a 4-1BB (CD137) intracellular co-stimulatory signaling domain, or an OX-40 (CD134) intracellular co-stimulatory signaling domain. In embodiments, the intracellular co-stimulatory signaling domain is a CD28 intracellular co-stimulatory signaling domain. In embodiments, the intracellular co-stimulatory signaling domain is a 4-1BB (CD137) intracellular co-stimulatory signaling domain. In embodiments, the intracellular co-stimulatory signaling domain is an OX-40 (CD134) intracellular co-stimulatory signaling domain.

In embodiments, the antibody region includes an Fc domain. In embodiments, the antibody region includes a spacer region. In embodiments, the spacer region is between the transmembrane domain and the antibody region. A “spacer region” as provided herein is a polypeptide connecting the antibody region with the transmembrane domain. In embodiments, the spacer region connects the heavy chain constant region with the transmembrane domain. In embodiments, the spacer region includes an Fc region. In embodiments, the spacer region is an Fc region. Examples of spacer regions contemplated for the compositions provided herein include without limitation, immunoglobulin molecules or fragments thereof (e.g., IgG1, IgG2, IgG3, IgG4) and immunoglobulin molecules or fragments thereof (e.g., IgG1, IgG2, IgG3, IgG4) including mutations affecting Fc receptor binding. In embodiments, the spacer region is a hinge region.

The term “CTLA-4” as referred to herein includes any of the recombinant or naturally-occurring forms of the cytotoxic T-lymphocyte-associated protein 4 protein, also known as CD152 (cluster of differentiation 152), or variants or homologs thereof that maintain CTLA-4 activity (e.g. within at least 50%, 80%. 90%, 95%, 96%, 97%, 98%, 99% or 100% activity compared to CTLA-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 protein. In embodiments, the CTLA-4 protein is substantially identical to the protein identified by the UniProt reference number P16410 or a variant or homolog having substantial identity thereto.

The term “CD28” as referred to herein includes any of the recombinant or naturally-occurring forms of the Cluster of Differentiation 28 protein, or variants or homologs thereof that maintain CD28 activity (e.g. within at least 50%, 80%, 90%, 95%, 96%, 97%, 98%, 99% or 100% activity compared to CD28). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 protein. In embodiments, the CD28 protein is substantially identical to the protein identified by the UniProt reference number P10747 or a variant or homolog having substantial identity thereto.

The term “CD69” as referred to herein includes any of the recombinant or naturally-occurring forms of the Cluster of Differentiation 69 protein, or variants or homologs thereof that maintain CD69 activity (e.g. within at least 50%, 80%, 90%, 95%, 96%, 97%, 98%, 99% or 100% activity compared to CD69). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD69 protein. In embodiments, the CD69 protein is substantially identical to the protein identified by the UniProt reference number Q07108 or a variant or homolog having substantial identity thereto.

The term “4-1BB” as referred to herein includes any of the recombinant or naturally-occurring forms of the 4-11B protein, also known as tumor necrosis factor receptor superfamily member 9 (TNFRSF9). Cluster of Differentiation 137 (CD137) and induced by lymphocyte activation (ILA), or variants or homologs thereof that maintain 4-1BB activity (e.g. within at least 50%, 80%, 90%, 95%, 96%, 97%, 98%, 99% or 100% activity compared to 4-1BB). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the 4-1BB protein is substantially identical to the protein identified by the UniProt reference number Q07011 or a variant or homolog having substantial identity thereto.

In another aspect is provided a chimeric antigen receptor including (i) an anti-ROR2 antibody provided herein including embodiments thereof and (ii) a transmembrane domain.

In embodiments, the antibody includes: a CDR H1 as set forth in SEQ ID NO:305, a CDR H2 as set forth in SEQ ID NO:306 and a CDR H3 as set forth in SEQ ID NO:307; and a CDR L1 as set forth in SEQ ID NO:308, a CDR L2 as set forth in SEQ ID NO: 309, and a CDR L3 as set forth in SEQ ID NO:310. In embodiments, the antibody includes a heavy chain variable domain including the sequence of SEQ ID NO:203 and a light chain variable domain including the sequence of SEQ ID NO:204. In embodiments, the antibody includes a heavy chain variable domain having the sequence of SEQ ID NO:203 and a light chain variable domain having the sequence of SEQ ID NO:204.

In embodiments, the antibody includes: a CDR H1 as set forth in SEQ ID NO:311, a CDR H2 as set forth in SEQ ID NO:312 and a CDR H3 as set forth in SEQ ID NO: 313; and a CDR L1 as set forth in SEQ ID NO:314, a CDR L2 as set forth in SEQ ID NO: 315, and a CDR L3 as set forth in SEQ ID NO:316. In embodiments, the antibody includes a heavy chain variable domain including the sequence of SEQ ID NO:295 and a light chain variable domain including the sequence of SEQ ID NO:296. In embodiments, the antibody includes a heavy chain variable domain having the sequence of SEQ ID NO:295 and a light chain variable domain having the sequence of SEQ ID NO:296.

In embodiments, the chimeric antigen receptor further includes an intracellular T-cell signaling domain. In embodiments, the intracellular T-cell signaling domain is a CD3 ζ intracellular T-cell signaling domain.

In embodiments, the chimeric antigen receptor further includes an intracellular co-stimulatory T-cell signaling domain. In embodiments, the intracellular co-stimulatory T-cell signaling domain is a CD28 intracellular co-stimulatory signaling domain, a 4-1BB intracellular co-stimulatory signaling domain, an intracellular co-stimulatory signaling domain, or an OX-40 (CD134) intracellular co-stimulatory signaling domain. In embodiments, the intracellular co-stimulatory T-cell signaling domain is a CD28 intracellular co-stimulatory signaling domain. In embodiments, the intracellular co-stimulatory T-cell signaling domain is a 4-1BB intracellular co-stimulatory signaling domain. In embodiments, the intracellular co-stimulatory T-cell signaling domain is an intracellular co-stimulatory signaling domain. In embodiments, the intracellular co-stimulatory T-cell signaling domain is an OX-40 (CD134)intracellular co-stimulatory signaling domain.

In embodiments, the chimeric antigen receptor further includes a spacer region. In embodiments, the spacer region further includes a hinge region. In embodiments, the chimeric antigen receptor further includes a linker domain. In embodiments, the chimeric antigen receptor further includes a heavy chain constant domain.

### Bispecific Antibodies

The light chain variable (VL) domain and the heavy chain variable (VH) domain as provided herein may form part of a bispecific antibody. Thus, the second antibody region may include any of the light chain and/or heavy chain variable domains provided herein including embodiments thereof.

The term “effector cell ligand” as provided herein refers to a cell surface molecule expressed on an effector cell of the immune system (e.g., a cytotoxic T cell, a helper T cell, a B cell, a natural killer cell). Upon binding of the first antibody region to the effector cell ligand expressed on the effector cell, the effector cell is activated and able to exert its function (e.g., selective killing or eradication of malignant, infected or otherwise unhealthy cells). In embodiments, the effector cell ligand is a CD3 protein. In embodiments, the effector cell ligand is a CD16 protein. In embodiments, the effector cell ligand is a CD32 protein. In embodiments, the effector cell ligand is a NKp46 protein. The first antibody region as provided herein may be an antibody, an antibody variant, a fragment of an antibody or a fragment of an antibody variant.

A “CD3 protein” as referred to herein includes any of the recombinant or naturally-occurring forms of the Cluster of Differentiation 3 (CD3) proteins or variants or homologs thereof that comprise the CD3 complex that mediates signal transduction and maintains CD3 complex activity (e.g. within at least 50%, 80%. 90%, 95%, 96%, 97%, 98%, 99% or 100% activity compared to the CD3 complex). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3 proteins in the CD3 complex.

A “CD16 protein” as referred to herein includes any of the recombinant or naturally-occurring forms of the Cluster of Differentiation 16 (CD16) protein, also known as low affinity immunoglobulin gamma Fc region receptor III-A, or variants or homologs thereof that maintain CD16 activity (e.g. within at least 50%, 80%, 90%, 95%, 96%, 97%, 98%, 99% or 100% activity compared to CD16). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD16 protein. In embodiments, the CD16 protein is substantially identical to the protein identified by the UniProt reference number P08637 or a variant or homolog having substantial identity thereto.

A “CD32 protein” as referred to herein includes any of the recombinant or naturally-occurring forms of the Cluster of Differentiation 32 (CD32) protein, also known as low affinity immunoglobulin gamma Fc region receptor II-A, or variants or homologs thereof that maintain CD32 activity (e.g. within at least 50%, 80%, 90%, 95%, 96%, 97%, 98%, 99% or 100% activity compared to CD32). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD32 protein. In embodiments, the CD32 protein is substantially identical to the protein identified by the UniProt reference number P12318 or a variant or homolog having substantial identity thereto.

A “NKp46 protein” as referred to herein includes any of the recombinant or naturally-occurring forms of the NKp46 protein, also known as natural cytotoxicity triggering receptor 1, or variants or homologs thereof that maintain NKp46 activity (e.g. within at least 50%, 80%, 90%, 95%, 96%, 97%, 98%, 99% or 100% activity compared to NKp46). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring NKp46 protein. In embodiments, the NKp46 protein is substantially identical to the protein identified by the UniProt reference number 076036 or a variant or homolog having substantial identity thereto.

### Methods of Treatment

The compositions (e.g., the anti-ROR2 antibodies CARs and bispecific antibodies) provided herein, including embodiments thereof, are contemplated as providing effective treatments for diseases such as cancer (e.g., breast cancer).

Thus, in an aspect is provided a method of treating cancer in a subject in need thereof, the method including administering to a subject a therapeutically effective amount of an antibody as provided herein including embodiments thereof. In another aspect is provided a method of inhibiting metastasis of a ROR2 expressing cancer in a subject in need thereof, the method including administering to a subject a therapeutically effective amount of an antibody provided herein including embodiments thereof.

In another aspect is provided a method of treating cancer in a subject in need thereof, the method including administering a therapeutically effective amount of a chimeric antigen receptor provided herein including embodiments thereof to a subject.

In embodiments, the cancer is a solid tumor malignancy. In embodiments, the cancer is breast cancer, ovarian cancer, pancreatic cancer, cervical cancer, gastric cancer, renal cancer, head and neck cancer, bone cancer, skin cancer or prostate cancer. In embodiments, the cancer is breast cancer. In embodiments, the cancer is ovarian cancer. In embodiments, the cancer is pancreatic cancer. In embodiments, the cancer is cervical cancer. In embodiments, the cancer is gastric cancer. In embodiments, the cancer is renal cancer. In embodiments, the cancer is head and neck cancer. In embodiments, the cancer is bone cancer. In embodiments, the cancer is skin cancer. In embodiments, the cancer is prostate cancer.

In an aspect is provided a method of treating cancer in a subject in need thereof, the method including administering to a subject a therapeutically effective amount of an antibody provided herein including embodiments thereof.

In embodiments, the cancer is breast cancer, ovarian cancer, pancreatic cancer, cervical cancer, gastric cancer, renal cancer, head and neck cancer, bone cancer, skin cancer or prostate cancer. In embodiments, the cancer is breast cancer. In embodiments, the cancer is ovarian cancer. In embodiments, the cancer is pancreatic cancer. In embodiments, the cancer is cervical cancer. In embodiments, the cancer is gastric cancer. In embodiments, the cancer is renal cancer. In embodiments, the cancer is head and neck cancer. In embodiments, the cancer is bone cancer. In embodiments, the cancer is skin cancer. In embodiments, the cancer is prostate cancer.

In another aspect is provided a method of detecting a ROR2 expressing cell, the method including (i) contacting a ROR2-expressing cell with an antibody provided herein including embodiments thereof; and (ii) detecting binding of the antibody to a ROR2 protein expressed by the cell.

In embodiments, the antibody is attached to a detectable moiety.

In embodiments, ROR2-expressing cell is in a subject.

In embodiments, the subject is a subject having or being at risk of having cancer. In embodiments, the subject is a subject having. In embodiments, the subject is a subject being at risk of having cancer.

In embodiments, the cancer is breast cancer, ovarian cancer, pancreatic cancer, cervical cancer, gastric cancer, renal cancer, head and neck cancer, bone cancer, skin cancer or prostate cancer. In embodiments, the cancer is breast cancer. In embodiments, the cancer is ovarian cancer. In embodiments, the cancer is pancreatic cancer. In embodiments, the cancer is cervical cancer. In embodiments, the cancer is gastric cancer. In embodiments, the cancer is renal cancer. In embodiments, the cancer is head and neck cancer. In embodiments, the cancer is bone cancer. In embodiments, the cancer is skin cancer. In embodiments, the cancer is prostate cancer.

In embodiments, the contacting occurs in vitro.

In another aspect is provided a method of delivering a therapeutic agent to a ROR2 expressing cell, the method including contacting a ROR2 expressing cell with an antibody provided herein including embodiments thereof, wherein the antibody is attached to a therapeutic agent.

In embodiments, the therapeutic agent is an anti-cancer agent.

In embodiments, the ROR2-expressing cell is in a subject.

In embodiments, the subject is a subject having or being at risk of having cancer. In embodiments, the subject is a subject having. In embodiments, the subject is a subject being at risk of having cancer.

In embodiments, the cancer is breast cancer, ovarian cancer, pancreatic cancer, cervical cancer, gastric cancer, renal cancer, head and neck cancer, bone cancer, skin cancer or prostate cancer. In embodiments, the cancer is breast cancer. In embodiments, the cancer is ovarian cancer. In embodiments, the cancer is pancreatic cancer. In embodiments, the cancer is cervical cancer. In embodiments, the cancer is gastric cancer. In embodiments, the cancer is renal cancer. In embodiments, the cancer is head and neck cancer. In embodiments, the cancer is bone cancer. In embodiments, the cancer is skin cancer. In embodiments, the cancer is prostate cancer.

In embodiments, the contacting occurs in vitro.

In another aspect is provided a method of inhibiting migration of a ROR2-expressing cell, the method including contacting a ROR2 expressing cell with an antibody provided herein including embodiments thereof.

In embodiments, the ROR2 expressing cell is in a subject having or being at risk of having cancer. In embodiments, the subject is a subject having. In embodiments, the subject is a subject being at risk of having cancer.

In embodiments, the cancer is breast cancer, ovarian cancer, pancreatic cancer, cervical cancer, gastric cancer, renal cancer, head and neck cancer, bone cancer, skin cancer or prostate cancer. In embodiments, the cancer is breast cancer. In embodiments, the cancer is ovarian cancer. In embodiments, the cancer is pancreatic cancer. In embodiments, the cancer is cervical cancer. In embodiments, the cancer is gastric cancer. In embodiments, the cancer is renal cancer. In embodiments, the cancer is head and neck cancer. In embodiments, the cancer is bone cancer. In embodiments, the cancer is skin cancer. In embodiments, the cancer is prostate cancer.

In embodiments, the contacting occurs in vitro.

In embodiments, the ROR2 expressing cell is a cancer cell.

In embodiments, the cancer cell is a breast cancer cell, an ovarian cancer cell, a pancreatic cancer cell, a cervical cancer cell, a gastric cancer cell, a renal cancer cell, a head and neck cancer cell, a bone cancer cell, a skin cancer cell or a prostate cancer cell. In embodiments, the cancer cell is a breast cancer cell. In embodiments, the cancer cell is an ovarian cancer cell. In embodiments, the cancer cell is a pancreatic cancer cell. In embodiments, the cancer cell is a cervical cancer cell. In embodiments, the cancer cell is a gastric cancer cell. In embodiments, the cancer cell is a renal cancer cell. In embodiments, the cancer cell is a head and neck cancer cell. In embodiments, the cancer cell is a bone cancer cell. In embodiments, the cancer cell is a skin cancer cell. In embodiments, the cancer cell is a prostate cancer cell.

In another aspect is provided a method of treating cancer in a subject in need thereof the method including, administering a therapeutically effective amount of a chimeric antigen receptor provide herein including embodiments thereof.

In embodiments, the cancer is breast cancer, ovarian cancer, pancreatic cancer, cervical cancer, gastric cancer, renal cancer, head and neck cancer, bone cancer, skin cancer or prostate cancer. In embodiments, the cancer is breast cancer. In embodiments, the cancer is ovarian cancer. In embodiments, the cancer is pancreatic cancer. In embodiments, the cancer is cervical cancer. In embodiments, the cancer is gastric cancer. In embodiments, the cancer is renal cancer. In embodiments, the cancer is head and neck cancer. In embodiments, the cancer is bone cancer. In embodiments, the cancer is skin cancer. In embodiments, the cancer is prostate cancer.

### EXAMPLES

### Example 1

### Humanized ROR2 mAb

A Receptor tyrosine kinase-like orphan receptor 2 (ROR2) was a developmentally restricted receptor for certain Wnt factors, such as Wnt5a. A human ROR2 (interchangeably referred to as ROR2) was a 943-amino acid single-pass type I membrane protein with a calculated molecular weight of 104.8 kilodaltons (kDa). The ROR2 was highly conserved across several species with a 92% amino acid identity between the mouse and human proteins. The ROR2 might repress transcription of Wnt target genes and modulate Wnt signaling by sequestering canonical Wnt ligands, thereby serving as a tumor suppressor in different cell contexts. Recently, the ROR2 has been implicated in progression of numerous cancers including breast, ovarian, pancreatic, cervical, gastric, renal, head and neck, bone, skin and prostate. Due to the expression of the ROR2 in cancer cells, ROR2 has the potential to serve as a diagnostic and therapeutic target.

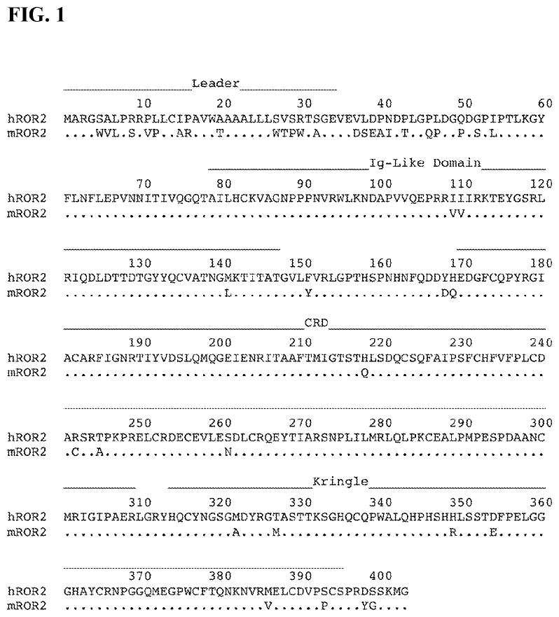

The monoclonal antibodies (mAb) produced, specifically targeted the extracellular portion of the human ROR2. For generatino of such antibodies, mice were immunized with recombinant protein of the extracellular portion (AA 1-403) of the ROR2 protein that included the Ig-like, cysteine rich domain (CRD) and Kringle domains as represented in FIG. 1. Immune stimulatory agents, such as a Freund's Complete Adjuvant was co-injected, because of the high degree of homology between the murine and human molecules, to maximize the generation of anti-human ROR2 antibodies. Hybridomas generated following fusion of splenocytes with a myeloma fusion partner were screened for expression of an anti-ROR2 mAb using an ELISA or flow cytometry to identify clones that produce antibody that specifically bind to the human ROR2. One mAb, designated 6E6, was chosen for further development and was humanized. The murine framework regions of both a immunoglobulin heavy chain variable region gene (IGHV) and a immunoglobulin kappa variable cluster (IGKV) were replaced with homologous framework regions of human IGHV and IGKV and the constant region was converted to the human IgG1. Sixteen humanized mAb were generated to retain binding specificity and reactivity with the human ROR2. These mAb are designated in this disclosure as mAbs N-LN or in the more familiar form h6E6-NNN and are listed in Table 1. All 16 mAb have a high affinity for binding to the ROR2, with binding affinities (Kd) ranging from 24 picomolar (pM) to 500 pM, as measured by surface plasmon resonance. Additionally, biochemical analyses of thermostability and aggregation demonstrate that all mAb have profiles that indicate they are quite stable. The sequences of the heavy and light chain variable regions are included in the informal sequence listing section of the application.

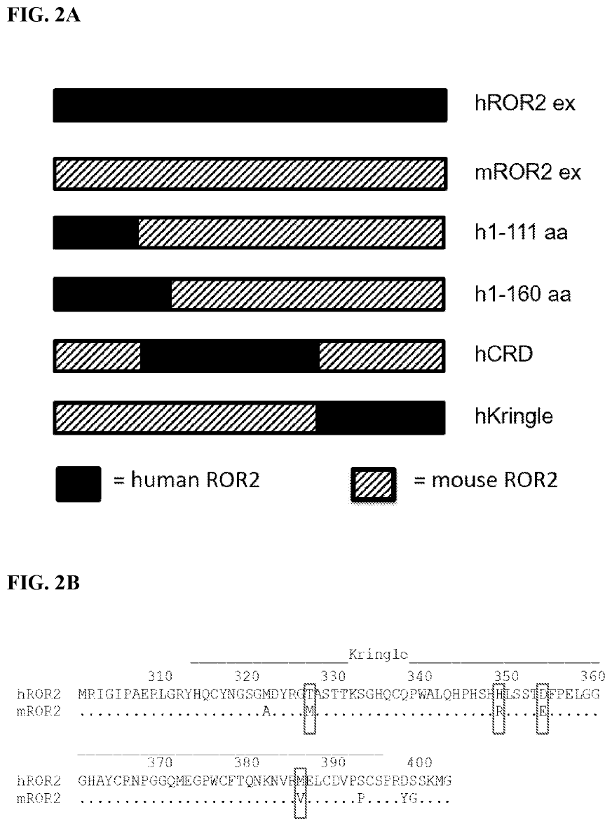

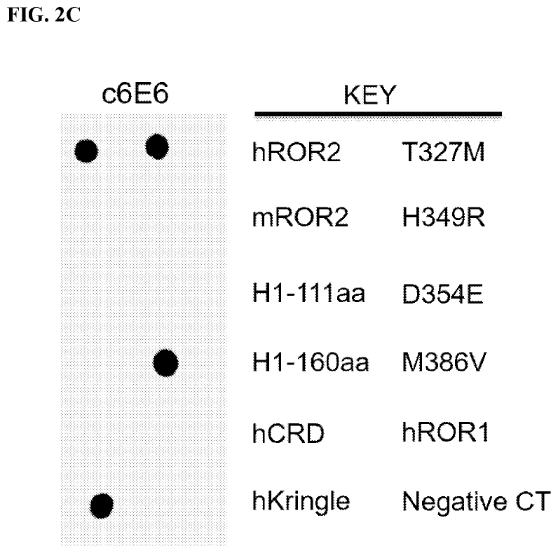

The parental 6E6 mouse an anti-human ROR2 mAb was previously shown to bind to the Kringle region of the human ROR2. It also required a histidine residue at position 349 and an aspartic acid at position 354 for binding to the Kringle domain of the human ROR2, as replacement of these amino acids with the murine molecule's corresponding arginine and glutamic acid residues, respectively, abrogated binding to the human ROR2. For this, a series of recombinant human/mouse hybrid proteins were developed that isolate specific domains of the human ROR2 protein. These recombinant proteins were used to identify the specific binding domains that each of the mAb target as shown in FIG. 2A. The 6E6 mAb binds to the Kringle domain of the human ROR2, but not the mouse ROR2 as shown in FIG. 2C. The human/mouse chimeric ROR2 recombinant proteins were usedto identify specific amino acids that are critical for the binding of the anti-ROR2 mAbs that bind within the Kringle domain as shown in FIG. 2B. For this, several recombinant human ROR2 proteins were produced the in which one of the four amino acids that differs between human and mouse ROR2 within the Kringle domain was replaced with the corresponding amino acid of the mouse ROR2. Assessment of binding of 6E6 indicates that this mAb requires a histidine residue at position 349 and aspartic acid at position 354 for binding to the Kringle domain of the human ROR2, as replacement of these amino acids with the murine molecule's arginine and glutamic acid residues, respectively, the 6E6 mAb no longer binds the recombinant ROR2 protein.

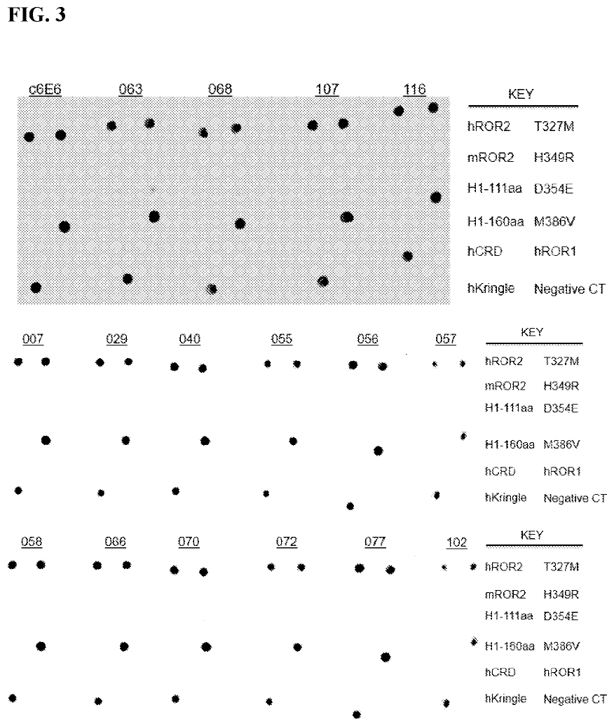

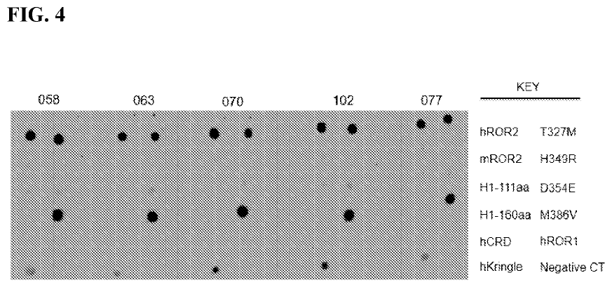

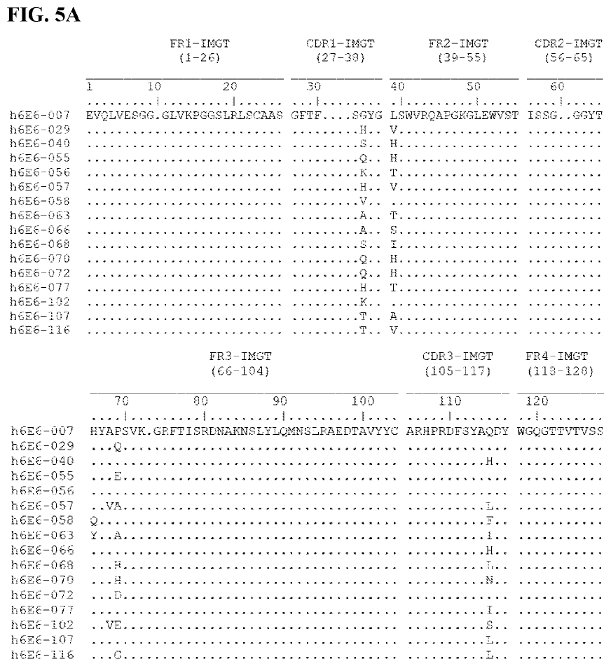

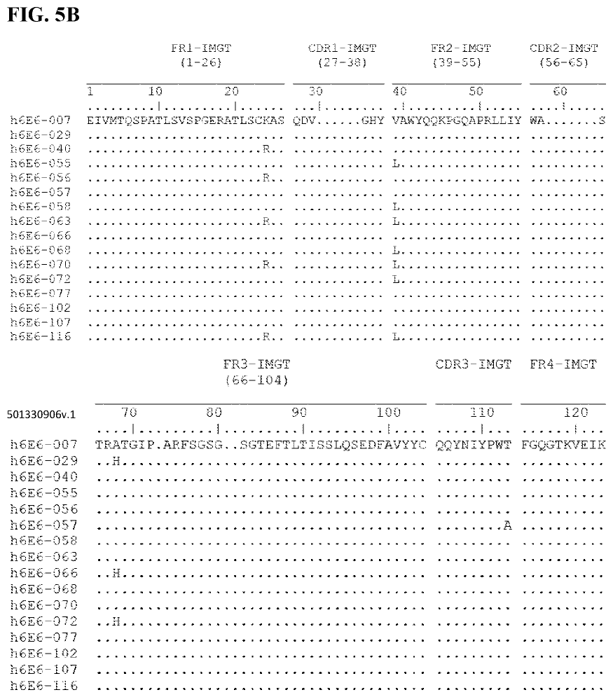

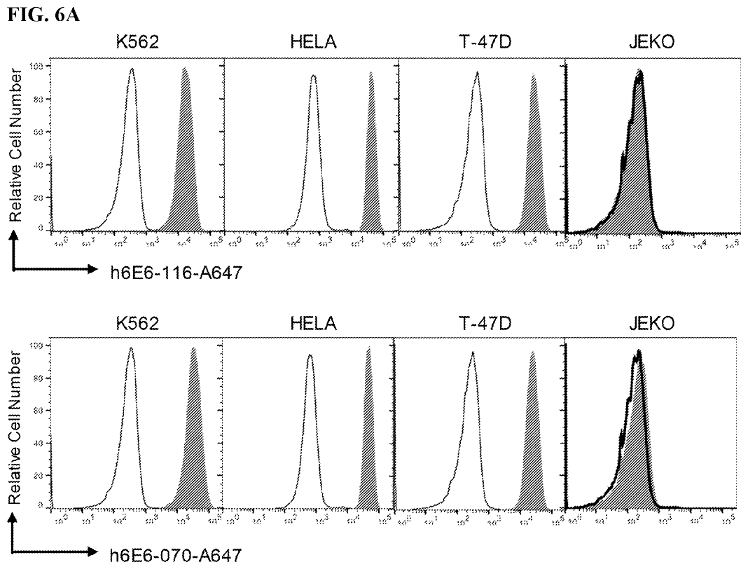

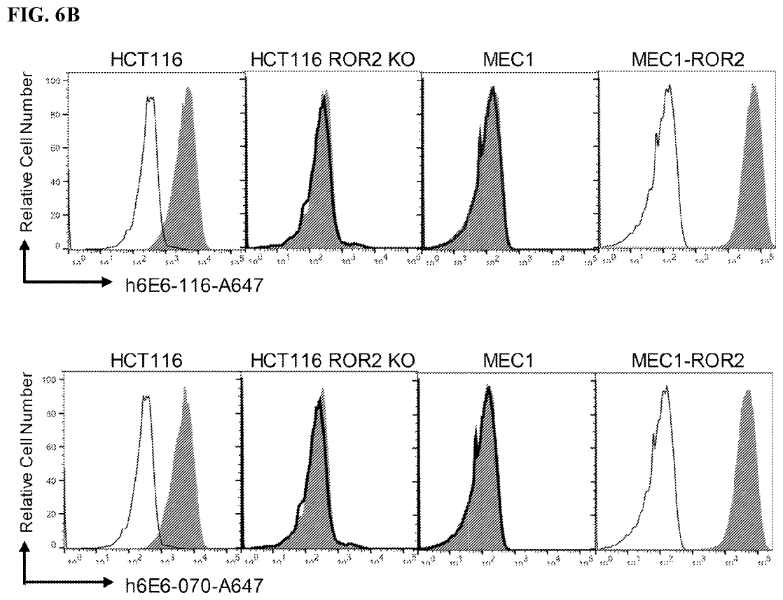

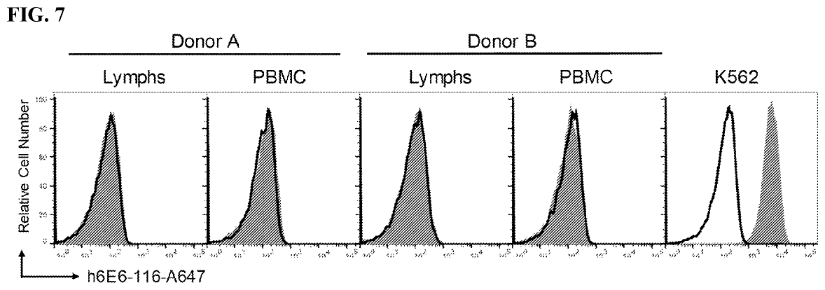

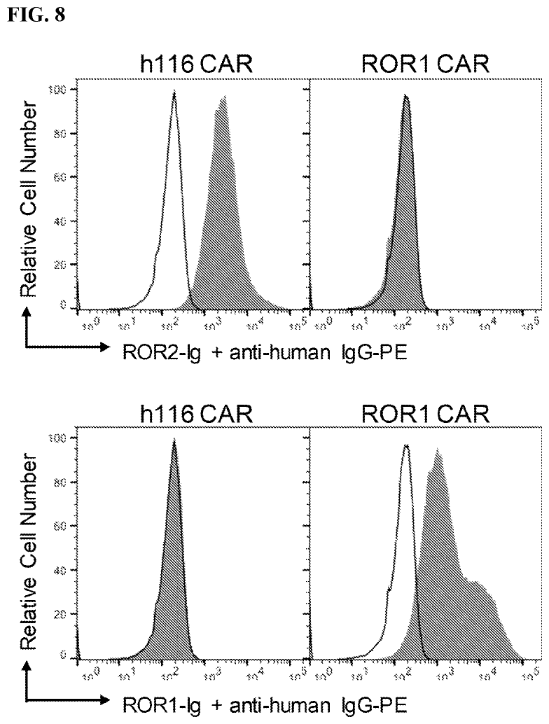

Each of the 16 humanized 6E6 (h6E6) mAb retain the same binding specificity as the parental 6E6 mAb, as they bind to the same determinant in the Kringle domain of human as shown in FIG. 3. Binding reactivity of each of the 16 h6E6 mAb were assessed using the same set of recombinant ROR2 proteins. Each of the sixteen humanized mAb retains the same binding pattern/profile as the parental 6E6 mAb, binding to human ROR2 and the human/mouse hybrid protein that contains the human Kringle domain, but not to the mouse ROR2 or any of the human/mouse hybrid proteins that contain the mouse Kringle domain. More importantly, all sixteen h6E6 mAb do not bind to the human ROR2 constructs in which the histidine residue at position 349 of the human ROR2 was replaced with arginine (H349R) or aspartic acid at position 354 was replaced with glutamic acid (D345E). Amino acid substitutions at positions 327 (T327M) and 386 (M386V) did not influence mAb binding, and none of the humanized mAb bound to a recombinant ROR1 or the negative control supernatant.

Based on these findings, it is apparent that the these humanized anti-human ROR2 mAb have high affinity and specificity for the human ROR2 and might be used to target ROR2 in various human cancers.

### Tables:

Table 1, shows biochemical properties of h6E6 mAb. Designations for the sixteen humanized ROR2 mAb are listed in the first column, mAb binding affinities (ka) for human ROR as measured by surface plasmon resonance are listed as nM concentrations. UNCLE thermostability measurements units are degrees Celsius (° C.). Tm₁ and Tm₂ refer to measurement in fluorescence over time related to mAb stability and Tagg is a measurement of thermal aggregation.

| | SPR for | | | |
|---|---|---|---|---|
| | Human | UNCLE Thermostability | | |
| | ROR2 | Measurements | | |
| mAb | k_{D} | Tm₁ | Tm₂ | T_{agg} |
| Name | (nM) | (° C.) | (° C.) | (° C.) |
| h6E6-007 | 0.318 | 68.6 | 81.2 | 65.2 |
| h6E6-029 | 0.079 | 69.3 | 80.6 | 66.2 |
| h6E6-040 | 0.186 | 72 | 81.3 | 63.3 |
| h6E6-055 | 0.261 | 69.7 | 78.5 | 68.1 |
| h6E6-056 | 0.024 | 70 | 80.3 | 65.4 |
| h6E6-057 | 0.463 | 69.4 | 82 | 71.4 |
| h6E6-058 | 0.187 | 68.6 | 83.3 | 74.2 |
| h6E6-063 | 0.475 | 69.5 | 83.2 | 68.9 |
| h6E6-066 | 0.353 | 70.2 | 81.6 | 71.2 |
| h6E6-068 | 0.362 | 69.6 | 77.9 | 73.9 |
| h6E6-070 | 0.165 | 69.2 | 79.2 | 65.6 |
| h6E6-072 | 0.380 | 69.4 | 79.4 | 65.4 |
| h6E6-077 | 0.155 | 69.1 | 82.9 | 73.3 |
| h6E6-102 | 0.319 | 69.5 | 81.2 | 68 |
| h6E6-107 | 0.422 | 68.8 | 80.7 | 76.8 |
| h6E6-116 | 0.348 | 69 | 81.1 | 70.4 |
| c6E6 | 0.168 | NT | NT | NT |

### Example 2: Humanization of Anti-ROR2 Antibodies

To humanize the murine 6E6 antibody, the heavy and light chain variable region protein sequences were aligned with human germline protein sequences to identify potential acceptor framework sequences. Based on this approach VK1-12*01, VK1-8*01 and VK3-15*01 were evaluated as acceptors for the light chain and VH3-21*01 and VH3-11*03 were evaluated as acceptors for the heavy chain. Next, the murine 6E6 CDRs (IMGT or extended) were grafted into the acceptor frameworks. The grafted CDRs are shown in Table 2. In certain cases, use of the extended CDR definition resulted in additional sequence differences between the murine donor CDR and the human acceptor germline framework.

TABLE 2
IMGT and extended CDR sequences.

| Chain | CDR | IMGT Sequence | Extended Sequence |
|---|---|---|---|
| Heavy | 1 | GFTFSNYG | GFTFSNYGMS |
| | 2 | ISSGGGYT | TISSGGGYTHYV |
| | 3 | ARHPRDFSYAMDY | ARHPRDFSYAMDY |
| Light | 1 | QDVGHY | KASQDVGHYVA |
| | 2 | WAS | WASTRHT |
| | 3 | QQYNIYPWT | QQYNIYPWT |

Using the different acceptor frameworks and CDR definitions, twenty-four humanized variants were expressed as IgG, purified and binding activity to human ROR2 was assessed by SPR. Multiple combinations were active binders while other combinations were inactive, as summarized in Table 3. Multiple variants displayed K_{D}s similar to chimeric 6E6 (K_{D}=0.38 nM). For example, extended CDRs grafted into both VK1-12*01 and VH3-21*01 (K_{D}=0.67 nM) and IMGT CDRs grafted into VK3-15*01 combined with extended CDRs grafted into VH3-21*01 (K_{D}=0.58 nM).

TABLE 3
Grafted humanized antibodies (K_{D} values in nM, N.B., no binding).

| | | Extended CDR | | IMGT CDR | |
|---|---|---|---|---|---|
| | | VH3-21*01 | VH3-11*03 | VH3-21*01 | VH3-11*03 |
| Extended | VK1-12*01 | 0.67 | 2.1 | N.B. | 162 |
| CDR | VK1-8*01 | 10 | 2.5 | N.B. | N.B. |
| | VK3-15*01 | 0.54 | 0.98 | N.B. | 98 |
| IMGT CDR | VK1-12*01 | 0.52 | N.B. | N.B. | 178 |
| | VK1-8*01 | 10 | 1.1 | N.B. | N.B. |
| | VK3-15*01 | 0.58 | 1.1 | N.B. | 64 |

### Example 3: Synthesis and Screening of Libraries to Remove Potential Post-Translational Modification Sites (PTMs)

Four potential translational modification (PTM) sites were identified in the variable heavy chain. These include an asparagine deamidation and methionine oxidation site in HCDR1, an aspartic acid isomerization site in framework 3 adjacent to HCDR2 and a methionine oxidation site in HCDR3. The PTMs and adjacent sequences are shown in Table 4 (PTM sequences in bold).

TABLE 4
Variable heavy chain PTMs.

| | | PTM and | |
|---|---|---|---|
| PTM | Potential | Adjacent | |
| Site | PTM | Sequence | Library |
| 1 | Asn | TFSNYGMSWV | A, D, E, F, G, H, I, K, |
| | deamidation | | L, N, P, Q, R, S, T, V, |
| | | | W, Y |
| 2 | Met | TFSNYGMSWV | A, E, F, G, H, I, K, L, |
| | oxidation | | M, P, Q, R, S, T, V, W, |
| | | | Y |
| 3 | Asp iso- | YTHYVDSVKG | A, D, E, G, H, P, Q, T, |
| | merization | | V |
| 4 | Met | DFSYAMDYWG | A, D, E, F, G, H, I, K, |
| | oxidation | | L, M, P, Q, R, S, T, V, |
| | | | W, Y |

To remove as many PTMs as possible, a combinatorial library was synthesized, expressed and screened. The library was designed to replace the PTM at each site with certain amino acids, as indicated in Table 4. In addition, the combinatorial library contained either the murine or the human residue at positions that differed between the murine donor and human acceptor sequences in the extended CDR definition, as shown in Table 5 (differences indicated in bold).

TABLE 5
Murine versus human residue at non-IMGT
positions within the extended CDR.

| | | Murine | Library |
|---|---|---|---|
| Chain | Site | Amino Acid(s) | (murine/human) |
| Heavy | CDR1 | GFTFSNYGMS | S/N |
| | CDR2 | TISSGGGYTHYV | T/S, H/Y, V/A |
| Light | CDR1 | KASQDVGHYVA | K/R, V/L |
| | CDR2 | WASTRHT | H/A |

The combinatorial library was synthesized and expressed as scFv using a phagemid system. The most active binders were enriched with four rounds of selection against human ROR2, using decreasing concentrations of human ROR2 for each round of enrichment.

Following the last round of selection enriched clones were screened by ELISA for binding to human ROR2. One hundred twenty-five clones displaying greater binding than the parental antibody were sequenced, yielding 120 unique VH/VL sequences (SEQ ID NO:65-SEQ ID NO:304). The screening results are summarized in Tables 6-8.

Alternative amino acids were identified for all four PTMs. For example, a number of suitable mutations at PTM site 1 were identified, with serine being the most common replacement (Table 6).

TABLE 6
PTM sites 1 and 2 screening results.

| PTM Site 1: TFSNYGMSWV | | | PTM Site 2: TFSNYGMSWV | | |
|---|---|---|---|---|---|
| | | Percent | | | Percent |
| Residue | Counts | of Total | Residue | Counts | of Total |
| S | 19 | 16% | M | 44 | 37% |
| G | 16 | 13% | V | 17 | 14% |
| N | 14 | 12% | T | 13 | 11% |
| K | 13 | 11% | I | 12 | 10% |
| A | 12 | 10% | L | 12 | 10% |
| H | 12 | 10% | H | 9 | 8% |
| R | 10 | 8% | A | 6 | 5% |
| T | 9 | 8% | Q | 4 | 3% |
| Q | 7 | 6% | S | 3 | 3% |
| V | 3 | 3% | | | |
| P | 2 | 2% | | | |
| D | 1 | 1% | | | |
| E | 1 | 1% | | | |
| F | 1 | 1% | | | |

Likewise, multiple active substitutions were identified at PTM site 2 with valine being the most common (Table 6), PTM site 3 with glutamate being the most common (Table 7), and PTM site 4 with leucine being the most common (Table 7). Notably, there were multiple suitable substitutions identified at each of the four variable heavy chain PTM sites.

TABLE 7
PTM sites 3 and 4 screening results.

| PTM Site 3: YTHYVDSVKG | | | PTM Site 4: DFSYAMDYWG | | |
|---|---|---|---|---|---|
| | | Percent | | | Percent |
| Residue | Counts | of Total | Residue | Counts | of Total |
| E | 29 | 24% | M | 41 | 34% |
| P | 26 | 22% | L | 24 | 20% |
| D | 18 | 15% | Q | 20 | 17% |
| A | 15 | 13% | H | 10 | 8% |
| T | 15 | 13% | F | 8 | 7% |
| Q | 9 | 8% | I | 5 | 4% |
| H | 5 | 4% | N | 4 | 3% |
| G | 3 | 3% | V | 3 | 3% |
| | | | S | 2 | 2% |
| | | | A | 1 | 1% |
| | | | K | 1 | 1% |
| | | | T | 1 | 1% |

### Example 4: Characterization of Enriched Clones

Sixty-four clones were reformatted as IgG, expressed and purified. The binding of the clones to human ROR2 was characterized by SPR (summarized in Table 8).

TABLE 8
Affinities of humanized and optimized murine 6E6 antibody.

| Clone | KD (nM) |
|---|---|
| 2 | 0.284 |
| 3 | 0.321 |
| 5 | 0.476 |
| 6 | 0.166 |
| 7 | 0.318 |
| 9 | 0.215 |
| 10 | 0.230 |
| 12 | 1.400 |
| 14 | 0.057 |
| 15 | 0.386 |
| 16 | 0.037 |
| 17 | 4.000 |
| 20 | 0.629 |
| 22 | 0.271 |
| 23 | 0.200 |
| 24 | 0.605 |
| 25 | 0.509 |
| 26 | 0.335 |
| 29 | 0.079 |
| 30 | 0.221 |
| 31 | 0.103 |
| 32 | 0.479 |
| 33 | 0.144 |
| 38 | 0.147 |
| 40 | 0.186 |
| 45 | 0.134 |
| 46 | 0.527 |
| 47 | 0.086 |
| 49 | 1.400 |
| 50 | 0.482 |
| 51 | 0.160 |
| 52 | 0.533 |
| 54 | 0.178 |
| 55 | 0.261 |
| 56 | 0.024 |
| 57 | 0.463 |
| 58 | 0.187 |
| 62 | 0.839 |
| 63 | 0.475 |
| 64 | 5.800 |
| 66 | 0.353 |
| 68 | 0.362 |
| 69 | 0.407 |
| 70 | 0.165 |
| 71 | 0.582 |
| 72 | 0.380 |
| 76 | 0.203 |
| 77 | 0.155 |
| 78 | 0.476 |
| 81 | 0.062 |
| 82 | 0.812 |
| 84 | 0.108 |
| 89 | 2.200 |
| 90 | 0.178 |
| 97 | 0.096 |
| 99 | 0.286 |
| 102 | 0.319 |
| 106 | 0.642 |
| 107 | 0.422 |
| 109 | 0.151 |
| 110 | 0.073 |
| 115 | 0.187 |
| 116 | 0.348 |
| 117 | 0.323 |
| Ch6E6 | 0.168 |

The majority of clones (58%) displayed affinities within two-fold (K_{D}<0.340 nM) of the chimeric parental 6E6 antibody. Furthermore, twenty-one of the clones had all of the targeted sequence liabilities (PTMs) removed. Sixteen clones with the targeted sequence liabilities removed were selected for larger-scale expression. All sixteen clones expressed well, with protein yields ranging from 400-1920 micrograms from 8 mL cultures. Following purification the purity of the clones was demonstrated by SDS capillary electrophoresis sodium dodecyl sulfate (CE-SDS). Additionally, the monomeric content of the antibodies was assessed by analytical SEC and the thermostability of the antibodies was characterized using the Uncle instrument (Unchained Laboratories). The results of these characterization studies are summarized in Table 9.

TABLE 9
Characterization of humanized 6E6 variants.

| | Uncle Thermostability | | | | |
|---|---|---|---|---|---|
| | Measurements | | | 8 mL | aSEC |
| | Tm₁ | Tm₂ | Tagg | Expression | % |
| Clone | (° C.) | (° C.) | (° C.) | (mg) | monomer |
| 7 | 68.6 | 81.2 | 65.2 | 1390 | 82.8 |
| 29 | 69.3 | 80.6 | 66.2 | 833 | 91.0 |
| 40 | 72.0 | 81.3 | 63.3 | 1020 | 85.4 |
| 55 | 69.7 | 78.5 | 68.1 | 1240 | 89.0 |
| 56 | 70.0 | 80.3 | 65.4 | 411 | 78.9 |
| 57 | 69.4 | 82.0 | 71.4 | 444 | 86.4 |
| 58 | 68.6 | 83.3 | 74.2 | 877 | 91.0 |
| 63 | 69.5 | 83.2 | 68.9 | 948 | 90.6 |
| 66 | 70.2 | 81.6 | 71.2 | 833 | 90.3 |
| 68 | 69.6 | 77.9 | 73.9 | 1850 | 96.5 |
| 70 | 69.2 | 79.2 | 65.6 | 1050 | 93.3 |
| 72 | 69.4 | 79.4 | 65.4 | 790 | 77.8 |
| 77 | 69.1 | 82.9 | 73.3 | 427 | 58.7 |
| 102 | 69.5 | 81.2 | 68.0 | 1160 | 90.4 |
| 107 | 68.8 | 80.7 | 76.8 | 1620 | 95.7 |
| 116 | 69.0 | 81.1 | 70.4 | 1920 | 97.8 |

INFORMAL SEQUENCE LISTING

SEQ ID NO: 1, h6E6-007 VH SEQ
EVQLVESGGGLVKPGGSLRLSCAASGFTFSGYGLSWVRQAPGKGLEWVSTISSGGGYT
HYAPSVKGRFTISRDNAKNSLYLQMNSLRAEDTAVYYCARHPRDFSYAQDYWGQGTT
VTVSS
SEQ ID NO: 2, h6E6-007 VK SEQ
EIVMTQSPATLSVSPGERATLSCKASQDVGHYVAWYQQKPGQAPRLLIYWASTRATGIP
ARFSGSGSGTEFTLTISSLQSEDFAVYYCQQYNIYPWTFGQGTKVEIK
SEQ ID NO: 3, h6E6-029 VH SEQ
EVQLVESGGGLVKPGGSLRLSCAASGFTFSHYGVSWVRQAPGKGLEWVSTISSGGGYT
HYAQSVKGRFTISRDNAKNSLYLQMNSLRAEDTAVYYCARHPRDFSYAQDYWGQGTT
VTVSS
SEQ ID NO: 4, h6E6-029 VK SEQ
EIVMTQSPATLSVSPGERATLSCKASQDVGHYVAWYQQKPGQAPRLLIYWASTRHTGIP
ARFSGSGSGTEFTLTISSLQSEDFAVYYCQQYNIYPWTFGQGTKVEIK
SEQ ID NO: 5, h6E6-040 VH SEQ
EVQLVESGGGLVKPGGSLRLSCAASGFTFSSYGHSWVRQAPGKGLEWVSTISSGGGYTH
YAPSVKGRFTISRDNAKNSLYLQMNSLRAEDTAVYYCARHPRDFSYAHDYWGQGTTV
TVSS
SEQ ID NO: 6, h6E6-040 VK SEQ
EIVMTQSPATLSVSPGERATLSCRASQDVGHYVAWYQQKPGQAPRLLIYWASTRATGIP
ARFSGSGSGTEFTLTISSLQSEDFAVYYCQQYNIYPWTFGQGTKVEIK
SEQ ID NO: 7, h6E6-055 VH SEQ
EVQLVESGGGLVKPGGSLRLSCAASGFTFSQYGHSWVRQAPGKGLEWVSTISSGGGYT
HYAESVKGRFTISRDNAKNSLYLQMNSLRAEDTAVYYCARHPRDFSYAQDYWGQGTT
VTVSS
SEQ ID NO: 8, h6E6-055 VK SEQ
EIVMTQSPATLSVSPGERATLSCKASQDVGHYLAWYQQKPGQAPRLLIYWASTRATGIP
ARFSGSGSGTEFTLTISSLQSEDFAVYYCQQYNIYPWTFGQGTKVEIK
SEQ ID NO: 9, h6E6-056 VH SEQ
EVQLVESGGGLVKPGGSLRLSCAASGFTFSKYGTSWVRQAPGKGLEWVSTISSGGGYT
HYAPSVKGRFTISRDNAKNSLYLQMNSLRAEDTAVYYCARHPRDFSYAQDYWGQGTT
VTVSS
SEQ ID NO: 10, h6E6-056 VK SEQ
EIVMTQSPATLSVSPGERATLSCRASQDVGHYVAWYQQKPGQAPRLLIYWASTRATGIP
ARFSGSGSGTEFTLTISSLQSEDFAVYYCQQYNIYPWTFGQGTKVEIK
SEQ ID NO: 11, h6E6-057 VH SEQ
EVQLVESGGGLVKPGGSLRLSCAASGFTFSHYGVSWVRQAPGKGLEWVSTISSGGGYT
HYVASVKGRFTISRDNAKNSLYLQMNSLRAEDTAVYYCARHPRDFSYALDYWGQGTT
VTVSS
SEQ ID NO: 12, h6E6-057 VK SEQ
EIVMTQSPATLSVSPGERATLSCKASQDVGHYVAWYQQKPGQAPRLLIYWASTRATGIP
ARFSGSGSGTEFTLTISSLQSEDFAVYYCQQYNIYPWAFGQGTKVEIK
SEQ ID NO: 13, h6E6-058 VH SEQ
EVQLVESGGGLVKPGGSLRLSCAASGFTFSVYGLSWVRQAPGKGLEWVSTISSGGGYT
QYAPSVKGRFTISRDNAKNSLYLQMNSLRAEDTAVYYCARHPRDFSYAFDYWGQGTT
VTVSS
SEQ ID NO: 14, h6E6-058 VK SEQ
EIVMTQSPATLSVSPGERATLSCKASQDVGHYLAWYQQKPGQAPRLLIYWASTRATGIP
ARFSGSGSGTEFTLTISSLQSEDFAVYYCQQYNIYPWTFGQGTKVEIK
SEQ ID NO: 15, h6E6-063 VH SEQ
EVQLVESGGGLVKPGGSLRLSCAASGFTFSAYGTSWVRQAPGKGLEWVSTISSGGGYT
YYAASVKGRFTISRDNAKNSLYLQMNSLRAEDTAVYYCARHPRDFSYAIDYWGQGTTV
TVSS
SEQ ID NO: 16, h6E6-063 VK SEQ
EIVMTQSPATLSVSPGERATLSCRASQDVGHYLAWYQQKPGQAPRLLIYWASTRATGIP
ARFSGSGSGTEFTLTISSLQSEDFAVYYCQQYNIYPWTFGQGTKVEIK
SEQ ID NO: 17, h6E6-066 VH SEQ
EVQLVESGGGLVKPGGSLRLSCAASGFTFSAYGSSWVRQAPGKGLEWVSTISSGGGYTH
YAPSVKGRFTISRDNAKNSLYLQMNSLRAEDTAVYYCARHPRDFSYAHDYWGQGTTV
TVSS
SEQ ID NO: 18, h6E6-066 VK SEQ
EIVMTQSPATLSVSPGERATLSCKASQDVGHYVAWYQQKPGQAPRLLIYWASTRHTGIP
ARFSGSGSGTEFTLTISSLQSEDFAVYYCQQYNIYPWTFGQGTKVEIK
SEQ ID NO: 19, h6E6-068 VH SEQ
EVQLVESGGGLVKPGGSLRLSCAASGFTFSSYGISWVRQAPGKGLEWVSTISSGGGYTH
YAHSVKGRFTISRDNAKNSLYLQMNSLRAEDTAVYYCARHPRDFSYALDYWGQGTTV
TVSS
SEQ ID NO: 20, h6E6-068 VK SEQ
EIVMTQSPATLSVSPGERATLSCKASQDVGHYLAWYQQKPGQAPRLLIYWASTRATGIP
ARFSGSGSGTEFTLTISSLQSEDFAVYYCQQYNIYPWTFGQGTKVEIK
SEQ ID NO: 21, h6E6-070 VH SEQ
EVQLVESGGGLVKPGGSLRLSCAASGFTFSQYGHSWVRQAPGKGLEWVSTISSGGGYT
HYAHSVKGRFTISRDNAKNSLYLQMNSLRAEDTAVYYCARHPRDFSYANDYWGQGTT
VTVSS
SEQ ID NO: 22, h6E6-070 VK SEQ
EIVMTQSPATLSVSPGERATLSCRASQDVGHYLAWYQQKPGQAPRLLIYWASTRATGIP
ARFSGSGSGTEFTLTISSLQSEDFAVYYCQQYNIYPWTFGQGTKVEIK
SEQ ID NO: 23, h6E6-072 VH SEQ
EVQLVESGGGLVKPGGSLRLSCAASGFTFSQYGHSWVRQAPGKGLEWVSTISSGGGYT
HYADSVKGRFTISRDNAKNSLYLQMNSLRAEDTAVYYCARHPRDFSYAQDYWGQGTT
VTVSS
SEQ ID NO: 24, h6E6-072 VK SEQ
EIVMTQSPATLSVSPGERATLSCKASQDVGHYLAWYQQKPGQAPRLLIYWASTRHTGIP
ARFSGSGSGTEFTLTISSLQSEDFAVYYCQQYNIYPWTFGQGTKVEIK
SEQ ID NO:25, h6E6-077 VH SEQ
EVQLVESGGGLVKPGGSLRLSCAASGFTFSHYGTSWVRQAPGKGLEWVSTISSGGGYT
HYAPSVKGRFTISRDNAKNSLYLQMNSLRAEDTAVYYCARHPRDFSYAIDYWGQGTTV
TVSS
SEQ ID NO: 26, h6E6-077 VK SEQ
EIVMTQSPATLSVSPGERATLSCKASQDVGHYVAWYQQKPGQAPRLLIYWASTRATGIP
ARFSGSGSGTEFTLTISSLQSEDFAVYYCQQYNIYPWTFGQGTKVEIK
SEQ ID NO: 27, h6E6-102 VH SEQ
EVQLVESGGGLVKPGGSLRLSCAASGFTFSKYGLSWVRQAPGKGLEWVSTISSGGGYT
HYVESVKGRFTISRDNAKNSLYLQMNSLRAEDTAVYYCARHPRDFSYASDYWGQGTT
VTVSS
SEQ ID NO: 28, h6E6-102 VK SEQ
EIVMTQSPATLSVSPGERATLSCKASQDVGHYVAWYQQKPGQAPRLLIYWASTRATGIP
ARFSGSGSGTEFTLTISSLQSEDFAVYYCQQYNIYPWTFGQGTKVEIK
SEQ ID NO: 29, h6E6-107 VH SEQ
EVQLVESGGGLVKPGGSLRLSCAASGFTFSTYGASWVRQAPGKGLEWVSTISSGGGYT
HYAPSVKGRFTISRDNAKNSLYLQMNSLRAEDTAVYYCARHPRDFSYALDYWGQGTT
VTVSS
SEQ ID NO: 30, h6E6-107 VK SEQ
EIVMTQSPATLSVSPGERATLSCKASQDVGHYVAWYQQKPGQAPRLLIYWASTRATGIP
ARFSGSGSGTEFTLTISSLQSEDFAVYYCQQYNIYPWTFGQGTKVEIK
SEQ ID NO: 31, h6E6-116 VH SEQ
EVQLVESGGGLVKPGGSLRLSCAASGFTFSTYGVSWVRQAPGKGLEWVSTISSGGGYT
HYAGSVKGRFTISRDNAKNSLYLQMNSLRAEDTAVYYCARHPRDFSYALDYWGQGTT
VTVSS
SEQ ID NO: 32, h6E6-116 VK SEQ
EIVMTQSPATLSVSPGERATLSCRASQDVGHYLAWYQQKPGQAPRLLIYWASTRATGIP
ARFSGSGSGTEFTLTISSLQSEDFAVYYCQQYNIYPWTFGQGTKVEIK
SEQ ID NO: 33, HUMAN ROR2 FULL LENGTH
ATGGCCCGGGGCTCGGCGCTCCCGCGGCGGCCGCTGCTGTGCATCCCGGCCGTCTGG
GCGGCCGCCGCGCTTCTGCTCTCAGTGTCCCGGACTTCAGGTGAAGTGGAGGTTCTG
GATCCGAACGACCCTTTAGGACCCCTTGATGGGCAGGACGGCCCGATTCCAACTCTG
AAAGGTTACTTTCTGAATTTTCTGGAGCCAGTAAACAATATCACCATTGTCCAAGGC
CAGACGGCAATTCTGCACTGCAAGGTGGCAGGAAACCCACCCCCTAACGTGCGGTG
GCTAAAGAATGATGCCCCGGTGGTGCAGGAGCCGCGGCGGATCATCATCCGGAAGA
CAGAATATGGTTCACGACTGCGAATCCAGGACCTGGACACGACAGACACTGGCTAC
TACCAGTGCGTGGCCACCAACGGGATGAAGACCATTACCGCCACTGGCGTCCTGTTT
GTGCGGCTGGGTCCAACGCACAGCCCAAATCATAACTTTCAGGATGATTACCACGA
GGATGGGTTCTGCCAGCCTTACCGGGGAATTGCCTGTGCACGCTTCATTGGCAACCG
GACCATTTATGTGGACTCGCTTCAGATGCAGGGGGAGATTGAAAACCGAATCACAG
CGGCCTTCACCATGATCGGCACGTCTACGCACCTGTCGGACCAGTGCTCACAGTTCG
CCATCCCATCCTTCTGCCACTTCGTGTTTCCTCTGTGCGACGCGCGCTCCCGGGCACC
CAAGCCGCGTGAGCTGTGCCGCGACGAGTGCGAGGTGCTGGAGAGCGACCTGTGCC
GCCAGGAGTACACCATCGCCCGCTCCAACCCGCTCATCCTCATGCGGCTTCAGCTGC
CCAAGTGTGAGGCGCTGCCCATGCCTGAGAGCCCCGACGCTGCCAACTGCATGCGC
ATTGGCATCCCAGCCGAGAGGCTGGGCCGCTACCATCAGTGCTATAACGGCTCAGG
CATGGATTACAGAGGAACGGCAAGCACCACCAAGTCAGGCCACCAGTGCCAGCCGT
GGGCCCTGCAGCACCCCCACAGCCACCACCTGTCCAGCACAGACTTCCCTGAGCTTG
GAGGGGGGCACGCCTACTGCCGGAACCCCGGAGGCCAGATGGAGGGCCCCTGGTGC
TTTACGCAGAATAAAAACGTACGCATGGAACTGTGTGACGTACCCTCGTGTAGTCCC
CGAGACAGCAGCAAGATGGGGATTCTGTACATCTTGGTCCCCAGCATCGCAATTCCA
CTGGTCATCGCTTGCCTTTTCTTCTTGGTTTGCATGTGCCGGAATAAGCAGAAGGCAT
CTGCGTCCACACCGCAGCGGCGACAGCTGATGGCCTCGCCCAGCCAAGACATGGAA
ATGCCCCTCATTAACCAGCACAAACAGGCCAAACTCAAAGAGATCAGCCTGTCTGC
GGTGAGGTTCATGGAGGAGCTGGGAGAGGACCGGTTTGGGAAAGTCTACAAAGGTC
ACCTGTTCGGCCCTGCCCCGGGGGAGCAGACCCAGGCTGTGGCCATCAAAACGCTG
AAGGACAAAGCGGAGGGGCCCCTGCGGGAGGAGTTCCGGCATGAGGCTATGCTGCG
AGCACGGCTGCAACACCCCAACGTCGTCTGCCTGCTGGGCGTGGTGACCAAGGACC
AGCCCCTGAGCATGATCTTCAGCTACTGTTCGCACGGCGACCTCCACGAATTCCTGG
TCATGCGCTCGCCGCACTCGGACGTGGGCAGCACCGATGATGACCGCACGGTGAAG
TCCGCCCTGGAGCCCCCCGACTTCGTGCACCTTGTGGCACAGATCGCGGCGGGGATG
GAGTACCTATCCAGCCACCACGTGGTTCACAAGGACCTGGCCACCCGCAATGTGCTA
GTGTACGACAAGCTGAACGTGAAGATCTCAGACTTGGGCCTCTTCCGAGAGGTGTAT
GCCGCCGATTACTACAAGCTGCTGGGGAACTCGCTGCTGCCTATCCGCTGGATGGCC
CCAGAGGCCATCATGTACGGCAAGTTCTCCATCGACTCAGACATCTGGTCCTACGGT
GTGGTCCTGTGGGAGGTCTTCAGCTACGGCCTGCAGCCCTACTGCGGGTACTCCAAC
CAGGATGTGGTGGAGATGATCCGGAACCGGCAGGTGCTGCCTTGCCCCGATGACTG
TCCCGCCTGGGTGTATGCCCTCATGATCGAGTGCTGGAACGAGTTCCCCAGCCGGCG
GCCCCGCTTCAAGGACATCCACAGCCGGCTCCGAGCCTGGGGCAACCTTTCCAACTA
CAACAGCTCGGCGCAGACCTCGGGGGCCAGCAACACCACGCAGACCAGCTCCCTGA
GCACCAGCCCAGTGAGCAATGTGAGCAACGCCCGCTACGTGGGGCCCAAGCAGAAG
GCCCCGCCCTTCCCACAGCCCCAGTTCATCCCCATGAAGGGCCAGATCAGACCCATG
GTGCCCCCGCCGCAGCTCTACGTCCCCGTCAACGGCTACCAGCCGGTGCCGGCCTAT
GGGGCCTACCTGCCCAACTTCTACCCGGTGCAGATCCCAATGCAGATGGCCCCGCAG
CAGGTGCCTCCTCAGATGGTCCCCAAGCCCAGCTCACACCACAGTGGCAGTGGCTCC
ACCAGCACAGGCTACGTCACCACGGCCCCCTCCAACACATCCATGGCAGACAGGGC
AGCCCTGCTCTCAGAGGGCGCTGATGACACACAGAACGCCCCAGAAGATGGGGCCC
AGAGCACCGTGCAGGAAGCAGAGGAGGAGGAGGAAGGCTCTGTCCCAGAGACTGA
GCTGCTGGGGGACTGTGACACTCTGCAGGTGGACGAGGCCCAAGTCCAGCTGGAAG
CTTGA
SEQ ID NO: 34, HUMAN ROR2 FULL LENGTH
MARGSALPRRPLLCIPAVWAAAALLLSVSRTSGEVEVLDPNDPLGPLDGQDGPIPTLKG
YFLNFLEPVNNITIVQGQTAILHCKVAGNPPPNVRWLKNDAPVVQEPRRIIIRKTEYGSRL
RIQDLDTTDTGYYQCVATNGMKTITATGVLFVRLGPTHSPNHNFQDDYHEDGFCQPYR
GIACARFIGNRTIYVDSLQMQGEIENRITAAFTMIGTSTHLSDQCSQFAIPSFCHFVFPLCD
ARSRTPKPRELCRDECEVLESDLCRQEYTIARSNPLILMRLQLPKCEALPMPESPDAANC
MRIGIPAERLGRYHQCYNGSGMDYRGTASTTKSGHQCQPWALQHPHSHHLSSTDFPEL
GGGHAYCRNPGGQMEGPWCFTQNKNVRMELCDVPSCSPRDSSKMGILYILVPSIAIPLVI
ACLFFLVCMCRNKQKASASTPQRRQLMASPSQDMEMPLINQHKQAKLKEISLSAVRFM
EELGEDRFGKVYKGHLFGPAPGEQTQAVAIKTLKDKAEGPLREEFRHEAMLRARLQHP
NVVCLLGVVTKDQPLSMIFSYCSHGDLHEFLVMRSPHSDVGSTDDDRTVKSALEPPDFV
HLVAQIAAGMEYLSSHHVVHKDLATRNVLVYDKLNVKISDLGLFREVYAADYYKLLG
NSLLPIRWMAPEAIMYGKFSIDSDIWSYGVVLWEVFSYGLQPYCGYSNQDVVEMIRNR
QVLPCPDDCPAWVYALMIECWNEFPSRRPRFKDIHSRLRAWGNLSNYNSSAQTSGASN
TTQTSSLSTSPVSNVSNARYVGPKQKAPPFPQPQFIPMKGQIRPMVPPPQLYVPVNGYQP
VPAYGAYLPNFYPVQIPMQMAPQQVPPQMVPKPSSHHSGSGSTSTGYVTTAPSNTSMA
DRAALLSEGADDTQNAPEDGAQSTVQEAEEEEEGSVPETELLGDCDTLQVDEAQVQLEA
SEQ ID NO: 35, MOUSE ROR2 FULL LENGTH
ATGGCTCGGGGCTGGGTGCGGCCGAGCCGTGTGCCTCTGTGCGCCCGGGCCGTCTGG
ACGGCTGCGGCGCTCCTGCTCTGGACACCCTGGACGGCAGGTGAAGTGGAAGATTC
GGAGGCAATCGACACCTTGGGACAACCTGATGGACCGGACAGCCCACTTCCCACTC
TGAAAGGCTACTTTCTGAATTTTCTGGAGCCAGTCAACAATATCACCATTGTTCAGG
GCCAGACGGCAATCCTGCACTGCAAGGTGGCGGGAAACCCACCTCCCAATGTGCGG
TGGCTGAAGAATGATGCCCCGGTTGTGCAAGAGCCACGAAGGGTCGTCATCCGGAA
GACAGAATACGGCTCCCGGCTGCGGATCCAAGACCTGGACACAACAGACACAGGCT
ACTACCAGTGTGTGGCTACCAACGGGCTGAAGACCATCACTGCCACTGGGGTTCTAT
ATGTGCGGCTCGGTCCGACGCACAGCCCGAACCACAATTTTCAGGATGACGATCAG
GAAGATGGCTTCTGCCAGCCGTACCGAGGGATCGCTTGTGCGCGCTTCATTGGGAAC
CGGACTATTTATGTGGACTCCCTCCAGATGCAGGGGGAGATTGAAAACCGAATCAC
AGCTGCCTTCACCATGATCGGCACCTCCACGCAACTGTCAGACCAGTGTTCACAGTT
TGCCATCCCATCCTTCTGCCACTTCGTCTTCCCTCTGTGCGACGCATGCTCCCGGGCG
CCCAAGCCTCGCGAACTGTGCCGGGATGAATGTGAGGTGCTGGAGAACGACCTGTG
CCGCCAGGAGTACACCATCGCCCGCTCCAACCCGCTCATCCTCATGCGGCTCCAGCT
GCCCAAGTGCGAAGCGCTGCCCATGCCCGAGAGCCCGGATGCTGCGAACTGCATGC
GCATCGGGATCCCCGCGGAGAGGCTGGGTCGCTACCACCAGTGCTACAACGGCTCC
GGCGCCGATTACAGGGGGATGGCCAGTACCACCAAGTCAGGCCACCAGTGTCAGCC
TTGGGCTCTGCAGCACCCCCACAGCCATCGCCTATCCAGCACGGAATTCCCTGAGCT
GGGAGGAGGCCATGCCTACTGCCGGAACCCCGGGGGCCAGATGGAAGGCCCGTGGT
GCTTTACGCAGAATAAAAACGTACGCGTGGAACTGTGTGACGTACCCCCGTGTAGTC
CCCGATATGGCAGCAAGATGGGGATTCTGTACATCCTGGTCCCCAGCATTGCTATCC
CCCTGGTCATCGCTTGCCTGTTCTTCCTCGTCTGCATGTGCCGCAACAAACAGAAGG
CTTCGGCCTCCACCCCACAGCGCCGGCAGCTGATGGCCTCTCCCAGCCAGGACATGG
AGATGCCACTCATCAGCCAGCACAAACAGGCCAAACTCAAAGAGATCAGCTTGTCC
ACAGTGAGGTTCATGGAGGAGCTCGGGGAGGACCGGTTTGGCAAGGTCTACAAAGG
CCACCTGTTCGGGCCTGCCCCAGGAGAACCAACCCAGGCCGTGGCCATCAAGACGC
TGAAAGACAAGGCTGAGGGGCCCCTGCGGGAGGAGTTCCGGCAAGAGGCGATGCTC
CGGGCCCGACTGCAGCACCCCAACATCGTCTGTCTCCTAGGCGTCGTGACCAAGGAC
CAACCCTTGAGCATGATCTTCAGCTACTGTTCCCATGGCGACCTTCATGAATTCCTG
GTCATGCGCTCGCCGCACTCCGATGTGGGCAGCACCGATGACGACCGCACAGTGAA
GTCAGCCCTGGAGCCCCCGGACTTCGTGCACGTGGTGGCGCAGATCGCTGCGGGGA
TGGAGTTCCTGTCCAGCCACCACGTGTGCCATAAGGACCTGGCCACACGCAATGTGC
TGGTGTACGACAAGCTGAACGTGAGGATCTCAGACTTGGGCCTCTTCCGTGAGGTAT
ACTCCGCAGATTACTACAAACTCATGGGCAATTCACTGCTGCCCATCCGCTGGATGT
CCCCCGAGGCCGTCATGTATGGAAAGTTCTCCATCGACTCTGACATCTGGTCCTACG
GTGTGGTCCTCTGGGAGGTCTTTAGCTACGGCCTGCAGCCCTACTGTGGGTACTCCA
ACCAGGACGTGGTGGAGATGATCCGGAGCCGGCAGGTGCTGCCCTGCCCGGATGAC
TGCCCCGCCTGGGTCTATGCCCTCATGATTGAATGCTGGAATGAGTTCCCAAGCCGG
AGGCCCCGCTTTAAGGACATCCACAGCCGGCTCCGGTCCTGGGGCAACCTATCCAAC
TATAATAGTTCCGCGCAGACCTCAGGAGCCAGCAACACCACACAGACCAGCTCCCT
GAGCACCAGCCCCGTAAGCAATGTGAGCAATGCCCGCTATATGGCCCCCAAGCAGA
AGGCCCAGCCCTTCCCACAGCCTCAGTTCATCCCCATGAAGGGTCAGATCAGACCCT
TGGTGCCCCCCGCACAGCTGTACATCCCGGTGAACGGCTATCAGCCGGTACCGGCAT
ACGGGGCCTACCTGCCCAACTTCTACCCAGTCCAGATCCCCATGCAGATGGCCCCAC
AGCAGGTGCCCCCTCAGATGGTCCCCAAGCCGAGCTCACACCACAGTGGCAGCGGC
TCCACCAGCACTGGCTACGTCACCACGGCGCCCTCCAATACATCTGTGGCGGACAGG
GCGGCCCTACTCTCTGAGGGCACCGAGGATGTACAGAACATCGCGGAAGACGTGGC
CCAGAGCCCTGTGCAGGAAGCAGAGGAGGAGGAGGAGGGGTCTGTCCCTGAGACT
GAACTCCTGGGAGACAATGACACGCTCCAGGTGACCGAGGCGGCTCATGTCCAGCT
TGAAGCCTGA
SEQ ID NO: 36, MOUSE ROR2 FULL LENGTH
MARGWVRPSRVPLCARAVWTAAALLLWTPWTAGEVEDSEAIDTLGQPDGPDSPLPTLK
GYFLNFLEPVNNITIVQGQTAILHCKVAGNPPPNVRWLKNDAPVVQEPRRVVIRKTEYG
SRLRIQDLDTTDTGYYQCVATNGLKTITATGVLYVRLGPTHSPNHNFQDDDQEDGFCQP
YRGIACARFIGNRTIYVDSLQMQGEIENRITAAFTMIGTSTQLSDQCSQFAIPSFCHFVFPL
CDACSRAPKPRELCRDECEVLENDLCRQEYTIARSNPLILMRLQLPKCEALPMPESPDAA
NCMRIGIPAERLGRYHQCYNGSGADYRGMASTTKSGHQCQPWALQHPHSHRLSSTEFP
ELGGGHAYCRNPGGQMEGPWCFTQNKNVRVELCDVPPCSPRYGSKMGILYILVPSIAIP
LVIACLFFLVCMCRNKQKASASTPQRRQLMASPSQDMEMPLISQHKQAKLKEISLSTVR
FMEELGEDRFGKVYKGHLFGPAPGEPTQAVAIKTLKDKAEGPLREEFRQEAMLRARLQ
HPNIVCLLGVVTKDQPLSMIFSYCSHGDLHEFLVMRSPHSDVGSTDDDRTVKSALEPPDF
VHVVAQIAAGMEFLSSHHVCHKDLATRNVLVYDKLNVRISDLGLFREVYSADYYKLM
GNSLLPIRWMSPEAVMYGKFSIDSDIWSYGVVLWEVFSYGLQPYCGYSNQDVVEMIRS
RQVLPCPDDCPAWVYALMIECWNEFPSRRPRFKDIHSRLRSWGNLSNYNSSAQTSGASN
TTQTSSLSTSPVSNVSNARYMAPKQKAQPFPQPQFIPMKGQIRPLVPPAQLYIPVNGYQP
VPAYGAYLPNFYPVQIPMQMAPQQVPPQMVPKPSSHHSGSGSTSTGYVTTAPSNTSVAD
RAALLSEGTEDVQNIAEDVAQSPVQEAEEEEEGSVPETELLGDNDTLQVTEAAHVQLEA
SEQ ID NO: 37, HUMAN ROR2 Extracellular Region
ATGGCCCGGGGCTCGGCGCTCCCGCGGCGGCCGCTGCTGTGCATCCCGGCCGTCTGG
GCGGCCGCCGCGCTTCTGCTCTCAGTGTCCCGGACTTCAGGTGAAGTGGAGGTTCTG
GATCCGAACGACCCTTTAGGACCCCTTGATGGGCAGGACGGCCCGATTCCAACTCTG
AAAGGTTACTTTCTGAATTTTCTGGAGCCAGTAAACAATATCACCATTGTCCAAGGC
CAGACGGCAATTCTGCACTGCAAGGTGGCAGGAAACCCACCCCCTAACGTGCGGTG
GCTAAAGAATGATGCCCCGGTGGTGCAGGAGCCGCGGCGGATCATCATCCGGAAGA
CAGAATATGGTTCACGACTGCGAATCCAGGACCTGGACACGACAGACACTGGCTAC
TACCAGTGCGTGGCCACCAACGGGATGAAGACCATTACCGCCACTGGCGTCCTGTTT
GTGCGGCTGGGTCCAACGCACAGCCCAAATCATAACTTTCAGGATGATTACCACGA
GGATGGGTTCTGCCAGCCTTACCGGGGAATTGCCTGTGCACGCTTCATTGGCAACCG
GACCATTTATGTGGACTCGCTTCAGATGCAGGGGGAGATTGAAAACCGAATCACAG
CGGCCTTCACCATGATCGGCACGTCTACGCACCTGTCGGACCAGTGCTCACAGTTCG
CCATCCCATCCTTCTGCCACTTCGTGTTTCCTCTGTGCGACGCGCGCTCCCGGGCACC
CAAGCCGCGTGAGCTGTGCCGCGACGAGTGCGAGGTGCTGGAGAGCGACCTGTGCC
GCCAGGAGTACACCATCGCCCGCTCCAACCCGCTCATCCTCATGCGGCTTCAGCTGC
CCAAGTGTGAGGCGCTGCCCATGCCTGAGAGCCCCGACGCTGCCAACTGCATGCGC
ATTGGCATCCCAGCCGAGAGGCTGGGCCGCTACCATCAGTGCTATAACGGCTCAGG
CATGGATTACAGAGGAACGGCAAGCACCACCAAGTCAGGCCACCAGTGCCAGCCGT
GGGCCCTGCAGCACCCCCACAGCCACCACCTGTCCAGCACAGACTTCCCTGAGCTTG
GAGGGGGGCACGCCTACTGCCGGAACCCCGGAGGCCAGATGGAGGGCCCCTGGTGC
TTTACGCAGAATAAAAACGTACGCATGGAACTGTGTGACGTACCCTCGTGTAGTCCC
CGAGACAGCAGCAAGATGGGG
SEQ ID NO: 38, HUMAN ROR2 Extracellular Region
MARGSALPRRPLLCIPAVWAAAALLLSVSRTSGEVEVLDPNDPLGPLDGQDGPIPTLKG
YFLNFLEPVNNITIVQGQTAILHCKVAGNPPPNVRWLKNDAPVVQEPRRIIIRKTEYGSRL
RIQDLDTTDTGYYQCVATNGMKTITATGVLFVRLGPTHSPNHNFQDDYHEDGFCQPYR
GIACARFIGNRTIYVDSLQMQGEIENRITAAFTMIGTSTHLSDQCSQFAIPSFCHFVFPLCD
ARSRTPKPRELCRDECEVLESDLCRQEYTIARSNPLILMRLQLPKCEALPMPESPDAANC
MRIGIP AERLGRYHQCYNGSGMDYRGTASTTKSGHQCQPWALQHPHSHHLSSTDFPEL
GGGHAYCRNPGGQMEGPWCFTQNKNVRMELCDVPSCSPRDSSKMG
SEQ ID NO: 39, MOUSE ROR2 Extracellular Region
ATGGCTCGGGGCTGGGTGCGGCCGAGCCGTGTGCCTCTGTGCGCCCGGGCCGTCTGG
ACGGCTGCGGCGCTCCTGCTCTGGACACCCTGGACGGCAGGTGAAGTGGAAGATTC
GGAGGCAATCGACACCTTGGGACAACCTGATGGACCGGACAGCCCACTTCCCACTC
TGAAAGGCTACTTTCTGAATTTTCTGGAGCCAGTCAACAATATCACCATTGTTCAGG
GCCAGACGGCAATCCTGCACTGCAAGGTGGCGGGAAACCCACCTCCCAATGTGCGG
TGGCTGAAGAATGATGCCCCGGTTGTGCAAGAGCCACGAAGGGTCGTCATCCGGAA
GACAGAATACGGCTCCCGGCTGCGGATCCAAGACCTGGACACAACAGACACAGGCT
ACTACCAGTGTGTGGCTACCAACGGGCTGAAGACCATCACTGCCACTGGGGTTCTAT
ATGTGCGGCTCGGTCCGACGCACAGCCCGAACCACAATTTTCAGGATGACGATCAG
GAAGATGGCTTCTGCCAGCCGTACCGAGGGATCGCTTGTGCGCGCTTCATTGGGAAC
CGGACTATTTATGTGGACTCCCTCCAGATGCAGGGGGAGATTGAAAACCGAATCAC
AGCTGCCTTCACCATGATCGGCACCTCCACGCAACTGTCAGACCAGTGTTCACAGTT
TGCCATCCCATCCTTCTGCCACTTCGTCTTCCCTCTGTGCGACGCATGCTCCCGGGCG
CCCAAGCCTCGCGAACTGTGCCGGGATGAATGTGAGGTGCTGGAGAACGACCTGTG
CCGCCAGGAGTACACCATCGCCCGCTCCAACCCGCTCATCCTCATGCGGCTCCAGCT
GCCCAAGTGCGAAGCGCTGCCCATGCCCGAGAGCCCGGATGCTGCGAACTGCATGC
GCATCGGGATCCCCGCGGAGAGGCTGGGTCGCTACCACCAGTGCTACAACGGCTCC
GGCGCCGATTACAGGGGGATGGCCAGTACCACCAAGTCAGGCCACCAGTGTCAGCC
TTGGGCTCTGCAGCACCCCCACAGCCATCGCCTATCCAGCACGGAATTCCCTGAGCT
GGGAGGAGGCCATGCCTACTGCCGGAACCCCGGGGGCCAGATGGAAGGCCCGTGGT
GCTTTACGCAGAATAAAAACGTACGCGTGGAACTGTGTGACGTACCCCCGTGTAGTC
CCCGATATGGCAGCAAGATGGGG
SEQ ID NO: 40, MOUSE ROR2 Extracellular Region
MARGWVRPSRVPLCARAVWTAAALLLWTPWTAGEVEDSEAIDTLGQPDGPDSPLPTLK
GYFLNFLEPVNNITIVQGQTAILHCKVAGNPPPNVRWLKNDAPVVQEPRRVVIRKTEYG
SRLRIQDLDTTDTGYYQCVATNGLKTITATGVLYVRLGPTHSPNHNFQDDDQEDGFCQP
YRGIACARFIGNRTIYVDSLQMQGEIENRITAAFTMIGTSTQLSDQCSQFAIPSFCHFVFPL
CDACSRAPKPRELCRDECEVLENDLCRQEYTIARSNPLILMRLQLPKCEALPMPESPDAA
NCMRIGIPAERLGRYHQCYNGSGADYRGMASTTKSGHQCQPWALQHPHSHRLSSTEFP
ELGGGHAYCRNPGGQMEGPWCFTQNKNVRVELCDVPPCSPRYGSKMG
SEQ ID NO: 41; Human ROR2 Kringle domain
HQCYNGSGMDYRGTASTTKSGHQCQPWALQHPHSHHLSSTDFPELGGGHAYCRNPGG
QMEGPWCFTQNKNVRMELCDVPSCS
SEQ ID NO: 42; Human ROR2 Ig-like domain
AILHCKVAGNPPPNVRWLKNDAPVVQEPRRIIIRKTEYGSRLRIQDLDTTDTGYYQCVAT
NGMKTITAT

TABLE 10
Exemplary sequences for heavy and light chain
variable domain CDRs for mouse 6E6 antibody.

| SEQ ID | | |
|---|---|---|
| NO: | Name of sequence | Sequence |
| 43. | HCDR1 (IMGT) | GFTFSNYG |
| 44. | HCDR1 (extended) | GFTFSNYGMS |
| 45. | HCDR2 (IMGT) | ISSGGGYT |
| 46. | HCDR2 (extended) | TISSGGGYTHYV |
| 47 | HCDR3 (IMGT) | ARHPRDFSYAMDY |
| 48. | HCDR3 (extended) | ARHPRDFSYAMDY |
| 49. | LCDR1 (IMGT) | QDVGHY |
| 50. | LCDR1 (extended) | KASQDVGHYVA |
| 51. | LCDR2 (IMGT) | WAS |
| 52. | LCDR2 (extended) | WASTRHT |
| 53. | LCDR3 (IMGT) | QQYNIYPWT |
| 54. | LCDR3 (extended) | QQYNIYPWT |

TABLE 11
Exemplary humanized heavy and light chain
variable domain sequences

| SEQ | | |
|---|---|---|
| ID | | |
| NO: | Name of sequence | Sequence |
| 55. | VH3-21*01 | EVQLVESGGGLVKPGGSLRLSCAASGFTFSNYG |
| | (extended) | MSWVRQAPGKGLEWVSTISSGGGYTHYVDSVK |
| | | GRFTISRDNAKNSLYLQMNSLRAEDTAVYYCA |
| | | RHPRDFSYAMDYWGQGTTVTVSS |
| 56. | VH3-11*03 | QVQLLESGGGLVKPGGSLRLSCAASGFTFSNYG |
| | (extended) | MSWIRQAPGKGLEWVSTISSGGGYTHYVDSVK |
| | | GRFTISRDNAKNSLYLQMNSLRAEDTAVYYCA |
| | | RHPRDFSYAMDYWGQGTTVTVSS |
| 57. | VH3-21*01 (IMGT) | EVQLVESGGGLVKPGGSLRLSCAASGFTFSNYG |
| | | MNWVRQAPGKGLEWVSSISSGGGYTYYADSVK |
| | | GRFTISRDNAKNSLYLQMNSLRAEDTAVYYCA |
| | | RHPRDFSYAMDYWGQGTTVTVSS |
| 58. | VH3-11*03 (IMGT) | QVQLLESGGGLVKPGGSLRLSCAASGFTFSNYG |
| | | MSWIRQAPGKGLEWVSYISSGGGYTNYADSVK |
| | | GRFTISRDNAKNSLYLQMNSLRAEDTAVYYCA |
| | | RHPRDFSYAMDYWGQGTTVTVSS |
| 59. | VK1-8*01 (extended) | EVQLVESGGGLVKPGGSLRLSCAASGFTFSNYG |
| | | MSWVRQAPGKGLEWVSTISSGGGYTHYVDSVK |
| | | GRFTISRDNAKNSLYLQMNSLRAEDTAVYYCA |
| | | RHPRDFSYAMDYWGQGTTVTVSS |
| 60. | VK1-8*01 (IMGT) | AIRMTQSPSSFSASTGDRVTITCRASQDVGHYLA |
| | | WYQQKPGKAPKLLIYWASTLQSGVPSRFSGSGS |
| | | GTDFTLTISCLOSEDFATYYCQQYNIYPWTFGQ |
| | | GTKVEIK |
| 61. | VK3-15*01 | EVQLVESGGGLVKPGGSLRLSCAASGFTFSNYG |
| | (extended) | MSWVRQAPGKGLEWVSTISSGGGYTHYVDSVK |
| | | GRFTISRDNAKNSLYLQMNSLRAEDTAVYYCA |
| | | RHPRDFSYAMDYWGQGTTVTVSS |
| 62. | VK3-15*01 (IMGT) | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFGQ |
| | | GTKVEIK |
| 63. | VK1-12*01 | DIQMTQSPSSVSASVGDRVTITCKASQDVGHYV |
| | (extended) | AWYQQKPGKAPKLLIYWASTRHTGVPSRFSGS |
| | | GSGTDFTLTISSLQPEDFATYYCQQYNIYPWTFG |
| | | QGTKVEIK |
| 64 | VK1-12*01 (IMGT) | DIQMTQSPSSVSASVGDRVTITCRASQDVGHYL |
| | | AWYQQKPGKAPKLLIYWASSLQSGVPSRFSGSG |
| | | SGTDFTLTISSLQPEDFATYYCQQYNIYPWTFGQ |
| | | GTKVEIK |

TABLE 12
Exemplary heavy and light chain variable domain sequences for
humanized 6E6 antibodies (Extended CDR sequences in bold)

| SEQ | | |
|---|---|---|
| ID | | |
| NO: | Name of sequence | Sequence |
| 65. | h6E6-001 VH SEQ | EVQLVESGGGLVKPGGSLRLSCAASGFTFSKYG |
| | | ISWVRQAPGKGLEWVSTISSGGGYTHYADSVK |
| | | GRFTISRDNAKNSLYLQMNSLRAEDTAVYYCA |
| | | RHPRDFSYALDYWGQGTTVTVSS |
| 66. | h6E6-001 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 67. | h6E6-002 VH SEQ | EVQLVESGGGLVKPGGSLRLSCAASGFTFSSYG |
| | | ISWVRQAPGKGLEWVSTISSGGGYTHYAESVK |
| | | GRFTISRDNAKNSLYLQMNSLRAEDTAVYYCA |
| | | RHPRDFSYAMDYWGQGTTVTVSS |
| 68. | h6E6-002 VK SEQ | EIVMTQSPATLSVSPGERATLSCRASQDVGHYL |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 69. | h6E6-003 VH SEQ | EVQLVESGGGLVKPGGSLRLSCAASGFTFSQYG |
| | | TSWVRQAPGKGLEWVSTISSGGGYTHYAESVK |
| | | GRFTISRDNAKNSLYLQMNSLRAEDTAVYYCA |
| | | RHPRDFSYAHDYWGQGTTVTVSS |
| 70. | h6E6-003 VK SEQ | EIVMTQSPATLSVSPGERATLSCRASQDVGHYL |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 71. | h6E6-004 VH SEQ | EVQLVESGGGLVKPGGSLRLSCAASGFTFSGYG |
| | | MSWVRQAPGKGLEWVSTISSGGGYTHYAASV |
| | | KGRFTISRDNAKNSLYLQMNSLRAEDTAVYYC |
| | | ARHPRDFSYAFDYWGQGTTVTVSS |
| 72. | h6E6-004 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 73. | h6E6-005 VH SEQ | EVQLVESGGGLVKPGGSLRLSCAASGFTFSGYG |
| | | LSWVRQAPGKGLEWVSTISSGGGYTHYAASV |
| | | KGRFTISRDNAKNSLYLQMNSLRAEDTAVYYC |
| | | ARHPRDFSYAMDYWGQGTTVTVSS |
| 74. | h6E6-005 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 75. | h6E6-006 VH SEQ | EVQLVESGGGLVKPGGSLRLSCAASGFTFSTYG |
| | | MSWVRQAPGKGLEWVSTISSGGGYTHYATSV |
| | | KGRFTISRDNAKNSLYLQMNSLRAEDTAVYYC |
| | | ARHPRDFSYAHDYWGQGTTVTVSS |
| 76. | h6E6-006 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 77. | h6E6-007 VH SEQ | EVQLVESGGGLVKPGGSLRLSCAASGFTFSGYG |
| | | LSWVRQAPGKGLEWVSTISSGGGYTHYAPSVK |
| | | GRFTISRDNAKNSLYLQMNSLRAEDTAVYYCA |
| | | RHPRDFSYAQDYWGQGTTVTVSS |
| 78. | h6E6-007 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 79. | h6E6-008 VH SEQ | EVQLVESGGGLVKPGGSLRLSCAASGFTFSQYG |
| | | TSWVRQAPGKGLEWVSSISSGGGYTHYAESVK |
| | | GRFTISRDNAKNSLYLQMNSLRAEDTAVYYCA |
| | | RHPRDFSYAFDYWGQGTTVTVSS |
| 80. | h6E6-008 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 81. | h6E6-009 VH SEQ | EVQLVESGGGLVKPGGSLRLSCAASGFTFSGYG |
| | | MNWVRQAPGKGLEWVSTISSGGGYTHYAESV |
| | | KGRFTISRDNAKNSLYLQMNSLRAEDTAVYYC |
| | | ARHPRDFSYAADYWGQGTTVTVSS |
| 82. | h6E6-009 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 83. | h6E6-010 VH SEQ | EVQLVESGGGLVKPGGSLRLSCAASGFTFSNYG |
| | | MSWVRQAPGKGLEWVSTISSGGGYTHYVASV |
| | | KGRFTISRDNAKNSLYLQMNSLRAEDTAVYYC |
| | | ARHPRDFSYAMDYWGQGTTVTVSS |
| 84. | h6E6-010 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 85. | h6E6-011 VH SEQ | EVQLVESGGGLVKPGGSLRLSCAASGFTFSGYG |
| | | MSWVRQAPGKGLEWVSTISSGGGYTHYATSV |
| | | KGRFTISRDNAKNSLYLQMNSLRAEDTAVYYC |
| | | ARHPRDFSYAMDYWGQGTTVTVSS |
| 86. | h6E6-011 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 87. | h6E6-012 VH SEQ | EVQLVESGGGLVKPGGSLRLSCAASGFTFSAYG |
| | | ASWVRQAPGKGLEWVSSISSGGGYTHYVESVK |
| | | GRFTISRDNAKNSLYLQMNSLRAEDTAVYYCA |
| | | RHPRDFSYAQDYWGQGTTVTVSS |
| 88. | h6E6-012 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 89. | h6E6-013 VH SEQ | EVQLVESGGGLVKPGGSLRLSCAASGFTFSKYG |
| | | ISWVRQAPGKGLEWVSTISSGGGYTHYAASVK |
| | | GRFTISRDNAKNSLYLQMNSLRAEDTAVYYCA |
| | | RHPRDFSYAQDYWGQGTTVTVSS |
| 90. | h6E6-013 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 91. | h6E6-014 VH SEQ | EVQLVESGGGLVKPGGSLRLSCAASGFTFSSYG |
| | | MNWVRQAPGKGLEWVSTISSGGGYTHYVDSV |
| | | KGRFTISRDNAKNSLYLQMNSLRAEDTAVYYC |
| | | ARHPRDFSYATDYWGQGTTVTVSS |
| 92. | h6E6-014 VK SEQ | EIVMTQSPATLSVSPGERATLSCRASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 93. | h6E6-015 VH SEQ | EVQLVESGGGLVKPGGSLRLSCAASGFTFSNYG |
| | | LSWVRQAPGKGLEWVSTISSGGGYTHYVTSVK |
| | | GRFTISRDNAKNSLYLQMNSLRAEDTAVYYCA |
| | | RHPRDFSYALDYWGQGTTVTVSS |
| 94. | h6E6-015 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 95. | h6E6-016 VH SEQ | EVQLVESGGGLVKPGGSLRLSCAASGFTFSAYG |
| | | MSWVRQAPGKGLEWVSTISSGGGYTHYATSV |
| | | KGRFTISRDNAKNSLYLQMNSLRAEDTAVYYC |
| | | ARHPRDFSYAQDYWGQGTTVTVSS |
| 96. | h6E6-016 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 97. | h6E6-017 VH SEQ | EVQLVESGGGLVKPGGSLRLSCAASGFTFSRYG |
| | | MSWVRQAPGKGLEWVSSISSGGGYTHYAESV |
| | | KGRFTISRDNAKNSLYLQMNSLRAEDTAVYYC |
| | | ARHPRDFSYAMDYWGQGTTVTVSS |
| 98. | h6E6-017 VK SEQ | EIVMTQSPATLSVSPGERATLSCKASQDVGHYL |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 99. | h6E6-018 VH SEQ | EVQLVESGGGLVKPGGSLRLSCAASGFTFSGYG |
| | | VSWVRQAPGKGLEWVSTISSGGGYTHYADSV |
| | | KGRFTISRDNAKNSLYLQMNSLRAEDTAVYYC |
| | | ARHPRDFSYALDYWGQGTTVTVSS |
| 100. | h6E6-018 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 101. | h6E6-019 VH SEQ | EVQLVESGGGLVKPGGSLRLSCAASGFTFSRYG |
| | | VSWVRQAPGKGLEWVSTISSGGGYTHYAASV |
| | | KGRFTISRDNAKNSLYLQMNSLRAEDTAVYYC |
| | | ARHPRDFSYAQDYWGQGTTVTVSS |
| 102. | h6E6-019 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 103. | h6E6-020 VH SEQ | EVQLVESGGGLVKPGGSLRLSCAASGFTFSKYG |
| | | VSWVRQAPGKGLEWVSTISSGGGYTHYAPSVK |
| | | GRFTISRDNAKNSLYLQMNSLRAEDTAVYYCA |
| | | RHPRDFSYAMDYWGQGTTVTVSS |
| 104. | h6E6-020 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 105. | h6E6-021 VH SEQ | EVQLVESGGGLVKPGGSLRLSCAASGFTFSSYG |
| | | MSWVRQAPGKGLEWVSTISSGGGYTHYVASV |
| | | KGRFTISRDNAKNSLYLQMNSLRAEDTAVYYC |
| | | ARHPRDFSYALDYWGQGTTVTVSS |
| 106. | h6E6-021 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 107. | h6E6-022 VH SEQ | EVQLVESGGGLVKPGGSLRLSCAASGFTFSKYG |
| | | TSWVRQAPGKGLEWVSTISSGGGYTHYAPSVK |
| | | GRFTISRDNAKNSLYLQMNSLRAEDTAVYYCA |
| | | RHPRDFSYAMDYWGQGTTVTVSS |
| 108. | h6E6-022 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 109. | h6E6-023 VH SEQ | EVQLVESGGGLVKPGGSLRLSCAASGFTFSSYG |
| | | MSWVRQAPGKGLEWVSTISSGGGYTHYAPSV |
| | | KGRFTISRDNAKNSLYLQMNSLRAEDTAVYYC |
| | | ARHPRDFSYALDYWGQGTTVTVSS |
| 110. | h6E6-023 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 111. | h6E6-024 VH SEQ | EVQLVESGGGLVKPGGSLRLSCAASGFTFSEYG |
| | | MSWVRQAPGKGLEWVSTISSGGGYTHYVESV |
| | | KGRFTISRDNAKNSLYLQMNSLRAEDTAVYYC |
| | | ARHPRDFSYAMDYWGQGTTVTVSS |
| 112. | h6E6-024 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 113. | h6E6-025 VH SEQ | EVQLVESGGGLVKPGGSLRLSCAASGFTFSHYG |
| | | HSWVRQAPGKGLEWVSTISSGGGYTHYVESV |
| | | KGRFTISRDNAKNSLYLQMNSLRAEDTAVYYC |
| | | ARHPRDFSYAMDYWGQGTTVTVSS |
| 114. | h6E6-025 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 115. | h6E6-026 VH SEQ | EVQLVESGGGLVKPGGSLRLSCAASGFTFSRYG |
| | | MSWVRQAPGKGLEWVSTISSGGGYTHYAASV |
| | | KGRFTISRDNAKNSLYLQMNSLRAEDTAVYYC |
| | | ARHPRDFSYAMDYWGQGTTVTVSS |
| 116. | h6E6-026 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 117. | h6E6-027 VH SEQ | EVQLVESGGGLVKPGGSLRLSCAASGFTFSTYG |
| | | LSWVRQAPGKGLEWVSTISSGGGYTHYVDSV |
| | | KGRFTISRDNAKNSLYLQMNSLRAEDTAVYYC |
| | | ARHPRDFSYAMDYWGQGTTVTVSS |
| 118. | h6E6-027 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 119. | h6E6-028 VH SEQ | EVQLVESGGGLVKPGGSLRLSCAASGFTFSPYG |
| | | MSWVRQAPGKGLEWVSTISSGGGYTHYVQSV |
| | | KGRFTISRDNAKNSLYLQMNSLRAEDTAVYYC |
| | | ARHPRDFSYALDYWGQGTTVTVSS |
| 120. | h6E6-028 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 121. | h6E6-029 VH SEQ | EVQLVESGGGLVKPGGSLRLSCAASGFTFSHYG |
| | | VSWVRQAPGKGLEWVSTISSGGGYTHYAQSV |
| | | KGRFTISRDNAKNSLYLQMNSLRAEDTAVYYC |
| | | ARHPRDFSYAQDYWGQGTTVTVSS |
| 122. | h6E6-029 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 123. | h6E6-030 VH SEQ | EVQLVESGGGLVKPGGSLRLSCAASGFTFSSYG |
| | | MSWVRQAPGKGLEWVSTISSGGGYTHYAASV |
| | | KGRFTISRDNAKNSLYLQMNSLRAEDTAVYYC |
| | | ARHPRDFSYAMDYWGQGTTVTVSS |
| 124. | h6E6-030 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 125. | h6E6-031 VH SEQ | EVQLVESGGGLVKPGGSLRLSCAASGFTFSSYG |
| | | MSWVRQAPGKGLEWVSTISSGGGYTHYAESV |
| | | KGRFTISRDNDKNSLYLQMNSLRAEDTAVYYC |
| | | ARHPRDFSYAIDYWGQGTTVTVSS |
| 126. | h6E6-031 VK SEQ | EIVMTQSPATLSVSPGERATLSCRASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 127. | h6E6-032 VH SEQ | EVQLVESGGGLVKPGGSLRLSCAASGFTFSNYG |
| | | TSWVRQAPGKGLEWVSTISSGGGYTHYAESVK |
| | | GRFTISRDNAKNSLYLQMNSLRAEDTAVYYCA |
| | | RHPRDFSYAMDYWGQGTTVTVSS |
| 128. | h6E6-032 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 129. | h6E6-033 VH SEQ | EVQLVESGGGLVKPGGSLRLSCAASGFTFSTYG |
| | | MSWVRQAPGKGLEWVSTISSGGGYTHYVTSV |
| | | KGRFTISRDNAKNSLYLQMNSLRAEDTAVYYC |
| | | ARHPRDFSYAMDYWGQGTTVTVSS |
| 130. | h6E6-033 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 131. | h6E6-034 VH SEQ | EVQLVESGGGLVKPGGSLRLSCAASGFTFSSYG |
| | | MSWVRQAPGKGLEWVSTISSGGGYTHYVHSV |
| | | KGRFTISRDNAKNSLYLQMNSLRAEDTAVYYC |
| | | ARHPRDFSYAMDYWGQGTTVTVSS |
| 132. | h6E6-034 VK SEQ | EIVMTPSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 133. | h6E6-035 VH SEQ | EVQLVESGGGLVKPGGSLRLSCAASGFTFSSYG |
| | | ISWVRQAPGKGLEWVSTISSGGGYTHYADSVK |
| | | GRFTISRDNAKNSLYLQMNSLRAEDTAVYYCA |
| | | RHPRDFSYAHDYWGQGTTVTVSS |
| 134. | h6E6-035 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 135. | h6E6-036 VH SEQ | EVQLVESGGGLVKPGGSLRLSCAASGFTFSAYG |
| | | HSWVRQAPGKGLEWVSTISSGGGYTHYADSV |
| | | KGRFTISRDNAKNSLYLQMNSLRAEDTAVYYC |
| | | ARHPRDFSYAQDYWGQGTTVTVSS |
| 136 | h6E6-036 VK SEQ | EIVMTQSPATLSVSPGERATLSCKASQDVGHYL |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 137. | h6E6-037 VH SEQ | EVQLVESGGGLVKPGGSLRLSCAASGFTFSAYG |
| | | MSWVRQAPGKGLEWVSTISSGGGYTHYATSV |
| | | KGRFTISRDNAKNSLYLQMNSLRAEDTAVYYC |
| | | ARHPRDFSYAQDYWGQGTTVTVSS |
| 138. | h6E6-037 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 139. | h6E6-038 VH SEQ | EVQLVESGGGLVKPGGSLRLSCAASGFTFSRYG |
| | | MSWVRQAPGKGLEWVSTISSGGGYTHYAPSV |
| | | KGRFTISRDNAKNSLYLQMNSPRAEDTAVYYC |
| | | ARHPRDFSYAQDYWGQGTTVTVSS |
| 140. | h6E6-038 VK SEQ | EIVMTQSPATLSVSPGERATLSCKASQDVGHYL |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 141. | h6E6-039 VH SEQ | EVQLVESGGGLVKPGGSLRLSCAASGFTFSNYG |
| | | QSWVRQAPGKGLEWVSTISSGGGYTHYADSV |
| | | KGRFTISRDNAKNSLYLQMNSLRAEDTAVYYC |
| | | ARHPRDFSYAVDYWGQGTTVTVSS |
| 142. | h6E6-039 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 143. | h6E6-040 VH SEQ | EVQLVESGGGLVKPGGSLRLSCAASGFTFSSYG |
| | | HSWVRQAPGKGLEWVSTISSGGGYTHYAPSVK |
| | | GRFTISRDNAKNSLYLQMNSLRAEDTAVYYCA |
| | | RHPRDFSYAHDYWGQGTTVTVSS |
| 144. | h6E6-040 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 145. | h6E6-041 VH SEQ | EVQLVESGGGLVKPGGSLRLSCAASGFTFSGYG |
| | | VSWVRQAPGKGLEWVSTISSGGGYTHYAPSVK |
| | | GRFTISRDNAKNSLYLQMNSLRAEDTAVYYCA |
| | | RHPRDFSYAQDYWGQGTTVTVSS |
| 146. | h6E6-041 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 147. | h6E6-042 VH SEQ | EVQLVESGGGLVKPGGSLRLSCAASGFTFSGYG |
| | | LSWVRQAPGKGLEWVSTISSGGGYTHYVESVK |
| | | GRFTISRDNAKNSLYLQMNSLRAEDTAVYYCA |
| | | RHPRDFSYASDYWGQGTTVTVSS |
| 148. | h6E6-042 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 149. | h6E6-043 VH SEQ | EVQLVESGGGLVKPGGSLRLSCAASGFTFSSYG |
| | | TSWVRQAPGKGLEWVSTISSGGGYTHYATSVK |
| | | GRFTISRDNAKNSLYLQMNSLRAEDTAVYYCA |
| | | RHPRDFSYAMDYWGQGTTVTVSS |
| 150. | h6E6-043 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 151. | h6E6-044 VH SEQ | EVQLVESGGGLVKPGGSLRLSCAASGFTFSTYG |
| | | SSWVRQAPGKGLEWVSTISSGGGYTHYAQSVK |
| | | GRFTISRDNAKNSLYLQMNSLRAEDTAVYYCA |
| | | RHPRDFSYALDYWGQGTTVTVSS |
| 152. | h6E6-044 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 153. | h6E6-045 VH SEQ | EVQLVESGGGLVKPGGSLRLSCAASGFTFSGYG |
| | | MSWVRQAPGKGLEWVSTISSGGGYTHYVQSV |
| | | KGRFTISRDNAKNSLYLQMNSLRAEDTAVYYC |
| | | ARHPRDFSYAMDYWGQGTTVTVSS |
| 154. | h6E6-045 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 155. | h6E6-046 VH SEQ | EVQLVESGGGLVKPGGSLRLSCAASGFTFSRYG |
| | | ISWVRQAPGKGLEWVSTISSGGGYTHYATSVK |
| | | GRFTISRDNAKNSLYLQMNSLRAEDTAVYYCA |
| | | RHPRDFSYAMDYWGQGTTVTVSS |
| 156. | h6E6-046 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 157. | h6E6-047 VH SEQ | EVQLVESGGGLVKPGGSLRLSCAASGFTFSKYG |
| | | MSWVRQAPGKGPEWVSTISSGGGYTHYATSV |
| | | KGRFTISRDNAKNSLYLQMNSLRAEDTAVYYC |
| | | ARHPRDFSYALDYWGQGTTVTVSS |
| 158. | h6E6-047 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 159. | h6E6-048 VH SEQ | EVQLVESGGGLVKPGGSLRLSCAASGFTFSNYG |
| | | VSWVRQAPGKGLEWVSTISSGGGYTHYVQSV |
| | | KGRFTISRDNAKNSLYLQMNSLRAEDTAVYYC |
| | | ARHPRDFSYALDYWGQGTTVTVSS |
| 160. | h6E6-048 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 161. | h6E6-049 VH SEQ | EVQLVESGGGLVKPGGSLRLSCAASGFTFSNYG |
| | | MSWVRQAPGKGLEWVSSISSGGGYTHYAESV |
| | | KGRFTISRDNAKNSLYLQMNSLRAEDTAVYYC |
| | | ARHPRDFSYAMDYWGQGTTVTVSS |
| 162. | h6E6-049 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 163. | h6E6-050 VH SEQ | EVQLVESGGGLVKPGGSLRLSCAASGFTFSRYG |
| | | MSWVRQAPGKGLEWVSTISSGGGYTHYVDSV |
| | | KGRFTISRDNAKNSLYLQMNSLRAEDTAVYYC |
| | | ARHPRDFSYAHDYWGQGTTVTVSS |
| 164. | h6E6-050 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 165. | h6E6-051 VH SEQ | EVQLVESGGGLVKPGGSLRLSCAASGFTFSNYG |
| | | MSWVRQAPGKGLEWVSTISSGGGYTHYVDSV |
| | | KGRFTISRDNAKNSLYLQMNSLRAEDTAVYYC |
| | | ARHPRDFSYALDYWGQGTTVTVSS |
| 166. | h6E6-051 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 167. | h6E6-052 VH SEQ | EVQLVESGGGLVKPGGSLRLSCAASGFTFSHYG |
| | | TSWVRQAPGKGLEWVSTISSGGGYTHYAESVK |
| | | GRFTISRDNAKNSLYLQMNSLRAEDTAVYYCA |
| | | RHPRDFSYAIDYWGQGTTVTVSS |
| 168. | h6E6-052 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 169. | h6E6-053 VH SEQ | EVQLVESGGGLVKPGGSLRLSCAASGFTFSKYG |
| | | VSWVRQAPGKGLEWVSTISSGGGYTHYVDSV |
| | | KGRFTISRDNAKNSLYLQMNSLRAEDTAVYYC |
| | | ARHPRDFSYAHDYWGQGTTVTVSS |
| 170. | h6E6-053 VK SEQ | EIVMTQSPATLSVSPGERATLSCKASQDVGHYV |
| | | AWYQQKPGQAPRLLIYWASTRHTGIPARFSGS |
| | | GSGTEFTLTISSLKSEDFAVYYCQQYNIYPWTF |
| | | GQGTKVEIK |
| 171. | h6E6-054 VH SEQ | EVQLVESGGGLVKPGGSLRLSCAASGFTFSNYG |
| | | VSWVRQAPGKGLEWVSTISSGGGYTHYVTSVK |
| | | GRFTISRDNAKNSLYLQMNSLRAEDTAVYYCA |
| | | RHPRDFSYALDYWGQGTTVTVSS |
| 172. | h6E6-054 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 173. | h6E6-055 VH SEQ | EVQLVESGGGLVKPGGSLRLSCAASGFTFSQYG |
| | | HSWVRQAPGKGLEWVSTISSGGGYTHYAESV |
| | | KGRFTISRDNAKNSLYLQMNSLRAEDTAVYYC |
| | | ARHPRDFSYAQDYWGQGTTVTVSS |
| 174. | h6E6-055 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 175. | h6E6-056 VH SEQ | EVQLVESGGGLVKPGGSLRLSCAASGFTFSKYG |
| | | TSWVRQAPGKGLEWVSTISSGGGYTHYAPSVK |
| | | GRFTISRDNAKNSLYLQMNSLRAEDTAVYYCA |
| | | RHPRDFSYAQDYWGQGTTVTVSS |
| 176. | h6E6-056 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 177. | h6E6-057 VH SEQ | EVQLVESGGGLVKPGGSLRLSCAASGFTFSHYG |
| | | VSWVRQAPGKGLEWVSTISSGGGYTHYVASV |
| | | KGRFTISRDNAKNSLYLQMNSLRAEDTAVYYC |
| | | ARHPRDFSYALDYWGQGTTVTVSS |
| 178. | h6E6-057 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWAFG |
| | | QGTKVEIK |
| 179. | h6E6-058 VH SEQ | EVQLVESGGGLVKPGGSLRLSCAASGFTFSVYG |
| | | LSWVRQAPGKGLEWVSTISSGGGYTQYAPSVK |
| | | GRFTISRDNAKNSLYLQMNSLRAEDTAVYYCA |
| | | RHPRDFSYAFDYWGQGTTVTVSS |
| 180. | h6E6-058 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 181. | h6E6-059 VH SEQ | EVQLVESGGGLVKPGGSLRLSCAASGFTFSGYG |
| | | LSWVRQAPGKGLEWVSTISSGGGYTHYAPSVK |
| | | GRFTISRDNAKNSLYLQMNSLRAEDTAVYYCA |
| | | RHPRDFSYAFDYWGQGTTVTVSS |
| 182. | h6E6-059 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 183. | h6E6-060 VH SEQ | EVQLVESGGGLVKPGGSLRLSCAASGFTFSRYG |
| | | LSWVRQAPGKGLEWVSTISSGGGYTHYAASV |
| | | KGRFTISRDNAKNSLYLQMNSLRAEDTAVYYC |
| | | ARHPRDFSYAMDYWGQGTTVTVSS |
| 184. | h6E6-060 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 185. | h6E6-061 VH SEQ | EVQLVESGGGLVKPGGSLRLSCAASGFTFSNYG |
| | | QSWVRQAPGKGLEWVSTISSGGGYTHYAPSVK |
| | | GRFTISRDNAKNSLYLQMNSLRAEDTAVYYCA |
| | | RHPRDFSYAFDYWGQGTTVTVSS |
| 186. | h6E6-061 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 187. | h6E6-062 VH SEQ | EVQLVESGGGLVKPGGSLRLSCAASGFTFSSYG |
| | | HSWVRQAPGKGLEWVSTISSGGGYTYYAESVK |
| | | GRFTISRDNAKNSLYLQMNSLRAEDTAVYYCA |
| | | RHPRDFSYAHDYWGQGTTVTVSS |
| 188. | h6E6-062 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 189. | h6E6-063 VH SEQ | EVQLVESGGGLVKPGGSLRLSCAASGFTFSAYG |
| | | TSWVRQAPGKGLEWVSTISSGGGYTYYAASVK |
| | | GRFTISRDNAKNSLYLQMNSLRAEDTAVYYCA |
| | | RHPRDFSYAIDYWGQGTTVTVSS |
| 190. | h6E6-063 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 191. | h6E6-064 VH SEQ | EVQLVESGGGLVKPGGSLRLSCAASGFTFSHYG |
| | | ISWVRQAPGKGLEWVSSISSGGGYTHYAESVK |
| | | GRFTISRDNAKNSLYLQMNSLRAEDTAVYYCA |
| | | RHPRDFSYAMDYWGQGTTVTVSS |
| 192. | h6E6-064 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 193. | h6E6-065 VH SEQ | EVQLVESGGGLVKPGGSLRLSCAASGFTFSGYG |
| | | MSWVRQAPGKGLEWVSTISSGGGYTHYVDSV |
| | | KGRFTISRDNAKNSLYLQMNSLRAEDTAVYYC |
| | | ARHPRDFSYALDYWGQGTTVTVSS |
| 194. | h6E6-065 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 195. | h6E6-066 VH SEQ | EVQLVESGGGLVKPGGSLRLSCAASGFTFSAYG |
| | | SSWVRQAPGKGLEWVSTISSGGGYTHYAPSVK |
| | | GRFTISRDNAKNSLYLQMNSLRAEDTAVYYCA |
| | | RHPRDFSYAHDYWGQGTTVTVSS |
| 196. | h6E6-066 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 197. | h6E6-067 VH SEQ | EVQLVESGGGLVKPGGSLRLSCAASGFTFSHYG |
| | | ASWVRQAPGKGLEWVSTISSGGGYTHYAGSV |
| | | KGRFTISRDNAKNSLYLQMNSLRAEDTAVYYC |
| | | ARHPRDFSYAFDYWGQGTTVTVSS |
| 198. | h6E6-067 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 199. | h6E6-068 VH SEQ | EVQLVESGGGLVKPGGSLRLSCAASGFTFSSYG |
| | | ISWVRQAPGKGLEWVSTISSGGGYTHYAHSVK |
| | | GRFTISRDNAKNSLYLQMNSLRAEDTAVYYCA |
| | | RHPRDFSYALDYWGQGTTVTVSS |
| 200. | h6E6-068 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 201. | h6E6-069 VH SEQ | EVQLVESGGGLVKPGGSLRLSCAASGFTFSKYG |
| | | VSWVRQAPGKGLEWVSTISSGGGYTHYVESVK |
| | | GRFTISRDNAKNSLYLQMNSLRAEDTAVYYCA |
| | | RHPRDFSYAMDYWGQGTTVTVSS |
| 202. | h6E6-069 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 203. | h6E6-070 VH SEQ | EVQLVESGGGLVKPGGSLRLSCAASGFTFSQYG |
| | | HSWVRQAPGKGLEWVSTISSGGGYTHYAHSV |
| | | KGRFTISRDNAKNSLYLQMNSLRAEDTAVYYC |
| | | ARHPRDFSYANDYWGQGTTVTVSS |
| 204. | h6E6-070 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 205. | h6E6-071 VH SEQ | EVQLVESGGGLVKPGGSLRLSCAASGFTFSHYG |
| | | MSWVRQAPGKGLEWVSTISSGGGYTHYAESV |
| | | KGRFTISRDNAKNSLYLQMNSLRAEDTAVYYC |
| | | ARHPRDFSYAFDYWGQGTTVTVSS |
| 206. | h6E6-071 VK SEQ | EIVMTQSPATLSVSPGERATLSCKASQDVGHYL |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 207. | h6E6-072 VH SEQ | EVQLVESGGGLVKPGGSLRLSCAASGFTFSQYG |
| | | HSWVRQAPGKGLEWVSTISSGGGYTHYADSV |
| | | KGRFTISRDNAKNSLYLQMNSLRAEDTAVYYC |
| | | ARHPRDFSYAQDYWGQGTTVTVSS |
| 208. | h6E6-072 VK SEQ | EIVMTQSPATLSVSPGERATLSCKASQDVGHYL |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 209. | h6E6-073 VH SEQ | EVQLVESGGGLVKPGGSLRLSCAASGFTFSPYG |
| | | MSWVRQAPGKGLEWVSTISSGGGYTYYAASV |
| | | KGRFTISRDNAKNSLYLQMNSLRAEDTAVYYC |
| | | ARHPRDFSYAMDYWGQGTTVTVSS |
| 210. | h6E6-073 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 211. | h6E6-074 VH SEQ | EVQLVESGGGLVKPGGSLRLSCAASGFTFSKYG |
| | | TSWVRQAPGKGLEWVSTISSGGGYTHYAPSVK |
| | | GRFTISRDNAKNSLYLQMNSLRAEDTAVYYCA |
| | | RHPRDFSYALDYWGQGTTVTVSS |
| 212. | h6E6-074 VK SEQ | EIVMTQSPATLSVSPGERATLSCKASQDVGHYL |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 213. | h6E6-075 VH SEQ | EVQLVESGGGLVKPGGSLRLSCAASGFTFSAYG |
| | | ASWVRQAPGKGLEWVSTISSGGGYTHYAGSV |
| | | KGRFTISRDNAKNSLYLQMNSLRAEDTAVYYC |
| | | ARHPRDFSYAQDYWGQGTTVTVSS |
| 214. | h6E6-075 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 215. | h6E6-076 VH SEQ | EVQLVESGGGLVKPGGSLRLSCAASGFTFSGYG |
| | | MSWVRQAPGKGLEWVSTISSGGGYTHYVESV |
| | | KGRFTISRDNAKNSLYLQMNSLRAEDTAVYYC |
| | | ARHPRDFSYALDYWGQGTTVTVSS |
| 216. | h6E6-076 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 217. | h6E6-077 VH SEQ | EVQLVESGGGLVKPGGSLRLSCAASGFTFSHYG |
| | | TSWVRQAPGKGLEWVSTISSGGGYTHYAPSVK |
| | | GRFTISRDNAKNSLYLQMNSLRAEDTAVYYCA |
| | | RHPRDFSYAIDYWGQGTTVTVSS |
| 218. | h6E6-077 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 219. | h6E6-078 VH SEQ | EVQLVESGGGLVKPGGSLRLSCAASGFTFSAYG |
| | | SSWVRQAPGKGLEWVSTISSGGGYTHYAPSVK |
| | | GRFTISRDNAKNSLYLQMNSLRAEDTAVYYCA |
| | | RHPRDFSYAMDYWGQGTTVTVSS |
| 220. | h6E6-078 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 221. | h6E6-079 VH SEQ | EVQLVESGGGLVKPGGSLRLSCAASGFTFSSYG |
| | | QSWVRQAPGKGLEWVSTISSGGGYTHYAPSVK |
| | | GRFTISRDNAKNSLYLQMNSLRAEDTAVYYCA |
| | | RHPRDFSYAMDYWGQGTTVTVSS |
| 222. | h6E6-079 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 223. | h6E6-080 VH SEQ | EVQLVESGGGLVKPGGSLRLSCAASGFTFSHYG |
| | | LNWVRQAPGKGLEWVSTISSGGGYTHYAPSV |
| | | KGRFTISRDNAKNSLYLQMNSLRAEDTAVYYC |
| | | ARHPRDFSYANDYWGQGTTVTVSS |
| 224. | h6E6-080 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 225. | h6E6-081 VH SEQ | EVQLVESGGGLVKPGGSLRLSCAASGFTFSSYG |
| | | MNWVRQAPGKGLEWVSTISSGGGYTHYADSV |
| | | KGRFTISRDNAKNSLYLQMNSLRAEDTAVYYC |
| | | ARHPRDFSYAQDYWGQGTTVTVSS |
| 226. | h6E6-081 VK SEQ | EIVMTQSPATLSVSPGERATLSCRASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 227. | h6E6-082 VH SEQ | EVQLVESGGGLVKPGGSLRLSCAASGFTFSKYG |
| | | MSWVRQAPGKGLEWVSTISSGGGYTHYADSV |
| | | KGRFTISRDNAKNSLYLQMNSLRAEDTAVYYC |
| | | ARHPRDFSYAMDYWGQGTTVTVSS |
| 228. | h6E6-082 VK SEQ | EIVMTQSPATLSVSPGERATLSCKASQDVGHYL |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 229. | h6E6-083 VH SEQ | EVQLVESGGGLVKPGGSLRLSCAASGFTFSVYG |
| | | MSWVRQAPGKGLEWVSTISSGGGYTHYAPSV |
| | | KGRFTISRDNAKNSLYLQMNSLRAEDTAVYYC |
| | | ARHPRDFSYAMDYWGQGTTVTVSS |
| 230. | h6E6-083 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 231. | h6E6-084 VH SEQ | EVQLVESGGGLVKPGGSLRLSCAASGFTFSQYG |
| | | MSWVRQAPGKGLEWVSTISSGGGYTHYVQSV |
| | | KGRFTISRDNAKNSLYLQMNSLRAEDTAVYYC |
| | | ARHPRDFSYAQDYWGQGTTVTVSS |
| 232. | h6E6-084 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 233. | h6E6-085 VH SEQ | EVQLVESGGGLVKPGGSLRLSCAASGFTFSHYG |
| | | ANWVRQAPGKGLEWVSTISSGGGYTHYATSV |
| | | KGRFTISRDNAKNSLYLQMNSLRAEDTAVYYC |
| | | ARHPRDFSYAVDYWGQGTTVTVSS |
| 234. | h6E6-085 VK SEQ | EIVMTQSPATLSVSPGERATLSCRASQDVGHYV |
| | | AWYQQKPGQAPRLLIYWASTSATGIPARFSGSG |
| | | SGTEFTLTISSLQSEDFAVYYCQQYNIYPWTFG |
| | | QGTKVEIK |
| 235. | h6E6-086 VH SEQ | EVQLVESGGGLVKPGGSLRLSCAASGFTFSQYG |
| | | MSWVRQAPGKGLEWVSTISSGGGYTHYVTSV |
| | | KGRFTISRDNAKNSLYLQMNSLRAEDTAVYYC |
| | | ARHPRDFSYAKDYWGQGTTVTVSS |
| 236. | h6E6-086 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 237. | h6E6-087 VH SEQ | EVQLVESGGGLVKPGGSLRLSCAASGFTFSKYG |
| | | MSWVRQAPGKGLEWVSTISSGGGYTHYVHSV |
| | | KGRFTISRDNAKNSLYLQMNSLRAEDTAVYYC |
| | | ARHPRDFSYANDYWGQGTTVTVSS |
| 238. | h6E6-087 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 239. | h6E6-088 VH SEQ | EVQLVESGGGLVKPGGSLRLSCAASGFTFSGYG |
| | | ASWVRQAPGKGLEWVSTISSGGGYTHYAPSVK |
| | | GRFTISRDNAKNSLYLQMNSLRAEDTAVYYCA |
| | | RHPRDFSYAQDYWGQGTTVTVSS |
| 240. | h6E6-088 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 241. | h6E6-089 VH SEQ | EVQLVESGGGLVKPGGSLRLSCAASGFTFSDYG |
| | | ISWVRQAPGKGLEWVSTISSGGGYTHYVQSVK |
| | | GRFTISRDNAKNSLYLQMNSLRAEDTAVYYCA |
| | | RHPRDFSYAMDYWGQGTTVTVSS |
| 242. | h6E6-089 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 243. | h6E6-090 VH SEQ | EVQLVESGGGLVKPGGSLRLSCAASGFTFSTYG |
| | | TSWVRQAPGKGLEWVSTISSGGGYTHYAPSVK |
| | | GRFTISRDNAKNSLYLQMNSLRAEDTAVYYCA |
| | | RHPRDFSYALDYWGQGTTVTVSS |
| 244. | h6E6-090 VK SEQ | EIVMTQSPATLSVSPGERATLSCKASQDVGHYL |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 245. | h6E6-091 VH SEQ | EVQLVESGGGLVKPGGSLRLSCAASGFTFSAYG |
| | | LSWVRQAPGKGLEWVSTISSGGGYTHYAQSV |
| | | KGRFTISRDNAKNSLYLQMNSLRAEDTAVYYC |
| | | ARHPRDFSYAFDYWGQGTTVTVSS |
| 246. | h6E6-091 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 247. | h6E6-092 VH SEQ | EVQLVESGGGLVKPGGSLRLSCAASGFTFSSYG |
| | | ISWVRQAPGKGLEWVSTISSGGGYTHYAANVK |
| | | GRFTISRDNAKNSLYLQMNSLRAEDTAVYYCA |
| | | RHPRDFSYAHDYWGQGTTVTVSS |
| 248. | h6E6-092 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 249. | h6E6-093 VH SEQ | EVQLVESGGGLVKPGGSLRLSCAASGFTFSGYG |
| | | MNWVRQAPGKGLEWVSTISSGGGYTHYVESV |
| | | KGRFTISRDNAKNSLYLQMNSLRAEDTAVYYC |
| | | ARHPRDFSYAMDYWGQGTTVTVSS |
| 250. | h6E6-093 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 251. | h6E6-094 VH SEQ | EVQLVESGGGLVKPGGSLRLSCAASGFTFSRYG |
| | | QSWVRQAPGKGLEWVSTISSGGGYTHYAPSVK |
| | | GRFTISRDNAKNSLYLQMNSLRAEDTAVYYCA |
| | | RHPRDFSYALDYWGQGTTVTVSS |
| 252. | h6E6-094 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 253. | h6E6-095 VH SEQ | EVQLVESGGGLVKPGGSLRLSCAASGFTFSGYG |
| | | VSWVRQAPGKGLEWVSTISSGGGYTHYVDSV |
| | | KGRFTISRDNAKNSLYLQMNSLRAEDTAVYYC |
| | | ARHPRDFSYALDYWGQGTTVTVSS |
| 254. | h6E6-095 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 255. | h6E6-096 VH SEQ | EVQLVESGGGLVKPGGSLRLSCAASGFTFSNYG |
| | | VSWVRQAPGKGLEWVSTISSGGGYTHYAPSVK |
| | | GRFTISRDNAKNSLYLQMNSLRAEDTAVYYCA |
| | | RHPRDFSYAQDYWGQGTTVTVSS |
| 256. | h6E6-096 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 257. | h6E6-097 VH SEQ | EVQLVESGGGLVKPGGSLRLSCAASGFTFSAYG |
| | | MSWVRQAPGKGLEWVSTISSGGGYTHYVESV |
| | | KGRFTISRDNAKNSLYLQMNSLRAEDTAVYYC |
| | | ARHPRDFSYAMDYWGQGTTVTVSS |
| 258. | h6E6-097 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 259. | h6E6-098 VH SEQ | EVQLVESGGGLVKPGGSLRLSCAASGFTFSAYG |
| | | VSWVRQAPGKGLEWVSTISSGGGYTYYVESVK |
| | | GRFTISRDNAKNSLYLQMNSLRAEDTAVYYCA |
| | | RHPRDFSYAMDYWGQGTTVTVSS |
| 260. | h6E6-098 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 261. | h6E6-099 VH SEQ | EVQLVESGGGLVKPGGSLRLSCAASGFTFSNYG |
| | | MNWVRQAPGKGLEWVSTISSGGGYTHYAPSV |
| | | KGRFTISRDNAKNSLYLQMNSLRAEDTAVYYC |
| | | ARHPRDFSYAMDYWGQGTTVTVSS |
| 262. | h6E6-099 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 263. | h6E6-100 VH SEQ | EVQLVESGGGLVKPGGSLRLSCAASGFTFSHYG |
| | | LSWVRQAPGKGLEWVSTISSGGGYTHYVDSV |
| | | KGRFTISRDNAKNSLYLQMNSLRAEDTAVYYC |
| | | ARHPRDFSYANDYWGQGTTVTVSS |
| 264. | h6E6-100 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 265. | h6E6-101 VH SEQ | EVQLVESGGGLVKPGGSZRLSCAASGFTFSFYG |
| | | MSWVRQAPGKGLEWVSTISSGGGYTYYAESV |
| | | KGRFTISRDNAKNSLYLQMNSLRAEDTAVYYC |
| | | ARHPRDFSYAIDYWGQGTTVTVSS |
| 266. | h6E6-101 VK SEQ | EIVMTQSPATLSVSPGERATLSCRASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 267. | h6E6-102 VH SEQ | EVQLVESGGGLVKPGGSLRLSCAASGFTFSKYG |
| | | LSWVRQAPGKGLEWVSTISSGGGYTHYVESVK |
| | | GRFTISRDNAKNSLYLQMNSLRAEDTAVYYCA |
| | | RHPRDFSYASDYWGQGTTVTVSS |
| 268. | h6E6-102 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 269. | h6E6-103 VH SEQ | EVQLVESGGGLVKPGGSLRLSCAASGFTFSNYG |
| | | MSWVRQAPGKGLEWVSTISSGGGYTHYVDSV |
| | | KGRFTISRDNAKNSLYLQMNSLRAEDTAVYYC |
| | | ARHPRDFSYAMDYWGQGTTVTVSS |
| 270. | h6E6-103 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 271. | h6E6-104 VH SEQ | EVQLVESGGGLVKPGGSLRLSCAASGFTFSSYG |
| | | VSWVRQAPGKGLEWVSTISSGGGYTHYVESVK |
| | | GRFTISRDNAKNSLYLQMNSLRAEDTAVYYCA |
| | | RHPRDFSYAQDYWGQGTTVTVSS |
| 272. | h6E6-104 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 273. | h6E6-105 VH SEQ | EVQLVESGGGLVKPGGSLRLSCAASGFTFSTYG |
| | | HSWVRQAPGKGLEWVSTISSGGGYTHYVASV |
| | | KGRFTISRDNAKNSLYLQMNSLRAEDTAVYYC |
| | | ARHPRDFSYAMDYWGQGTTVTVSS |
| 274. | h6E6-105 VK SEQ | EIVMTQSPATLSVSPGERATLSCRASQDVGHYV |
| | | AWYQQKPGQAPRLLIYWASTRHTGIPARFSGS |
| | | GSGTEFTLTISSLQSEDFAVYYCQQYNIYPWTF |
| | | GQGIKVEIK |
| 275. | h6E6-106 VH SEQ | EVQLVESGGGLVKPGGSLRLSCAASGFTFSSYG |
| | | ISWVRQAPGKGLEWVSTISSGGGYTYYVDSVK |
| | | GRFTISRDNAKNSLYLQMNSLRAEDTAVYYCA |
| | | RHPRDFSYALDYWGQGTTVTVSS |
| 276. | h6E6-106 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 277. | h6E6-107 VH SEQ | EVQLVESGGGLVKPGGSLRLSCAASGFTFSTYG |
| | | ASWVRQAPGKGLEWVSTISSGGGYTHYAPSVK |
| | | GRFTISRDNAKNSLYLQMNSLRAEDTAVYYCA |
| | | RHPRDFSYALDYWGQGTTVTVSS |
| 278. | h6E6-107 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 279. | h6E6-108 VH SEQ | EVQLVESGGGLVKPGGSLRLSCAASGFTFSRYG |
| | | VSWVRQAPGKGLEWVSTISSGGGYTHYAESVK |
| | | GRFTISRDNAKNSLYLQMNSLRAEDTAVYYCA |
| | | RHPRDFSYAMDYWGQGTTVTVSS |
| 280. | h6E6-108 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 281. | h6E6-109 VH SEQ | EVQLVESGGGLVKPGGSLRLSCAASGFTFSNYG |
| | | TSWVRQAPGKGLEWVSTISSGGGYTHYVTSVK |
| | | GRFTISRDNAKNSLYLQMNSLRAEDTAVYYCA |
| | | RHPRDFSYAMDYWGQGTTVTVSS |
| 282. | h6E6-109 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 283. | h6E6-110 VH SEQ | EVQLVESGGGLVKPGGSLRLSCAASGFTFSHYG |
| | | VSWVRQAPGKGLEWVSTISSGGGYTHYAESVK |
| | | GRFTISRDNAKNSLYLQMNSLRAEDTAVYYCA |
| | | RHPRDFSYAQDYWGQGTTVTVSS |
| 284. | h6E6-110 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 285. | h6E6-111 VH SEQ | EVQLVESGGGLVKPGGSLRLSCAASGFTFSTYG |
| | | MNWVRQAPGKGLEWVSTISSGGGYTHYAESV |
| | | KGRFTISRDNAKNSLYLQMNSLRAEDTAVYYC |
| | | ARHPRDFSYAVDYWGQGTTVTVSS |
| 286. | h6E6-111 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 287. | h6E6-112 VH SEQ | EVQLVESGGGLVKPGGSLRLSCAASGFTFSSYG |
| | | ISWVRQAPGKGLEWVSTISSGGGYTHYAASVK |
| | | GRFTISRDNAKNSLYLQMNSLRAEDTAVYYCA |
| | | RHPRDFSYALDYWGQGTTVTVSS |
| 288. | h6E6-112 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 289. | h6E6-113 VH SEQ | EVQLVESGGGLVKPGGSLRLSCAASGFTFSNYG |
| | | MSWVRQAPGKGLEWVSTISSGGGYTHYAPSV |
| | | KGRFTISRDNAKNSLYLQMNSLRAEDTAVYYC |
| | | ARHPRDFSYAMDYWGQGTTVTVSS |
| 290. | h6E6-113 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 291. | h6E6-114 VH SEQ | EVQLVESGGGLVKPGGSLRLSCAASGFTFSKYG |
| | | MSWVRQAPGKGLEWVSTISSGGGYTHYVTSV |
| | | KGRFTISRDNAKNSLYLQMNSLRAEDTAVYYC |
| | | ARHPRDFSYAMDYWGQGTTVTVSS |
| 292. | h6E6-114 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 293. | h6E6-115 VH SEQ | EVQLVESGGGLVKPGGSLRLSCAASGFTFSGYG |
| | | MSWVRQAPGKGLEWVSTISSGGGYTHYVTSV |
| | | KGRFTISRDNAKNSLYLQMNSLRAEDTAVYYC |
| | | ARHPRDFSYALDYWGQGTTVTVSS |
| 294. | h6E6-115 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 295. | h6E6-116 VH SEQ | EVQLVESGGGLVKPGGSLRLSCAASGFTFSTYG |
| | | VSWVRQAPGKGLEWVSTISSGGGYTHYAGSV |
| | | KGRFTISRDNAKNSLYLQMNSLRAEDTAVYYC |
| | | ARHPRDFSYALDYWGQGTTVTVSS |
| 296. | h6E6-116 VK SEQ | EIVMTQSPATLSVSPGERATLSCRASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |
| 297. | h6E6-117 VH SEQ | EVQLVESGGGLVKPGGSLRLSCAASGFTFSVYG |
| | | ISWVRQAPGKGLEWVSTISSGGGYTHYAPSVK |
| | | GRFTISRDNAKNSLYLQMNSLRAEDTAVYYCA |
| | | RHPRDFSYAMDYWGQGTTVTVSS |
| 298. | h6E6-117 VK SEQ | EIVMTQSPATLSVSPGERATLSCKASQDVGHYV |
| | | AWYQQKPGQAPRLLIYWASTRATGIPARFSGSG |
| | | SGTEFTLTISSLQSEDFAVYYCQQYNIYPWTFG |
| | | QGTKVEIK |
| 299. | h6E6-118 VH SEQ | EVQLVESGGGLVKPGGSLRLSCAASGFTFSRYG |
| | | HSWVRQAPGKGLEWVSTISSGGGYTYYAESVK |
| | | GRFTISRDNAKNSLYLQMNSLRAEDTAVYYCA |
| | | RHPRDFSYAHDYWGQGTTVTVSS |
| 300. | h6E6-118 VK SEQ | EIVMTQSPATLSVSPGERATLSCRASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 301. | h6E6-119 VH SEQ | EVQLVESGGGLVKPGGSLRLSCAASGFTFSAYG |
| | | TSWVRQAPGKGLEWVSTISSGGGYTHYVHSV |
| | | KGRFTISRDNAKNSLYLQMNSLRAEDTAVYYC |
| | | ARHPRDFSYAMDYWGQGTTVTVSS |
| 302. | h6E6-119 VK SEQ | EIVMTQSPATLSVSPGERATLSCKASQDVGHYV |
| | | AWYQQKPGQAPRLLIYWASTRHTGIPARFSGS |
| | | GSGTEFTLTISSLQSEDFAVYYCQQYNIYPWTF |
| | | GQGTKVEIK |
| 303. | h6E6-120 VH SEQ | EVQLVESGGGLVKPGGSLRLSCAASGFTFSSYG |
| | | MSWVRQAPGKGLEWVSTISSGGGYTHYVQSV |
| | | KGRFTISRDNAKNSLYLQMNSLRAEDTAVYYC |
| | | ARHPRDFSYAMDYWGQGTTVTVSS |
| 304. | h6E6-120 VK SEQ | EIVMTQSPATLSVSPGERATLSCKASQDVGHYL |
| | | AWYQQKPGQAPRLLIYWASTRATGIPARFSGSG |
| | | SGTEFTLTISSLQSEDFAVYYCQQYNIYPWTFG |
| | | QGTKVEIK |

TABLE 13
Exemplary sequences for heavy and light chain variable
domain extended CDRs for humanized 6E6 antibodies.

| SEQ ID | | |
|---|---|---|
| NO: | Name of sequence | Sequence |
| 305. | h6E6-070 VH CDR H1 | GFTFSQYGHS |
| 306. | h6E6-070 VH CDR H2 | TISSGGGYTHYA |
| 307. | h6E6-070 VH CDR H3 | ARHPRDFSYANDY |
| 308. | h6E6-070 VL CDR L1 | RASQDVGHYLA |
| 309. | h6E6-070 VL CDR L2 | WASTRAT |
| 310. | h6E6-070 VL CDR L3 | QQYNIYPWT |
| 311. | h6E6-116 VH CDR H1 | GFTFSTYGVS |
| 312. | h6E6-116 VH CDR H2 | TISSGGGYTHYA |
| 313. | h6E6-116 VH CDR H3 | ARHPRDFSYALDY |
| 314. | h6E6-116 VL CDR L1 | RASQDVGHYLA |
| 315. | h6E6-116 VL CDR L2 | WASTRAT |
| 316. | h6E6-116 VL CDR L3 | QQYNIYPWT |
| 317. | h6E6-007 VH CDR H1 | GFTFSGYGLS |
| 318. | h6E6-007 VH CDR H2 | TISSGGGYTHYA |
| 319. | h6E6-007 VH CDR H3 | ARHPRDFSYAQDY |
| 320. | h6E6-007 VL CDR L1 | KASQDVGHYVA |
| 321. | h6E6-007 VL CDR L2 | WASTRAT |
| 322. | h6E6-007 VL CDR L3 | QQYNIYPWT |
| 323 | h6E6-029 VH CDR H1 | GFTFSHYGVS |
| 324. | h6E6-029 VH CDR H2 | TISSGGGYTHYA |
| 325. | h6E6-029 VH CDR H3 | ARHPRDFSYAQDY |
| 326. | h6E6-029 VL CDR L1 | KASQDVGHYVA |
| 327. | h6E6-029 VL CDR L2 | WASTRHT |
| 328 | h6E6-029 VL CDR L3 | QQYNIYPWT |
| 329. | h6E6-040 VH CDR H1 | GFTFSSYGHS |
| 330. | h6E6-040 VH CDR H2 | TISSGGGYTHYA |
| 331. | h6E6-040 VH CDR H3 | ARHPRDFSYAHDY |
| 332. | h6E6-040 VL CDR L1 | RASQDVGHYVA |
| 333. | h6E6-040 VL CDR L2 | WASTRAT |
| 334. | h6E6-040 VL CDR L3 | QQYNIYPWT |
| 335. | h6E6-055 VH CDR H1 | GFTFSQYGHS |
| 336 | h6E6-055 VH CDR H2 | TISSGGGYTHYA |
| 337. | h6E6-055 VH CDR H3 | ARHPRDFSYAQDY |
| 338. | h6E6-055 VL CDR L1 | KASQDVGHYLA |
| 339. | h6E6-055 VL CDR L2 | WASTRAT |
| 340. | h6E6-055 VL CDR L3 | QQYNIYPWT |
| 341. | h6E6-056 VH CDR H1 | GFTFSKYGTS |
| 342. | h6E6-056 VH CDR H2 | TISSGGGYTHYA |
| 343. | h6E6-056 VH CDR H3 | ARHPRDFSYAQDY |
| 344 | h6E6-056 VL CDR L1 | RASQDVGHYVA |
| 345. | h6E6-056 VL CDR L2 | WASTRAT |
| 346. | h6E6-056 VL CDR L3 | QQYNIYPWT |
| 347. | h6E6-057 VH CDR H1 | GFTFSHYGVS |
| 348. | h6E6-057 VH CDR H2 | TISSGGGYTHYV |
| 349. | h6E6-057 VH CDR H3 | ARHPRDFSYALDY |
| 350. | h6E6-057 VL CDR L1 | KASQDVGHYVA |
| 351. | h6E6-057 VL CDR L2 | WASTRAT |
| 352. | h6E6-057 VL CDR L3 | QQYNIYPWA |
| 353. | h6E6-058 VH CDR H1 | GFTFSVYGLS |
| 354 | h6E6-058 VH CDR H2 | TISSGGGYTQYA |
| 355. | h6E6-058 VH CDR H3 | ARHPRDFSYAFDY |
| 356. | h6E6-058 VL CDR L1 | KASQDVGHYLA |
| 357. | h6E6-058 VL CDR L2 | WASTRAT |
| 358. | h6E6-058 VL CDR L3 | QQYNIYPWT |
| 359. | h6E6-063 VH CDR H1 | GFTFSAYGTS |
| 360. | h6E6-063 VH CDR H2 | TISSGGGYTYYA |
| 361. | h6E6-063 VH CDR H3 | ARHPRDFSYAIDY |
| 362 | h6E6-063 VL CDR L1 | RASQDVGHYLA |
| 363. | h6E6-063 VL CDR L2 | WASTRAT |
| 364. | h6E6-063 VL CDR L3 | QQYNIYPWT |
| 365. | h6E6-068 VH CDR H1 | GFTFSSYGIS |
| 366. | h6E6-068 VH CDR H2 | TISSGGGYTHYA |
| 367. | h6E6-068 VH CDR H3 | ARHPRDFSYALDY |
| 368. | h6E6-068 VL CDR L1 | KASQDVGHYLA |
| 369. | h6E6-068 VL CDR L2 | WASTRAT |
| 370. | h6E6-068 VL CDR L3 | QQYNIYPWT |
| 371. | h6E6-072 VH CDR H1 | GFTFSQYGHS |
| 372. | h6E6-072 VH CDR H2 | TISSGGGYTHYA |
| 373. | h6E6-072 VH CDR H3 | ARHPRDFSYAQDY |
| 374. | h6E6-072 VL CDR L1 | KASQDVGHYLA |
| 375. | h6E6-072 VL CDR L2 | WASTRHT |
| 376. | h6E6-072 VL CDR L3 | QQYNIYPWT |
| 377. | h6E6-077 VH CDR H1 | GFTFSHYGTS |
| 378. | h6E6-077 VH CDR H2 | TISSGGGYTHYA |
| 379. | h6E6-077 VH CDR H3 | ARHPRDFSYAIDY |
| 380. | h6E6-077 VL CDR L1 | KASQDVGHYVA |
| 381. | h6E6-077 VL CDR L2 | WASTRAT |
| 382. | h6E6-077 VL CDR L3 | QQYNIYPWT |
| 383. | h6E6-102 VH CDR H1 | GFTFSKYGLS |
| 384. | h6E6-102 VH CDR H2 | TISSGGGYTHYV |
| 385. | h6E6-102 VH CDR H3 | ARHPRDFSYASDY |
| 386. | h6E6-102 VL CDR LI | KASQDVGHYVA |
| 387. | h6E6-102 VL CDR L2 | WASTRAT |
| 388. | h6E6-102 VL CDR L3 | QQYNIYPWT |
| 389. | h6E6-107 VH CDR H1 | GFTFSTYGAS |
| 390. | h6E6-107 VH CDR H2 | TISSGGGYTHYA |
| 391. | h6E6-107 VH CDR H3 | ARHPRDFSYALDY |
| 392. | h6E6-107 VL CDR L1 | KASQDVGHYVA |
| 393. | h6E6-107 VL CDR L2 | WASTRAT |
| 394. | h6E6-107 VL CDR L3 | QQYNIYPWT |
| 395. | h6E6-066 VH CDR H1 | GFTFSAYGSS |
| 396. | h6E6-066 VH CDR H2 | TISSGGGYTHYA |
| 397 | h6E6-066 VH CDR H3 | ARHPRDFSYAHDY |
| 398. | h6E6-066 VL CDR L1 | KASQDVGHYVA |
| 399. | h6E6-066 VL CDR L2 | WASTRHT |
| 400. | h6E6-066 VL CDR L3 | QQYNIYPWT |

### P EMBODIMENTS

P Embodiment 1. A method of identifying an anti-ROR2 antibody, the method comprising: (i) contacting an antibody with a first ROR2 polypeptide comprising a histidine at a position corresponding to position 349 of SEQ ID NO:34; (ii) detecting said antibody binding to said first ROR2 polypeptide; (iii) contacting said antibody with a second ROR2 polypeptide not comprising a histidine at a position corresponding to position 349 of SEQ ID NO: 34; and (iv) detecting said antibody not binding to said second ROR2 polypeptide, thereby identifying an anti-ROR2 antibody.

P Embodiment 2. A method of identifying an anti-ROR2 antibody, the method comprising: (i) contacting an antibody with a first ROR2 polypeptide comprising an aspartic acid at a position corresponding to position 354 of SEQ ID NO:34; (ii) detecting said antibody binding to said first ROR2 polypeptide; (iii) contacting said antibody with a second ROR2 polypeptide not comprising an aspartic acid at a position corresponding to position 354 of SEQ ID NO:34; and (iv) detecting said antibody not binding to said second ROR2 polypeptide, thereby identifying an anti-ROR2 antibody.

P Embodiment 3. A method of identifying an anti-ROR2 antibody, the method comprising: (i) contacting an antibody with a first ROR2 polypeptide comprising a methionine at a position corresponding to position 386 of SEQ ID NO:34; (ii) detecting said antibody binding to said first ROR2 polypeptide; (iii) contacting said antibody with a second ROR2 polypeptide not comprising a methionine at a position corresponding to position 386 of SEQ ID NO:34; and (iv) detecting said antibody not binding to said second ROR2 polypeptide, thereby identifying an anti-ROR2 antibody.

P Embodiment 4. An anti-tyrosine kinase-like orphan receptor 2 (ROR2) antibody comprising a heavy chain variable domain and a light chain variable domain, wherein said heavy chain variable domain comprises the sequence of SEQ ID NO: 1 and wherein said light chain variable domain comprises the sequence of SEQ ID NO: 2; wherein said heavy chain variable domain comprises the sequence of SEQ ID NO: 3 and wherein said light chain variable domain comprises the sequence of SEQ ID NO: 4: wherein said heavy chain variable domain comprises the sequence of SEQ ID NO: 5 and wherein said light chain variable domain comprises the sequence of SEQ ID NO: 6; wherein said heavy chain variable domain comprises the sequence of SEQ ID NO: 7 and wherein said light chain variable domain comprises the sequence of SEQ ID NO: 8; wherein said heavy chain variable domain comprises the sequence of SEQ ID NO: 9 and wherein said light chain variable domain comprises the sequence of SEQ ID NO: 10, wherein said heavy chain variable domain comprises the sequence of SEQ ID NO: 11 and wherein said light chain variable domain comprises the sequence of SEQ ID NO: 12; wherein said heavy chain variable domain comprises the sequence of SEQ ID NO: 13 and wherein said light chain variable domain comprises the sequence of SEQ ID NO: 14: wherein said heavy chain variable domain comprises the sequence of SEQ ID NO: 15 and wherein said light chain variable domain comprises the sequence of SEQ ID NO: 16; wherein said heavy chain variable domain comprises the sequence of SEQ ID NO: 17 and wherein said light chain variable domain comprises the sequence of SEQ ID NO: 18; wherein said heavy chain variable domain comprises the sequence of SEQ ID NO: 19 and wherein said light chain variable domain comprises the sequence of SEQ ID NO: 20: wherein said heavy chain variable domain comprises the sequence of SEQ ID NO: 21 and wherein said light chain variable domain comprises the sequence of SEQ ID NO: 22: wherein said heavy chain variable domain comprises the sequence of SEQ ID NO: 23 and wherein said light chain variable domain comprises the sequence of SEQ ID NO: 24; wherein said heavy chain variable domain comprises the sequence of SEQ ID NO: 25 and wherein said light chain variable domain comprises the sequence of SEQ ID NO: 26; wherein said heavy chain variable domain comprises the sequence of SEQ ID NO: 27 and wherein said light chain variable domain comprises the sequence of SEQ ID NO: 28; wherein said heavy chain variable domain comprises the sequence of SEQ ID NO: 29 and wherein said light chain variable domain comprises the sequence of SEQ ID NO: 30; or wherein said heavy chain variable domain comprises the sequence of SEQ ID NO: 31 and wherein said light chain variable domain comprises the sequence of SEQ ID NO: 32.

P Embodiment 5. The antibody of P embodiment 4, wherein said antibody binds the Kringle domain of human ROR2.

P Embodiment 6. The antibody of P embodiment 4, wherein said antibody binds a cancer cell.

P Embodiment 7. The antibody of P embodiment 6, wherein said cancer cell is a breast cancer cell, ovarian cancer cell, pancreatic cancer cell, cervical cancer cell, gastric cancer cell, renal cancer cell, head and neck cancer cell, bone cancer cell, skin cancer cell or prostate cancer cell.

P Embodiment 8. The antibody of P embodiment 4, wherein said antibody is attached to a therapeutic agent.

P Embodiment 9. The antibody of P embodiment 4, wherein said antibody is attached to a diagnostic agent.

P Embodiment 10. A method of treating cancer in a subject in need thereof, said method comprising administering to a subject a therapeutically effective amount of an antibody of 4.

P Embodiment 11. A method of inhibiting metastasis of a ROR2-expressing cancer in a subject in need thereof, said method comprising administering to a subject a therapeutically effective amount of an antibody of P embodiment 4.

P Embodiment 12. A chimeric antigen receptor comprising: (i) an antibody region comprising any one of the light chain variable domain and heavy chain variable domain pairings set forth by P embodiment 4; and (ii) a transmembrane domain.

P Embodiment 13. An anti-ROR2 antibody capable of binding the same epitope as the antibody set forth by P embodiment 4.

### Embodiments

Embodiment 1. An anti-tyrosine kinase-like orphan receptor 2 (ROR2) antibody comprising: (i) a CDR H1 comprising an amino acid sequence of formula: G-F-T-F-S-X1-Y-G-X2-X3 (I), wherein X1 is A, D, E, F, G, H, I, K, L, N, P, Q, R, S, T, V, W, or Y, X2 is A, E, F, G, H, I, K, L, M, P, Q, R, S, T, V, W, or Y, X3 is S or N. and wherein if X2 is M and X3 is S X1 is not N; (ii) a CDR H2 comprising an amino acid sequence of formula: X4-I-S-S-G-G-G-Y-T-X5-Y-X6 (II), wherein X4 is T or S, X5 is H or Y, X6 is V or A, and wherein if X5 is H and X6 is V X4 is not T; (iii) a CDR H3 comprising an amino acid sequence of formula: D-F-S-Y-A-X7-D-Y-W-G (1II); wherein X7 is A, D, E, F, G, H, I, K, L, M, P, Q, R, S, T, V, W, or Y; (iv) a CDR L1 comprising an amino acid sequence of formula: X8-A-S-Q-D-V-G-H-Y-X9-A (IV) wherein X8 is K or R, X9 is V or L, and wherein if X8 is K X9 is not V; (v) a CDR L2 comprising an amino acid sequence of formula: W-A-S-T-R-X10-T (V) wherein X10 is H or A, and wherein if X8 is K and X9 is V X10 is not H; and (vi) a CDR L3 comprising the sequence of SEQ ID NO:53.

Embodiment 2. The antibody of embodiment 1, further comprising a FR H3 comprising an amino acid sequence of formula: Y-T-H-Y-V-X11-S-V-K-G (VI), wherein X11 is A, D, E, G, H, P, Q, T, or V.

Embodiment 3. The antibody of embodiment 1 or embodiment 2, wherein X1 is S, G, N, K, A, or H.

Embodiment 4. The antibody of any one of embodiments 1 to 3, wherein X1 is S.

Embodiment 5. The antibody of any one of embodiments 1 to 4, wherein X2 is M, V, T, I or L.

Embodiment 6. The antibody of any one of embodiments 1 to 5, wherein X2 is V.

Embodiment 7. The antibody of any one of embodiments 1 to 6, wherein X7 is M, L or Q.

Embodiment 8. The antibody of any one of embodiments 1 to 7, wherein X7 is L.

Embodiment 9. The antibody of any one of embodiments 2 to 8, wherein X11 is E, P, D, A, or T.

Embodiment 10. The antibody of any one of embodiments 2 to 9, wherein X11 is E.

Embodiment 11. The antibody of any one of embodiments 2 to 10, wherein X1 is not N, X2 is not M. X7 is not M or X11 is not D.

Embodiment 12. The antibody of any one of embodiments 2 to 11, wherein X1 is not N, X2 is not M, X7 is not M and X11 is not D.

Embodiment 13. The antibody of embodiment 1, wherein said antibody comprises: a CDR H1 as set forth in SEQ ID NO:305, a CDR H2 as set forth in SEQ ID NO:306 and a CDR H3 as set forth in SEQ ID NO:307; and a CDR L1 as set forth in SEQ ID NO:308, a CDR L2 as set forth in SEQ ID NO:309, and a CDR L3 as set forth in SEQ ID NO:310.

Embodiment 14. The antibody of embodiment 1, wherein said antibody comprises: a CDR H1 as set forth in SEQ ID NO:311, a CDR H2 as set forth in SEQ ID NO:312 and a CDR H3 as set forth in SEQ ID NO:313; and a CDR L1 as set forth in SEQ ID NO:314, a CDR L2 as set forth in SEQ ID NO:315, and a CDR L3 as set forth in SEQ ID NO:316.

Embodiment 15. The antibody of embodiment 1, wherein said antibody comprises: a CDR H1 as set forth in SEQ ID NO:317, a CDR H2 as set forth in SEQ ID NO:318 and a CDR H3 as set forth in SEQ ID NO:319; and a CDR L1 as set forth in SEQ ID NO:320, a CDR L2 as set forth in SEQ ID NO:321, and a CDR L3 as set forth in SEQ ID NO:322.

Embodiment 16. The antibody of embodiment 1, wherein said antibody comprises: a CDR H1 as set forth in SEQ ID NO:323, a CDR H2 as set forth in SEQ ID NO:324 and a CDR H3 as set forth in SEQ ID NO:325; and a CDR L1 as set forth in SEQ ID NO:326, a CDR L2 as set forth in SEQ ID NO:327, and a CDR L3 as set forth in SEQ ID NO:328.

Embodiment 17. The antibody of embodiment 1, wherein said antibody comprises: a CDR H1 as set forth in SEQ ID NO:329, a CDR H2 as set forth in SEQ ID NO:330 and a CDR H3 as set forth in SEQ ID NO:31; and a CDR L1 as set forth in SEQ ID NO:332, a CDR L2 as set forth in SEQ ID NO:333, and a CDR L3 as set forth in SEQ ID NO:334.

Embodiment 18. The antibody of embodiment 1, wherein said antibody comprises: a CDR H1 as set forth in SEQ ID NO:335, a CDR H2 as set forth in SEQ ID NO:336 and a CDR H3 as set forth in SEQ ID NO:337; and a CDR L1 as set forth in SEQ ID NO:338, a CDR L2 as set forth in SEQ ID NO:339, and a CDR L3 as set forth in SEQ ID NO:340.

Embodiment 19. The antibody of embodiment 1, wherein said antibody comprises: a CDR H1 as set forth in SEQ ID NO:341, a CDR H2 as set forth in SEQ ID NO:342 and a CDR H3 as set forth in SEQ ID NO:343; and a CDR L1 as set forth in SEQ ID NO:344, a CDR L2 as set forth in SEQ ID NO:345, and a CDR L3 as set forth in SEQ ID NO:346.

Embodiment 20. The antibody of embodiment 1, wherein said antibody comprises: a CDR H1 as set forth in SEQ ID NO:347, a CDR H2 as set forth in SEQ ID NO:348 and a CDR H3 as set forth in SEQ ID NO:349; and a CDR L1 as set forth in SEQ ID NO:350, a CDR L2 as set forth in SEQ ID NO:351, and a CDR L3 as set forth in SEQ ID NO:352.

Embodiment 21. The antibody of embodiment 1, wherein said antibody comprises: a CDR H1 as set forth in SEQ ID NO:353, a CDR H2 as set forth in SEQ ID NO:354 and a CDR H3 as set forth in SEQ ID NO:355; and a CDR L1 as set forth in SEQ ID NO:356, a CDR L2 as set forth in SEQ ID NO:357, and a CDR L3 as set forth in SEQ ID NO:358.

Embodiment 22. The antibody of embodiment 1, wherein said antibody comprises: a CDR H1 as set forth in SEQ ID NO:359, a CDR H2 as set forth in SEQ ID NO:360 and a CDR H3 as set forth in SEQ ID NO:361; and a CDR L1 as set forth in SEQ ID NO:362, a CDR L2 as set forth in SEQ ID NO:363, and a CDR L3 as set forth in SEQ ID NO:364.

Embodiment 23. The antibody of embodiment 1, wherein said antibody comprises: a CDR H1 as set forth in SEQ ID NO:365, a CDR H2 as set forth in SEQ ID NO:366 and a CDR H3 as set forth in SEQ ID NO:367; and a CDR L1 as set forth in SEQ ID NO:368, a CDR L2 as set forth in SEQ ID NO:369, and a CDR L3 as set forth in SEQ ID NO:370.

Embodiment 24. The antibody of embodiment 1, wherein said antibody comprises: a CDR H1 as set forth in SEQ ID NO:371, a CDR H2 as set forth in SEQ ID NO:372 and a CDR H3 as set forth in SEQ ID NO:373; and a CDR L1 as set forth in SEQ ID NO:374, a CDR L2 as set forth in SEQ ID NO:375, and a CDR L3 as set forth in SEQ ID NO:376.

Embodiment 25. The antibody of embodiment 1, wherein said antibody comprises: a CDR H1 as set forth in SEQ ID NO:377, a CDR H2 as set forth in SEQ ID NO:378 and a CDR H3 as set forth in SEQ ID NO:379; and a CDR L1 as set forth in SEQ ID NO:380, a CDR L2 as set forth in SEQ ID NO:381, and a CDR L3 as set forth in SEQ ID NO:382.

Embodiment 26. The antibody of embodiment 1, wherein said antibody comprises: a CDR H1 as set forth in SEQ ID NO:383, a CDR H2 as set forth in SEQ ID NO:384 and a CDR H3 as set forth in SEQ ID NO:385; and a CDR L1 as set forth in SEQ ID NO:386, a CDR L2 as set forth in SEQ ID NO:387, and a CDR L3 as set forth in SEQ ID NO:388.

Embodiment 27. The antibody of embodiment 1, wherein said antibody comprises: a CDR H1 as set forth in SEQ ID NO:389, a CDR H2 as set forth in SEQ ID NO:390 and a CDR H3 as set forth in SEQ ID NO:391; and a CDR L1 as set forth in SEQ ID NO:392, a CDR L2 as set forth in SEQ ID NO:393, and a CDR L3 as set forth in SEQ ID NO:394.

Embodiment 28. The antibody of any one of embodiments 1 to 27, wherein said antibody is a humanized antibody.

Embodiment 29. The antibody of any one of embodiments 1 to 27, wherein said antibody is a Fab′ fragment.

Embodiment 30. The antibody of any one of embodiments 1 to 27, wherein said antibody is a single chain antibody (scFv).

Embodiment 31. The antibody of any one of embodiments 1 to 27, wherein said antibody is a chimeric antibody.

Embodiment 32. The antibody of any one of embodiments 1 to 27, wherein said antibody is an IgG.

Embodiment 33. The antibody of any one of embodiments 1 to 27, wherein said antibody is an IgG1 or an IgG2.

Embodiment 34. The antibody of any one of embodiments 1 to 33, wherein said antibody is capable of binding a ROR2 protein.

Embodiment 35. The antibody of any one of embodiments 1 to 33, wherein said antibody is capable of binding the extracellular domain of said ROR2 protein.

Embodiment 36. The antibody of embodiment 35, wherein said extracellular domain is a Kringle domain.

Embodiment 37. The antibody of any one of embodiments 34 to 36, wherein said ROR2 protein is expressed on a cell.

Embodiment 38. The antibody of embodiment 37, wherein said cell is a cancer cell.

Embodiment 39. The antibody of embodiment 38, wherein said cancer cell is a breast cancer cell, an ovarian cancer cell, a pancreatic cancer cell, a cervical cancer cell, a gastric cancer cell, a renal cancer cell, a head and neck cancer cell, a bone cancer cell, a skin cancer cell or a prostate cancer cell.

Embodiment 40. The antibody of any one of embodiments 34 to 39, wherein said antibody is capable of binding said ROR2 protein with an equilibrium dissociation constant (KD) from about 0.02 nM to about 6 nM.

Embodiment 41. The antibody of any one of embodiments 1 to 40, wherein said antibody is attached to a therapeutic agent.

Embodiment 42. The antibody of any one of embodiments 1 to 40, wherein said antibody is attached to a diagnostic agent.

Embodiment 43. A method of treating cancer in a subject in need thereof, said method comprising administering to a subject a therapeutically effective amount of an antibody of any one of embodiments 1-41.

Embodiment 44. The method of embodiment 43, wherein said cancer is breast cancer, ovarian cancer, pancreatic cancer, cervical cancer, gastric cancer, renal cancer, head and neck cancer, bone cancer, skin cancer or prostate cancer.

Embodiment 45. A method of detecting a ROR2 expressing cell, said method comprising (i) contacting a ROR2-expressing cell with an antibody of any one of embodiments 1-40; and (ii) detecting binding of said antibody to a ROR2 protein expressed by said cell.

Embodiment 46. The method of embodiment 45, wherein said antibody is attached to a detectable moiety.

Embodiment 47. The method of embodiment 45, wherein said ROR2-expressing cell is in a subject.

Embodiment 48. The method of embodiment 47, wherein said subject is a subject having or being at risk of having cancer.

Embodiment 49. The method of embodiment 48, wherein said cancer is breast cancer, ovarian cancer, pancreatic cancer, cervical cancer, gastric cancer, renal cancer, head and neck cancer, bone cancer, skin cancer or prostate cancer.

Embodiment 50. The method of embodiment 45, wherein said contacting occurs in vitro.

Embodiment 51. A method of delivering a therapeutic agent to a ROR2 expressing cell, said method comprising contacting a ROR2 expressing cell with an antibody of any one of embodiments 1-40, wherein said antibody is attached to a therapeutic agent.

Embodiment 52. The method of embodiment 51, wherein said therapeutic agent is an anti-cancer agent.

Embodiment 53. The method of embodiment 51, wherein said ROR2-expressing cell is in a subject.

Embodiment 54. The method of embodiment 51, wherein said subject is a subject having or being at risk of having cancer.

Embodiment 55. The method of embodiment 54, wherein said cancer is breast cancer, ovarian cancer, pancreatic cancer, cervical cancer, gastric cancer, renal cancer, head and neck cancer, bone cancer, skin cancer or prostate cancer.

Embodiment 56. The method of embodiment 51, wherein said contacting occurs in vitro.

Embodiment 57. A method of inhibiting migration of a ROR2-expressing cell, said method comprising contacting a ROR2 expressing cell with an antibody of any one of embodiments 1-40.

Embodiment 58. The method of embodiment 57, wherein said ROR2 expressing cell is in a subject having or being at risk of having cancer.

Embodiment 59. The method of embodiment 58, wherein said cancer is breast cancer, ovarian cancer, pancreatic cancer, cervical cancer, gastric cancer, renal cancer, head and neck cancer, bone cancer, skin cancer or prostate cancer.

Embodiment 60. The method of embodiment 57, wherein said contacting occurs in vitro.

Embodiment 61. The method of embodiment 57, wherein said ROR2 expressing cell is a cancer cell.

Embodiment 62. The method of embodiment 61, wherein said cancer cell is a breast cancer cell, an ovarian cancer cell, a pancreatic cancer cell, a cervical cancer cell, a gastric cancer cell, a renal cancer cell, a head and neck cancer cell, a bone cancer cell, a skin cancer cell or a prostate cancer cell.

Embodiment 63. An anti-ROR2 antibody, wherein said anti-ROR2 antibody binds the same epitope as an anti-ROR2 antibody of any one of embodiments 1-40.

Embodiment 64. A chimeric antigen receptor comprising (i) an anti-ROR2 antibody of any one of embodiments 1-40 and (ii) a transmembrane domain.

Embodiment 65. A method of treating cancer in a subject in need thereof said method comprising, administering a therapeutically effective amount of a chimeric antigen receptor of embodiment 64 to said subject.

Embodiment 66. The method of embodiment 65, wherein said cancer is breast cancer, ovarian cancer, pancreatic cancer, cervical cancer, gastric cancer, renal cancer, head and neck cancer, bone cancer, skin cancer or prostate cancer.

What is claimed is:

1. An anti-tyrosine kinase-like orphan receptor 2 (ROR2) antibody comprising:
(i) a CDR H1 comprising an amino acid sequence of formula: 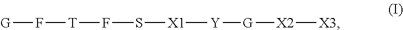
wherein
X1 is A, D, E, F, G, H, I, K, L, N, P, Q, R, S, T, V, W, or Y,
X2 is A, E, F, G, H, I, K, L, M, P, Q, R, S, T, V, W, or Y,
X3 is S or N, and
wherein if X2 is M and X3 is S X1 is not N;
(ii) a CDR H2 comprising an amino acid sequence of formula: 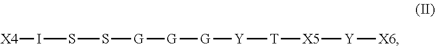
wherein
X4 is T or S,
X5 is Hor Y,
X6 is V or A, and
wherein if X5 is H and X6 is V X4 is not T;
(iii) a CDR H3 comprising an amino acid sequence of formula: 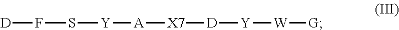
wherein
X7 is A, D, E, F, G, H, I, K, L, M, P, Q, R, S, T, V, W, or Y;
(iv) a CDR L1 comprising an amino acid sequence of formula: 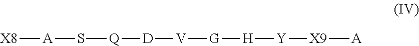
wherein
X8 is K or R,
X9 is V or L, and
wherein if X8 is K X9 is not V;
(v) a CDR L2 comprising an amino acid sequence of formula: 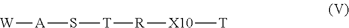
wherein
X10 is H or A, and
wherein if X8 is K and X9 is V X10 is not H; and
(vi) a CDR L3 comprising the sequence of SEQ ID NO:53.

2. The antibody of claim 1, further comprising a FR H3 comprising an amino acid sequence of formula: 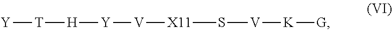
wherein
X11 is A, D, E, G, H, P, Q, T, or V.

3. The antibody of claim 2, wherein X1 is S, G, N, K, A, or H.

4. The antibody of claim 3, wherein X1 is S.

5. The antibody of claim 4, wherein X² is M, V, T, I or L.

6. The antibody of oc claim 5, wherein X² is V.

7. The antibody of claim 6, wherein X7 is M, L or Q.

8. The antibody of claim 7, wherein X7 is L.

9. The antibody of claim 8, wherein X11 is E, P, D, A, or T.

10. The antibody of any one of claims 2 to 9, wherein X11 is E.

11. The antibody of claim 2, wherein X1 is not N, X2 is not M, X7 is not M or X11 is not D.

12. The antibody of claim 2, wherein X1 is not N, X2 is not M, X7 is not M and X11 is not D.

13. The antibody of claim 1, wherein said antibody comprises:
a CDR H1 as set forth in SEQ ID NO:305, a CDR H2 as set forth in SEQ ID NO: 306 and a CDR H3 as set forth in SEQ ID NO:307; and
a CDR L1 as set forth in SEQ ID NO:308, a CDR L2 as set forth in SEQ ID NO: 309, and a CDR L3 as set forth in SEQ ID NO:310.

14. The antibody of claim 1, wherein said antibody comprises:
a CDR H1 as set forth in SEQ ID NO:311, a CDR H2 as set forth in SEQ ID NO: 312 and a CDR H3 as set forth in SEQ ID NO:313; and
a CDR L1 as set forth in SEQ ID NO:314, a CDR L2 as set forth in SEQ ID NO: 315, and a CDR L3 as set forth in SEQ ID NO:316.

15. The antibody of claim 1, wherein said antibody comprises:
a CDR H1 as set forth in SEQ ID NO:317, a CDR H2 as set forth in SEQ ID NO: 318 and a CDR H3 as set forth in SEQ ID NO:319; and
a CDR L1 as set forth in SEQ ID NO:320, a CDR L2 as set forth in SEQ ID NO: 321, and a CDR L3 as set forth in SEQ ID NO:322.

16. The antibody of claim 1, wherein said antibody comprises:
a CDR H1 as set forth in SEQ ID NO:323, a CDR H2 as set forth in SEQ ID NO: 324 and a CDR H3 as set forth in SEQ ID NO:325; and
a CDR L1 as set forth in SEQ ID NO:326, a CDR L2 as set forth in SEQ ID NO: 327, and a CDR L3 as set forth in SEQ ID NO:328.

17. The antibody of claim 1, wherein said antibody comprises:
a CDR H1 as set forth in SEQ ID NO:329, a CDR H2 as set forth in SEQ ID NO: 330 and a CDR H3 as set forth in SEQ ID NO:31; and
a CDR L1 as set forth in SEQ ID NO:332, a CDR L2 as set forth in SEQ ID NO: 333, and a CDR L3 as set forth in SEQ ID NO:334.

18. The antibody of claim 1, wherein said antibody comprises:
a CDR H1 as set forth in SEQ ID NO:335, a CDR H2 as set forth in SEQ ID NO: 336 and a CDR H3 as set forth in SEQ ID NO:337; and
a CDR L1 as set forth in SEQ ID NO:338, a CDR L2 as set forth in SEQ ID NO: 339, and a CDR L3 as set forth in SEQ ID NO:340.

19. The antibody of claim 1, wherein said antibody comprises:
a CDR H1 as set forth in SEQ ID NO:341, a CDR H2 as set forth in SEQ ID NO: 342 and a CDR H3 as set forth in SEQ ID NO:343; and
a CDR L1 as set forth in SEQ ID NO:344, a CDR L2 as set forth in SEQ ID NO: 345, and a CDR L3 as set forth in SEQ ID NO:346.

20. The antibody of claim 1, wherein said antibody comprises:
a CDR H1 as set forth in SEQ ID NO:347, a CDR H2 as set forth in SEQ ID NO: 348 and a CDR H3 as set forth in SEQ ID NO:349; and
a CDR L1 as set forth in SEQ ID NO:350, a CDR L2 as set forth in SEQ ID NO: 351, and a CDR L3 as set forth in SEQ ID NO:352.

21. The antibody of claim 1, wherein said antibody comprises:
a CDR H1 as set forth in SEQ ID NO:353, a CDR H2 as set forth in SEQ ID NO: 354 and a CDR H3 as set forth in SEQ ID NO:355; and
a CDR L1 as set forth in SEQ ID NO:356, a CDR L2 as set forth in SEQ ID NO: 357, and a CDR L3 as set forth in SEQ ID NO:358.

22. The antibody of claim 1, wherein said antibody comprises:
a CDR H1 as set forth in SEQ ID NO:359, a CDR H2 as set forth in SEQ ID NO: 360 and a CDR H3 as set forth in SEQ ID NO:361; and
a CDR L1 as set forth in SEQ ID NO:362, a CDR L2 as set forth in SEQ ID NO: 363, and a CDR L3 as set forth in SEQ ID NO:364.

23. The antibody of claim 1, wherein said antibody comprises:
a CDR H1 as set forth in SEQ ID NO:365, a CDR H2 as set forth in SEQ ID NO: 366 and a CDR H3 as set forth in SEQ ID NO:367; and
a CDR L1 as set forth in SEQ ID NO:368, a CDR L2 as set forth in SEQ ID NO: 369, and a CDR L3 as set forth in SEQ ID NO:370.

24. The antibody of claim 1, wherein said antibody comprises:
a CDR H1 as set forth in SEQ ID NO:371, a CDR H2 as set forth in SEQ ID NO: 372 and a CDR H3 as set forth in SEQ ID NO:373; and
a CDR L1 as set forth in SEQ ID NO:374, a CDR L2 as set forth in SEQ ID NO: 375, and a CDR L3 as set forth in SEQ ID NO:376.

25. The antibody of claim 1, wherein said antibody comprises:
a CDR H1 as set forth in SEQ ID NO:377, a CDR H2 as set forth in SEQ ID NO: 378 and a CDR H3 as set forth in SEQ ID NO:379; and
a CDR L1 as set forth in SEQ ID NO:380, a CDR L2 as set forth in SEQ ID NO: 381, and a CDR L3 as set forth in SEQ ID NO:382.

26. The antibody of claim 1, wherein said antibody comprises:
a CDR H1 as set forth in SEQ ID NO:383, a CDR H2 as set forth in SEQ ID NO: 384 and a CDR H3 as set forth in SEQ ID NO:385; and
a CDR L1 as set forth in SEQ ID NO:386, a CDR L2 as set forth in SEQ ID NO: 387, and a CDR L3 as set forth in SEQ ID NO:388.

27. The antibody of claim 1, wherein said antibody comprises:
a CDR H1 as set forth in SEQ ID NO:389, a CDR H2 as set forth in SEQ ID NO: 390 and a CDR H3 as set forth in SEQ ID NO:391; and
a CDR L1 as set forth in SEQ ID NO:392, a CDR L2 as set forth in SEQ ID NO: 393, and a CDR L3 as set forth in SEQ ID NO:394.

28. The antibody of claim 1, wherein said antibody is a humanized antibody.

29. The antibody of claim 1, wherein said antibody is a Fab′ fragment.

30. The antibody of claim 1, wherein said antibody is a single chain antibody (scFv).

31. The antibody of claim 1, wherein said antibody is a chimeric antibody.

32. The antibody of claim 1, wherein said antibody is an IgG.

33. The antibody of claim 1, wherein said antibody is an IgG1 or an IgG2.

34. The antibody of claim 1, wherein said antibody is capable of binding a ROR2 protein . . . .

35. The antibody of claim 1, wherein said antibody is capable of binding the extracellular domain of said ROR2 protein . . . .

36. The antibody of claim 35, wherein said extracellular domain is a Kringle domain.

37. The antibody of claim 34, wherein said ROR2 protein is expressed on a cell.

38. The antibody of claim 37, wherein said cell is a cancer cell.

39. The antibody of claim 38, wherein said cancer cell is a breast cancer cell, an ovarian cancer cell, a pancreatic cancer cell, a cervical cancer cell, a gastric cancer cell, a renal cancer cell, a head and neck cancer cell, a bone cancer cell, a skin cancer cell or a prostate cancer cell.

40. The antibody of claim 34, wherein said antibody is capable of binding said ROR2 protein with an equilibrium dissociation constant (KD) from about 0.02 nM to about 6 nM.

41. The antibody of claim 1, wherein said antibody is attached to a therapeutic agent.

42. The antibody of claim 1, wherein said antibody is attached to a diagnostic agent.

43. A method of treating cancer in a subject in need thereof, said method comprising administering to a subject a therapeutically effective amount of an antibody of claim 1.

44. The method of claim 43, wherein said cancer is breast cancer, ovarian cancer, pancreatic cancer, cervical cancer, gastric cancer, renal cancer, head and neck cancer, bone cancer, skin cancer or prostate cancer.

45. A method of detecting a ROR2 expressing cell, said method comprising
(i) contacting a ROR2-expressing cell with an antibody of claim 1; and
(ii) detecting binding of said antibody to a ROR2 protein expressed by said cell.

46. The method of claim 45, wherein said antibody is attached to a detectable moiety.

47. The method of claim 45, wherein said ROR2-expressing cell is in a subject.

48. The method of claim 47, wherein said subject is a subject having or being at risk of having cancer.

49. The method of claim 48, wherein said cancer is breast cancer, ovarian cancer, pancreatic cancer, cervical cancer, gastric cancer, renal cancer, head and neck cancer, bone cancer, skin cancer or prostate cancer.

50. The method of claim 45, wherein said contacting occurs in vitro.

51. A method of delivering a therapeutic agent to a ROR2 expressing cell, said method comprising contacting a ROR2 expressing cell with an antibody of claim 1, wherein said antibody is attached to a therapeutic agent.

52. The method of claim 51, wherein said therapeutic agent is an anti-cancer agent.

53. The method of claim 51, wherein said ROR2-expressing cell is in a subject.

54. The method of claim 51, wherein said subject is a subject having or being at risk of having cancer.

55. The method of claim 54, wherein said cancer is breast cancer, ovarian cancer, pancreatic cancer, cervical cancer, gastric cancer, renal cancer, head and neck cancer, bone cancer, skin cancer or prostate cancer.

56. The method of claim 51, wherein said contacting occurs in vitro.

57. A method of inhibiting migration of a ROR2-expressing cell, said method comprising contacting a ROR2 expressing cell with an antibody of claim 1.

58. The method of claim 57, wherein said ROR2 expressing cell is in a subject having or being at risk of having cancer.

59. The method of claim 58, wherein said cancer is breast cancer, ovarian cancer, pancreatic cancer, cervical cancer, gastric cancer, renal cancer, head and neck cancer, bone cancer, skin cancer or prostate cancer.

60. The method of claim 57, wherein said contacting occurs in vitro.

61. The method of claim 57, wherein said ROR2 expressing cell is a cancer cell.

62. The method of claim 61, wherein said cancer cell is a breast cancer cell, an ovarian cancer cell, a pancreatic cancer cell, a cervical cancer cell, a gastric cancer cell, a renal cancer cell, a head and neck cancer cell, a bone cancer cell, a skin cancer cell or a prostate cancer cell.

63. An anti-ROR2 antibody, wherein said anti-ROR2 antibody binds the same epitope as an anti-ROR2 antibody of claim 1.

64. A chimeric antigen receptor comprising (i) an anti-ROR2 antibody of claim 1 and (ii) a transmembrane domain.

65. A method of treating cancer in a subject in need thereof said method comprising, administering a therapeutically effective amount of a chimeric antigen receptor of claim 64 to said subject.

66. The method of claim 65, wherein said cancer is breast cancer, ovarian cancer, pancreatic cancer, cervical cancer, gastric cancer, renal cancer, head and neck cancer, bone cancer, skin cancer or prostate cancer.
